(19) United States
(12) Patent Application Publication
(10) Pub. No.: US 2025/0123278 A1
(43) Pub. Date: Apr. 17, 2025

(54) METHODS OF ANALYZING A SAMPLE FOR CANCER-SPECIFIC IMMUNE CELLS

(71) Applicant: Immunoracle Inc., Natick, MA (US)

(72) Inventor: Yujiang Geno SHI, Boston, MA (US)

(21) Appl. No.: 18/704,409

(22) PCT Filed: Oct. 30, 2022
(86) PCT No.: PCT/US2022/078953

§ 371 Date: Apr. 24, 2024

Related U.S. Application Data
(60) Provisional application No. 63/273,768, filed on Oct. 29, 2021.

(51) Int. Cl. G01N 33/569; G01N 33/574

(52) U.S. Cl. CPC G01N 33/56972; G01N 33/57484; G01N 2333/70539

(57) ABSTRACT

The present application provides methods of analyzing a sample of an individual exhibiting no pathological symptoms of a cancer comprising contacting the sample with a bait composition that has a display moiety having a neoantigenic peptide under a condition sufficient for an immune cell to bind to the display moiety and isolating and analyzing the immune cell. Methods of detecting a cancer (such as a residual cancer) and methods of treatments are also provided.

### CROSS REFERENCE TO RELATED APPLICATIONS

This application claims priority benefit of U.S. Provisional Application 63/273,768 filed Oct. 29, 2021, the contents of which are incorporated herein by reference in their entirety.

### REFERENCE TO AN ELECTRONIC SEQUENCE LISTING

The contents of the electronic sequence listing (230062000140SEQLIST.xml; Size: 74,317 bytes; and Date of Creation: Oct. 25, 2022) is herein incorporated by reference in its entirety.

### FIELD

The application relates to methods of analyzing a sample for cancer-specific immune cells.

### BACKGROUND

The chances of survival for a patient with cancer are substantially improved if the disease is diagnosed and treated at an early clinical stage. This underpins the promise of early detection to improve prognosis. Unfortunately, effective screening tests for early detection do not exist for many cancers.

On the other hand, significant proportions of patients with successful treatment of cancer have minimal residual disease (MRD) which can progress to metastatic relapse. MRD is considered an essential prognostic factor in predicting the risk of relapse and the choice of post-remission therapy. However, MRD is often too minimal to be revealed by even the most sensitive medical imaging modalities, including the CT or MRI systems. Therefore, assessment of minimal residual disease needs to be a critical aspect of tumor surveillance and management in patients, especially in those with a high risk of disease relapse.

The disclosures of all publications, patents, patent applications and published patent applications referred to herein are hereby incorporated herein by reference in their entirety.

### BRIEF SUMMARY

The present application in one aspect provides a method of analyzing a sample of an individual exhibiting no pathological symptom of a cancer, the method comprising a) contacting the sample with a bait composition comprising a display moiety comprising a neoantigenic peptide under a condition sufficient for an immune cell to bind to the display moiety; b) isolating an immune cell associated with the display moiety; and c) analyzing the isolated immune cell. In some embodiments, the method further comprises culturing the isolated immune cell prior to the analyzing step. In some embodiments, the display moiety comprises two or more neoantigenic peptides. In some embodiments, the display moiety comprises four neoantigenic peptides.

In some embodiments according to any one of the methods described herein, the two or more neoantigenic peptides in the display moiety are the same.

In some embodiments according to any one of the methods described herein, the neoantigenic peptide has one or more of the following characteristics: a) having a binding affinity of about 1 nM to about 5000 nM (e.g., about 1 nM to about 50 nM, about 50 nM to about 500 nM, about 500 nM to about 5000 nM) to an MHC molecule; b) having a binding affinity of about 1 nM to about 5000 nM (e.g., about 1 nM to about 50 nM, about 50 nM to about 500 nM, about 500 nM to about 5000 nM) to a cognate TCR molecule (e.g., when bound to an MHC molecule); c) having a mutation relative to a wildtype peptide, optionally at the third amino acid position counting from the N-terminus; d) is hydrophobic; and e) has high content of aromatic residues.

In some embodiments according to any one of the methods described herein, the neoantigenic peptide has low immunogenicity.

In some embodiments according to any one of the methods described herein, the display moiety comprises an MHC molecule complexed with the neoantigenic peptide. In some embodiments, the MHC molecule is a MHC class I molecule. In some embodiments, the MHC class I molecule is selected from the group consisting of HLA-A, HLA-B, and HLA-C. In some embodiments, the peptide is about 8 to about 10 amino acids long. In some embodiments, the MHC is a MHC class II molecule. In some embodiments, the MHC class II molecule is selected from the group consisting of HLA-DQ and HLA-DR. In some embodiments, the neoantigenic peptide is about 10 to about 20 amino acids long.

In some embodiments according to any one of the methods described herein, the display moiety comprises a particle. In some embodiments, the particle is selected from the group consisting of: a surface, a nanoparticle, a bead, and a polymer. In some embodiments, the particle is a dextran particle. In some embodiments, the particle is a magnetic nanoparticle or polystyrene nanoparticle. In some embodiments, the particle is an agarose bead or a sepharose bead.

In some embodiments according to any one of the methods described herein, the neoantigenic peptide or MHC is directly attached to the particle.

In some embodiments according to any one of the methods described herein, the neoantigenic peptide or MHC is attached to the particle via a binding pair comprising a first binding component attached to the neoantigenic peptide and a second binding component bound to the particle.

In some embodiments according to any one of the methods described herein, the display moiety comprises a cell. In some embodiments, the cell comprises a polynucleotide encoding the neoantigenic peptide. In some embodiments, the polynucleotide encodes a plurality of neoantigenic peptides.

In some embodiments according to any one of the methods described herein, the display moiety further comprises a detectable label. In some embodiments, the detectable label is a fluorophore. In some embodiments, the isolating step comprises using fluorescence-activated cell sorting (FACS).

In some embodiments according to any one of the methods described herein, the isolating step comprises separating immune cells associated with the display moiety from the rest of the sample.

In some embodiments according to any one of the methods described herein, the isolated immune cell is selected from the group consisting of a cytotoxic T cell, a memory T cell, and a tumor infiltrating T cell.

In some embodiments according to any one of the methods described herein, the isolated immune cell is an isolated single immune cell.

In some embodiments according to any one of the methods described herein, the isolated immune cell is in a mixture of immune cells. In some embodiments, the mixture of immune cells is a mixture comprising T cells, memory T cells, macrophage cells, dendritic cells, or combinations thereof.

In some embodiments according to any one of the methods described herein, analyzing the isolated immune cell comprises detecting the isolated immune cell.

In some embodiments according to any one of the methods described herein, analyzing the isolated immune cell comprises quantifying the isolated immune cell in a sample or an enriched sample (including for example quantifying each of the different types of immune cells collectively or separately).

In some embodiments according to any one of the methods described herein, analyzing the isolated immune cell comprises sequencing one or more nucleic acids in the isolated immune cell. In some embodiments, analyzing the isolated immune cell further comprises analyzing the sequences of the one or more nucleic acids. In some embodiments, the one or more nucleic acids comprises a nucleic acid sequence is a TCR sequence. In some embodiments, analyzing the isolated immune cell comprises sequencing a plurality of nucleic acids in the isolated immune cell to obtain a profile (e.g., a gene expression profile, a gene mutation profile, or an epigenetic modification profile such as methylation profile) of desired nucleic acids. In some embodiments, analyzing the isolated immune cell comprises sequencing one or more nucleic acids in a plurality of isolated immune cells. In some embodiments, analyzing the isolated immune cell comprises sequencing a plurality of nucleic acids in a plurality of isolated immune cells to obtain a profile (e.g., a gene expression profile, a gene mutation profile, or an epigenetic modification profile such as methylation profile) of desired nucleic acids.

In some embodiments according to any one of the methods described herein, analyzing the sequences of the one or more nucleic acids comprises whole genome sequencing.

In some embodiments according to any one of the methods described herein, analyzing the sequences of the one or more nucleic acids comprises RNAseq sequencing.

In some embodiments according to any one of the methods described herein, analyzing the sequences of the one or more nucleic acids comprises a) obtaining an enriched sample from the isolated immune cell, wherein the enriched sample is enriched for the one or more nucleic acids; and b) sequencing the one or more nucleic acids in the enriched sample.

In some embodiments according to any one of the methods described herein, analyzing the isolated immune cell further comprises subjecting the isolated immune cell to mass spectrometry analysis, for example to obtain an epigenetic modification profile of the isolated immune cell.

In some embodiments according to any one of the methods described herein, analyzing the isolated immune cell further comprises identifying one or more epigenetic modifications in the isolated immune cell. In some embodiments, the one or more epigenetic modifications comprises DNA or RNA methylation, hydroxymethylation and/or histone modifications such as acetylation, methylation, and/or glycosylation.

In some embodiments according to any one of the methods described herein, the individual has not previously been diagnosed as having a cancer. In some embodiments, the individual is at risk of developing cancer. In some embodiments, the individual has been previously treated for cancer and exhibits no pathological symptom of a cancer after the treatment.

In some embodiments according to any one of the methods described herein, the individual is a human. In some embodiments, the individual is at least about any of 50, 55, 60, 65, 70, 75, or 80 years old.

In some embodiments according to any one of the methods described herein, the sample is selected from the group consisting of: blood, plasma, and a peripheral blood mononuclear cell (PMBC) sample.

In some embodiments according to any one of the methods described herein, the method further comprises generating a report comprising information about the cancer status in the individual. In some embodiments, the information about cancer status comprises: classification of cancer; type of cancer; nature of cancer; origin of cancer; stage of cancer; likelihood of cancer progression; likelihood of developing one or more cancer symptoms; molecular diagnosis; NGS pathology; and/or treatment options for the individual.

In some embodiments according to any one of the methods described herein, the bait composition comprises a plurality of different display moieties. In some embodiments, the each of the plurality of different display moieties in the bait composition comprises a different neoantigenic peptide. In some embodiments, the plurality of different display moieties in the bait composition comprises at least two different display moieties, each comprising a different MHC molecule. In some embodiments, the plurality of different display moieties in the bait composition comprises at least four different display moieties, each comprising a different MHC molecule. In some embodiments, the plurality of different display moieties in the bait composition comprises at least 100 different display moieties, each comprising a different MHC molecule.

In some embodiments according to any one of the methods described herein, each of the different display moieties comprising different MHC molecules comprises a different detectable label. In some embodiments, the detectable label is a fluorophore. In some embodiments, the isolating step comprises using fluorescence-activated cell sorting (FACS).

In some embodiments according to any one of the methods described herein, the isolating step comprises separating immune cells associated with each of the different display moieties comprising different MHC molecules into different populations.

In some embodiments according to any one of the methods described herein, the method comprises contacting each of a plurality of different display moieties with a sample from the individual separately and isolating the immune cell associated with each of the different display moiety.

In some embodiments, the method comprises: a) analyzing a pre-treatment sample from the individual prior to anti-cancer therapy and a post-treatment sample from the individual according to any one of the methods described herein; and b) identifying a difference in characteristics of the isolated immune cell from the pre-treatment sample and the isolated immune cell from the post-treatment sample.

The present application in another aspect provides a method of detecting cancer in an individual, comprising: analyzing a sample from the individual according to any one of the methods described herein, wherein a predetermined characteristic of the isolated immune cell is indicative of cancer in the individual. In some embodiments, the predetermined characteristic of the isolated immune cell comprises the presence of the isolated immune cell. In some embodiments, the predetermined characteristic of the isolated immune cell comprises a quantity of the isolated immune cell above a threshold level.

In some embodiments according to any one of the methods described herein, the predetermined characteristic of the isolated immune cell comprises a gene expression profile signature, a gene mutation profile signature, and/or an epigenetic modification signature. In some embodiments, the signature epigenetic modification comprises a DNA or RNA methylation, hydroxymethylation signature and/or a histone modification signature.

In some embodiments according to any one of the methods of detecting cancer in an individual described herein and wherein the individual has been previously treated with an anti-cancer therapy and exhibits no pathological symptom of cancer after treatment, the method comprises analyzing a post-treatment sample from the individual according to the methods described herein, wherein a predetermined characteristic of the isolated immune cell from the post-treatment sample is indicative of residual cancer in the individual. In some embodiments, the method comprises: a) analyzing a pre-treatment sample from the individual prior to anti-cancer therapy and a post-treatment sample from the individual according to the methods described herein, and b) comparing the characteristics of the isolated immune cells from the pre-treatment sample and isolated immune cells from the post-treatment sample; wherein a predetermined difference in characteristics of the isolated immune cell from the pre-treatment sample and the isolated immune cell from the post-treatment sample is indicative of residual cancer in the individual.

In some embodiments, a method of treating a cancer in an individual, comprising a) diagnosing the individual as having cancer according to the methods described herein; and b) subjecting the individual to an anti-cancer therapy. In some embodiments, the anti-cancer therapy is not an immunotherapy.

In some embodiments according to any one of the methods described herein, the cancer is a solid tumor.

In some embodiments according to any one of the methods described herein, the cancer is a carcinoma, a sarcoma, a myeloma, a leukemia, a lymphoma, a blastoma, a germ cell tumor, or any combination thereof.

In some embodiments according to any one of the methods described herein, the cancer is a squamous cell carcinoma or an adenocarcinoma.

### BRIEF DESCRIPTION OF DRAWINGS

FIG. 1 exemplifies newly predicated Kras neoantigens (SEQ ID NOs: 57-76) originated from the six most common Kras mutation (Kras G12V, Kras G12D, Kras G12R, Kras G12C, Kras G12I, Kras G12A).

FIG. 2 shows that four most common HLA-A, including HLA-A: 0201, HLA-A: 2402, HLA-A: 0301, HLA-A: 1101, and B2m were expressed and purified from E. coli. The target proteins were expressed in insoluble inclusion body (DPE).

FIG. 3 shows that the assembly of five common mutant Kras neoantigen library. Four types HLA (HLA-A: 0201, HLA-A: 2402, HLA-A: 0301, HLA-A: 1101) and four most common Kras mutation (Kras G12V, Kras G12D, Kras G12R, Kras G12C). The figure only presented part of the results of Kras G12V mutation.

FIG. 4 demonstrates the confirmation of luciferase intensity in constructed pancreatic tumor cell lines. D-luciferin was used as substrate. The signal was captured 15 min later after D-luciferin intraperitoneal injection.

FIG. 5 depicts tumor sizes 12 days post inoculation of 1×10⁵ or 1×10⁶ Pano2-Luc-GFP tumor cells (panel A) and tumor growth curve of Pano2-Luc-GFP tumor in C57BL/6J mice after 1×10⁶ tumor cells were subcutaneously challenged as measured by tumor volume (panel B).

FIG. 6 depicts construction of plasmid overexpression five common Kras mutation. The band size was about 750 bp. Two replicates were loaded, and the bands less than 50 bp were non-specific bands.

FIGS. 7A and 7B depicts the detection of Kras mutation neoantigen specific CD8⁺ T cells in peripheral blood of mice as early as Day 4 after intravenous tumor cell challenge of 4×10⁵ or 1×10⁶ cells.

FIG. 8 depicts the detection of Kras mutation neoantigen specific CD8⁺ T cells in peripheral blood of mice as early as Day 4 after intravenous tumor cell challenge of 1×10⁴ or 1×10⁵ cells.

FIGS. 9A and 9B depicts the detection of Kras mutation neoantigen specific CD8⁺ T cells in peripheral blood of mice as early as Day 4 after subcutaneous tumor challenge of 4×10⁵ cells. As shown, the Tetramer⁺CD8⁺ T cells population (ranged from 0.042% to 0.22, median: 0.0866%) was detectable in mice challenged with Pan02-n peptide, but no Tetramer⁺CD8⁺ T cells were detectable in mice challenge with Pan02-EV cells.

FIGS. 10A and 10B depicts the tumor growth curve of mice inoculated with 4×10⁵ cells Pan02-EV (without neoantigen expressing Kras mutation) and Pan02-n (with neoantigen expressing Kras mutation) tumor cells as assessed via bioluminescence (FIG. 9A) and the picture of mice at day 4 post tumor challenge evidencing no observation of tumor at the site of inoculation (red circle) (FIG. 9B).

FIGS. 11A-11C depict the presence or absence of Kras mutation neoantigen specific CD8⁺ T cells with Kras mutation specific Tetramer library in peripheral blood of pancreatic cancer patients (Patient #1-13).

FIG. 12 depicts of a summary of the presence or absence of CD8⁺ T cells in thirteen confirmed pancreatic patients with a presence or absence of a Kras mutation and a specific HLA-A phenotype. In this experiment, twenty G12D neoantigen peptides associated MHC tetramers were included in the bait composition but only four G12R neoantigen peptides associated tetramers were included.

### DETAILED DESCRIPTION

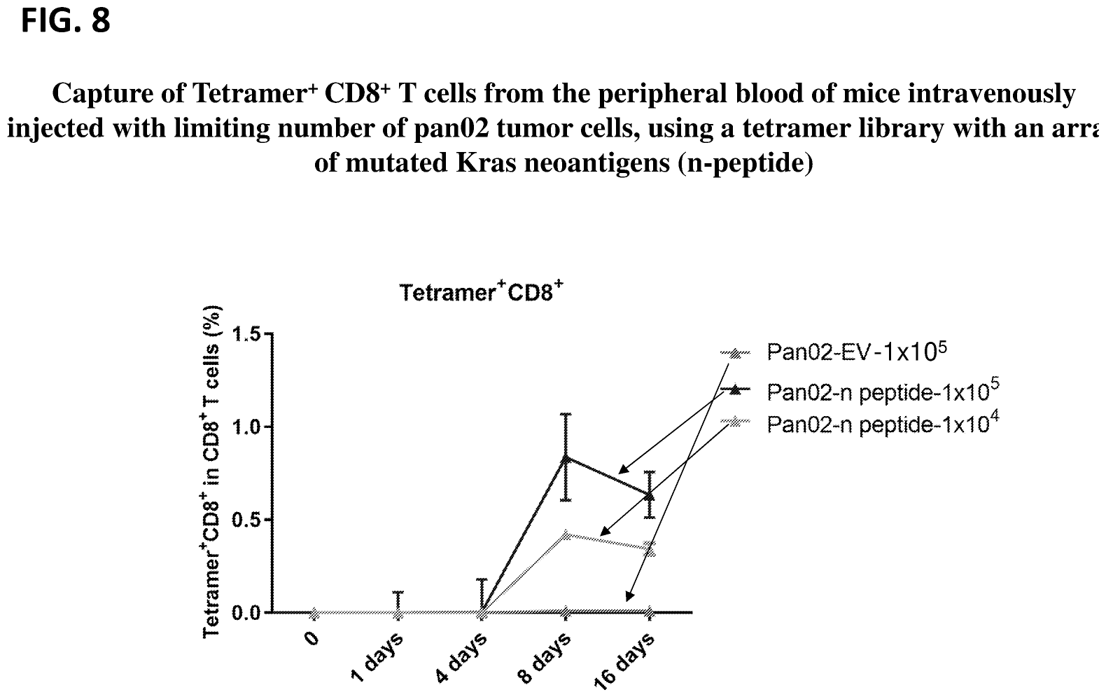

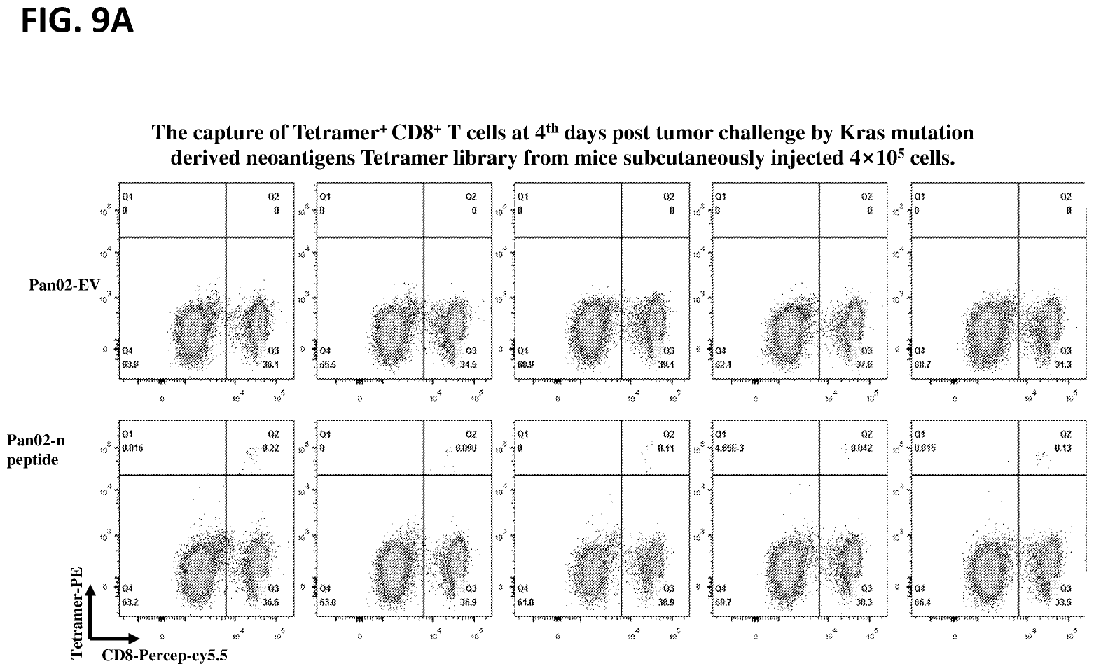

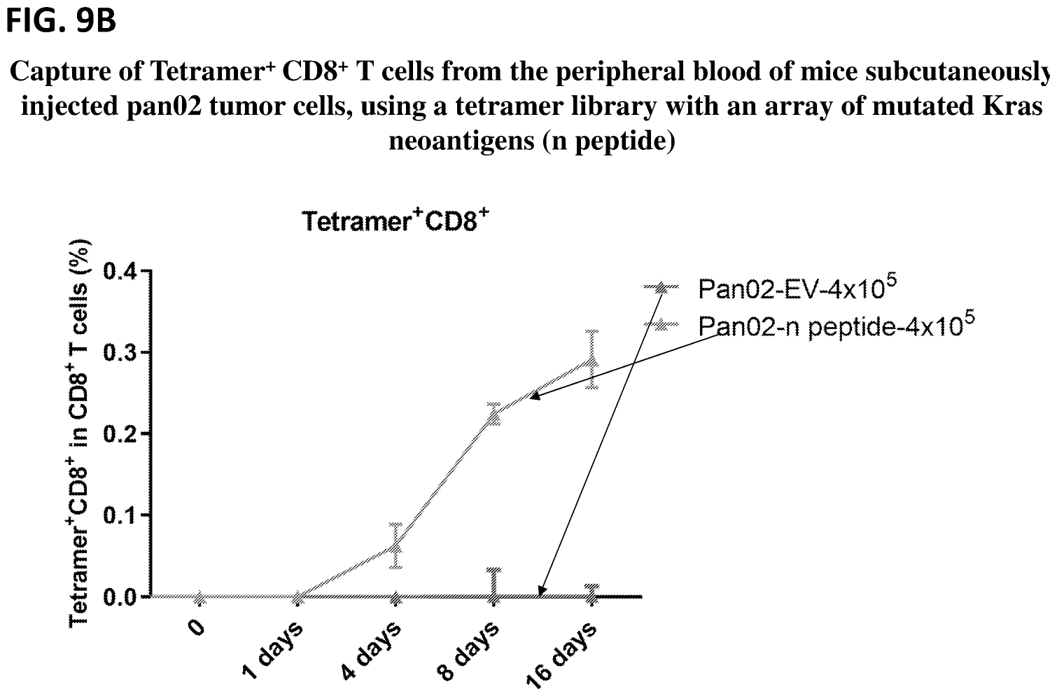

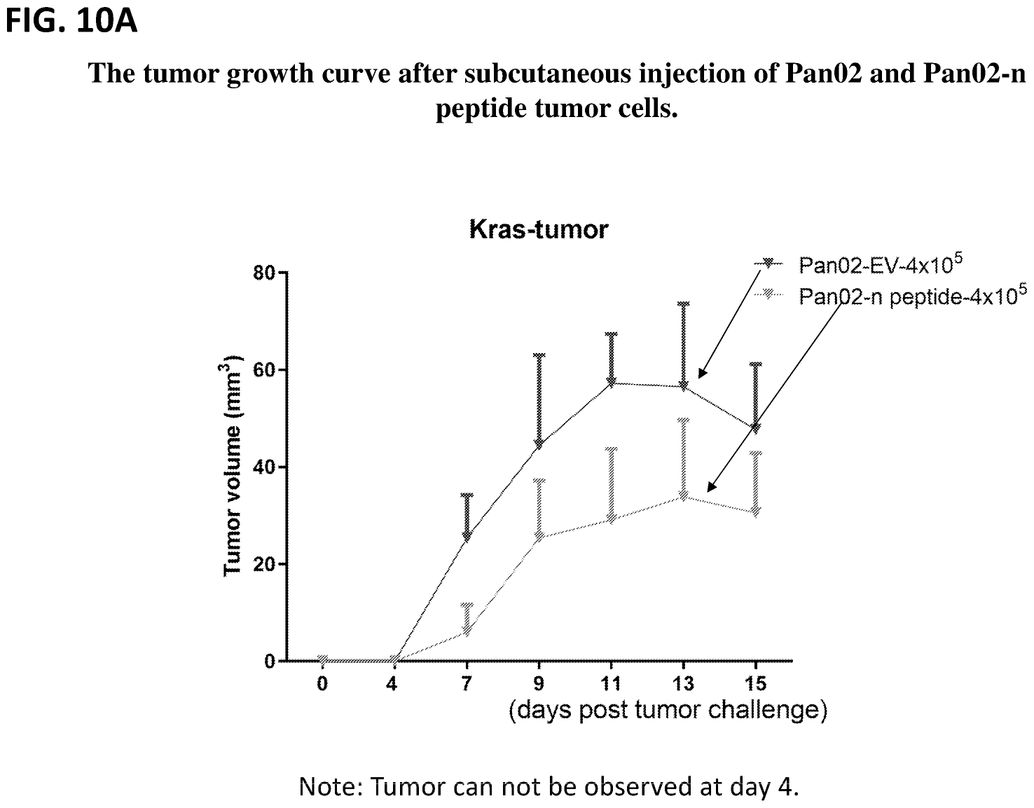

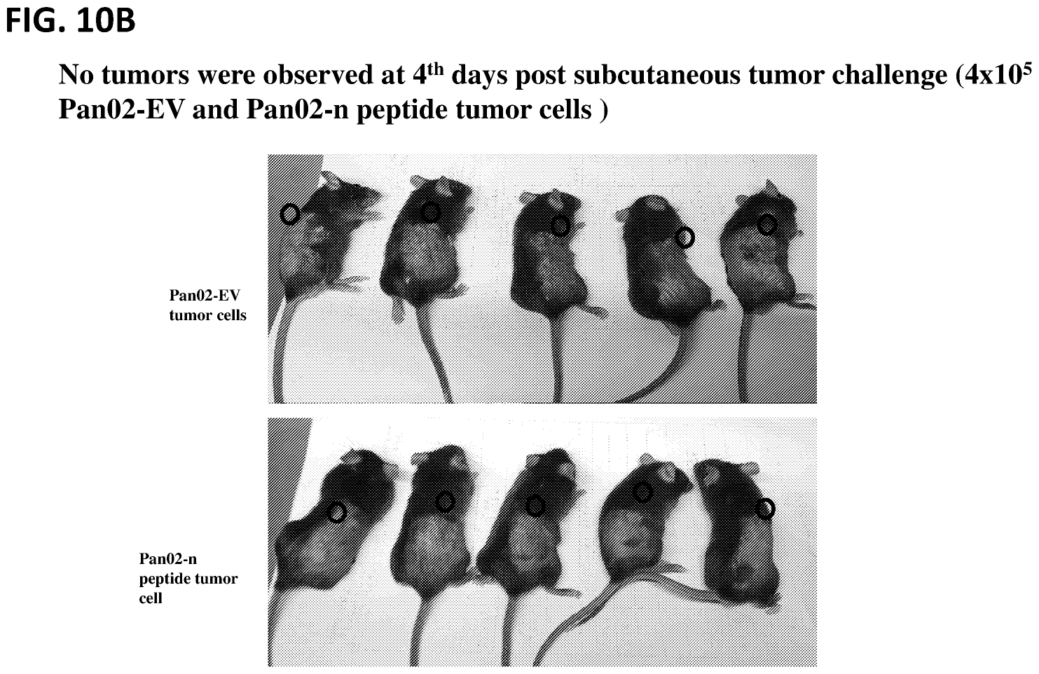

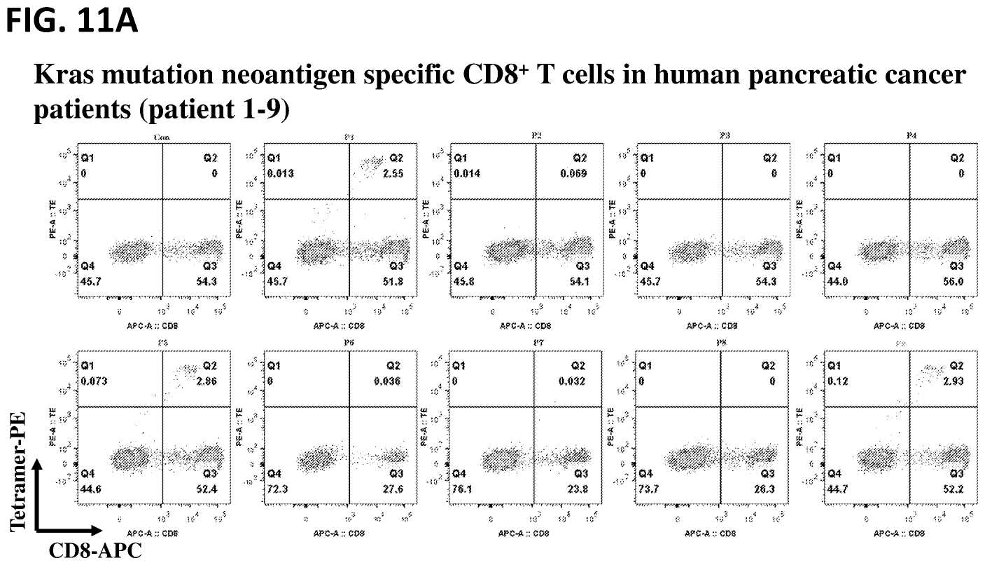

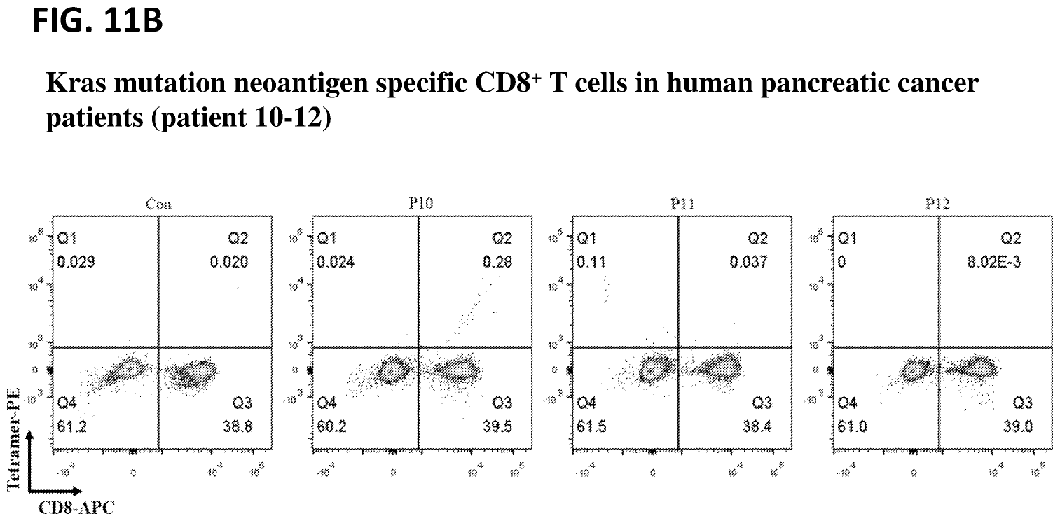

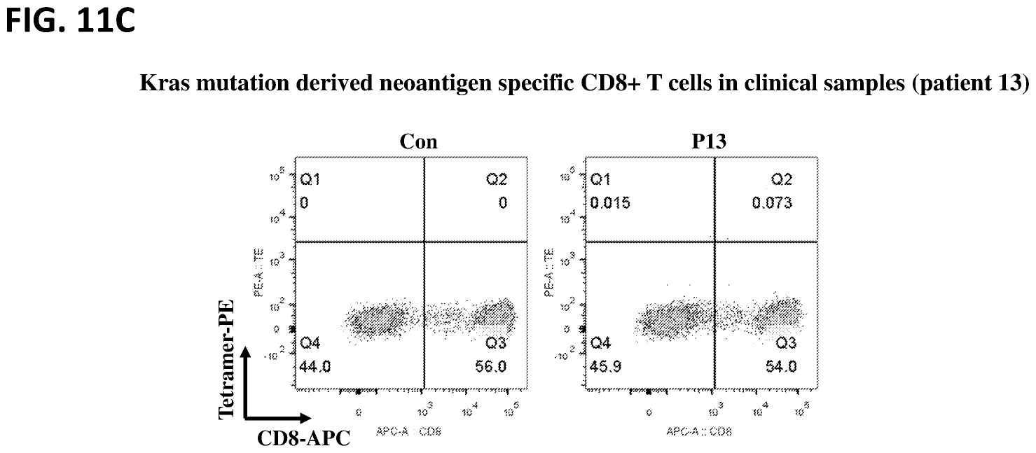

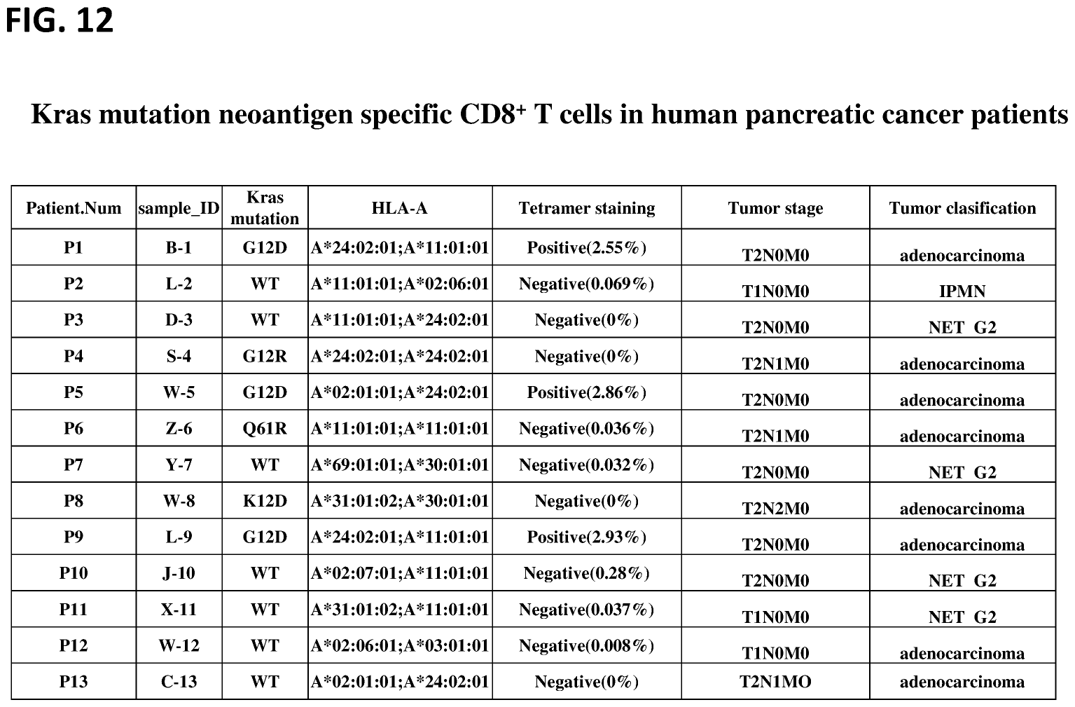

The present application provides methods of analyzing a sample of an individual exhibiting no pathological symptoms of a cancer. The application is based on inventors' unique insight that immune cells activated by tumor neoantigens can be detectable in an individual having cancer even before any pathological symptoms of cancer are exhibited, thus serving as useful markers for early cancer detection. As shown in Example section, exemplary Kras mutation associated neoantigen-specific T cells were successfully detected in mice intravenously or subcutaneously inoculated with tumor cells (of as low as 10⁴ cells, see, e.g., FIG. 8) expressing Kras mutation associated neoantigens and as early as day 4 after inoculation (see e.g., FIG. 9A-9B), which is prior to the any detection via bioluminescence (see e.g., FIG. 10A-10B), or in pancreatic cancer patients harboring Kras mutations (e.g., see FIGS. 11A-11C and 12). These data demonstrated that the provided methods can be successfully used for cancer detection including both early cancer detection as well as for monitoring cancer cells in individuals who may have residual minimal cancer cells.

The present application provides methods of isolating and analyzing immune cells in individuals exhibiting no pathological symptoms of a cancer, as well as methods of utilizing information so obtained for cancer screening in healthy individuals, for diagnosing or assisting in diagnosis of individuals suspected of having cancer, and for detecting minimal residual cancer (MRD) in individuals who has been previously treated with an anti-cancer therapy and exhibits no pathological symptom of cancer after treatment. More detailed analysis on the isolated immune cells can be further used for classifying cancer of the individual.

Thus, the present application in one aspect provides methods of analyzing a sample of an individual exhibiting no pathological symptoms of a cancer. In some embodiments, the method comprises a) contacting the sample with a bait composition comprising a display moiety comprising a neoantigenic peptide under a condition sufficient for an immune cell to bind to the display moiety; b) isolating an immune cell associated with the display moiety; and c) analyzing the isolated immune cell. In some embodiments, the individual has been previously treated for cancer and exhibits no pathological symptom of a cancer after the treatment. In some embodiments, the method comprises a) analyzing a pre-treatment sample from the individual prior to anti-cancer therapy and a post-treatment sample from the individual; and b) identifying a difference in characteristics of the isolated immune cell from the pre-treatment sample and the isolated immune cell from the post-treatment sample.

The present application in another aspect provides methods of detecting cancer in an individual exhibiting no pathological symptoms of a cancer, comprising: analyzing a sample from the individual, wherein a predetermined characteristic of the isolated immune cell is indicative of cancer in the individual.

The present application in another aspect provides methods of detecting residual cancer in an individual, wherein the individual has been previously treated with an anti-cancer therapy and exhibits no pathological symptom of cancer after treatment, the method comprising analyzing a post-treatment sample from the individual, wherein a predetermined characteristics of the isolated immune cell is indicative of residual cancer in the individual. A post-treatment sample refers to a sample harvested from an individual who has been subjected to a cancer treatment. In some embodiments, the post-treatment sample is harvested within about 1-3 weeks after treatment. In some embodiments, the post-treatment sample is harvested within about 1-3 months after treatment. In some embodiments, the post-treatment sample is harvested within about 6, 9, or 12 months after treatment.

The present application in another aspect provides method of treating a cancer in an individual, comprising a) diagnosing the individual as having cancer according to the diagnosis methods described herein; and b) subjecting the individual to an anti-cancer therapy.

### Definitions

In general, terms used in the claims and the specification are intended to be construed as having the plain meaning understood by a person of ordinary skill in the art. Certain terms are defined below to provide additional clarity. In case of conflict between the plain meaning and the provided definitions, the provided definitions are to be used.

As used herein the term “antigen” is a substance that induces an immune response.

As used herein the term “neoantigen” is an antigen that has at least one alteration that makes it distinct from the corresponding wild-type, parental antigen, e.g., via mutation in a tumor cell or post-translational modification specific to a tumor cell. A neoantigen can include a polypeptide sequence. A mutation that results in a neoantigen can include a frameshift or non-frameshift indel, missense or nonsense substitution, splice site alteration, genomic rearrangement or gene fusion, or any genomic or expression alteration giving rise to a neoORF. A mutations can also include a splice variant. Post-translational modifications specific to a tumor cell can include aberrant phosphorylation. Post-translational modifications specific to a tumor cell can also include a proteasome-generated spliced antigen. See Liepe et al., A large fraction of HLA class I ligands are proteasome-generated spliced peptides; Science. 2016 Oct. 21; 354(6310):354-358.

As used herein the term “tumor neoantigen” or “cancer neoantigen” is a neoantigen present in a subject's tumor cell or tissue but not in the subject's corresponding normal cell or tissue.

As used herein the term “missense mutation” is a mutation causing a substitution from one amino acid to another.

As used herein the term “nonsense mutation” is a mutation causing a substitution from an amino acid to a stop codon.

As used herein the term “frameshift mutation” is a mutation causing a change in the frame of the protein.

As used herein the term “indel” is an insertion or deletion of one or more nucleic acids.

As used herein, the term percent “identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. Alternatively, sequence similarity or dissimilarity can be established by the combined presence or absence of particular nucleotides, or, for translated sequences, amino acids at selected sequence positions (e.g., sequence motifs).

As used herein the term “epitope” is the specific portion of an antigen typically bound by an antibody or T-cell receptor.

As used herein the term “immunogenic” is the ability to elicit an immune response, e.g., via T-cells, B cells, or both.

As used herein the term “HLA binding affinity” “MHC binding affinity” means affinity of binding between a specific antigen and a specific MHC allele.

As used herein the term “bait composition” is a composition comprising a molecule (e.g., a neoantigen peptide) used to enrich a cell that specifically binds to the bait from a sample.

As used herein the term “variant” is a difference between a subject's nucleic acids and the reference human genome used as a control.

As used herein the term “allele” is a version of a gene or a version of a genetic sequence or a version of a protein.

As used herein the term “HLA type” is the complement of HLA gene alleles.

As used herein the term “exome” is a subset of the genome that codes for proteins. An exome can be the collective exons of a genome.

As used herein the term “proteome” is the set of all proteins expressed and/or translated by a cell, group of cells, or individual.

As used herein the term “dextramers” is a dextran-based peptide-MHC multimers used for antigen-specific immune-cell staining in flow cytometry.

As used herein the term “MHC multimers” is a peptide-MHC complex comprising multiple peptide-MHC monomer units.

As used herein the term “MHC tetramers” is a peptide-MHC complex comprising four peptide-MHC monomer units.

As used herein, “sample” refers to an aliquot of body fluid or a tissue obtained from a subject which contains an immune cell.

The term “mammal” encompasses both humans and non-humans and includes but is not limited to humans, non-human primates, canines, felines, murines, bovines, equines, and porcines.

As used herein, “treatment” or “treating” is an approach for obtaining beneficial or desired results, including clinical results. For purposes of this application, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, delaying or slowing the progression of the disease, ameliorating the disease state, providing a remission (partial or total) of the disease, decreasing the dose of one or more other medications required to treat the disease, delaying the progression of the disease, increasing or improving the quality of life, increasing weight gain, and/or prolonging survival. Also encompassed by “treatment” is a reduction of pathological consequence of cancer (such as, for example, tumor volume). The methods of the application contemplate any one or more of these aspects of treatment.

A “reference” as used herein, refers to any sample, standard, or level that is used for comparison purposes. A reference may be obtained from a healthy and/or non-diseased sample. In some examples, a reference may be obtained from an untreated sample. In some examples, a reference is obtained from a non-diseased or non-treated sample of an individual. In some examples, a reference is obtained from one or more healthy individuals who are not the individual or patient.

The terms “subject.” “individual.” and “patient” are used interchangeably herein to refer to a mammal, including, but not limited to, human, bovine, horse, feline, canine, rodent, or primate. In some embodiments, the individual is a human.

It is understood that embodiments of the application described herein include “consisting” and/or “consisting essentially of” embodiments.

Reference to “about” a value or parameter herein includes (and describes) variations that are directed to that value or parameter per se. For example, description referring to “about X” includes description of “X”.

As used herein, reference to “not” a value or parameter generally means and describes “other than” a value or parameter. For example, the method is not used to treat cancer of type X means the method is used to treat cancer of types other than X.

The term “about X-Y” used herein has the same meaning as “about X to about Y.”

It should be noted that, as used in the specification and the appended claims, the singular forms “a,” “an,” and “the” include plural referents unless the context clearly dictates otherwise.

Any terms not directly defined herein shall be understood to have the meanings commonly associated with them as understood within the art of the invention. Certain terms are discussed herein to provide additional guidance to the practitioner in describing the compositions, devices, methods and the like of aspects of the invention, and how to make or use them. It will be appreciated that the same thing may be said in more than one way. Consequently, alternative language and synonyms may be used for any one or more of the terms discussed herein. No significance is to be placed upon whether or not a term is elaborated or discussed herein. Some synonyms or substitutable methods, materials and the like are provided. Recital of one or a few synonyms or equivalents does not exclude use of other synonyms or equivalents, unless it is explicitly stated. Use of examples, including examples of terms, is for illustrative purposes only and does not limit the scope and meaning of the aspects of the invention herein.

### Methods of Analyzing a Sample of an Individual Exhibiting No Pathological Symptoms of a Cancer

In some embodiments, there is provided a method of analyzing a sample (e.g., blood, plasma, or a PBMC sample) of an individual exhibiting no pathological symptoms of a cancer. In some embodiments, the method comprises a) contacting the sample with a bait composition comprising a display moiety comprising a neoantigenic peptide under a condition sufficient for an immune cell (e.g., a T cell, a cytotoxic T cell, a helper T cell, a memory T cell, and/or a tumor infiltrating T cell) to bind to the display moiety; b) isolating an immune cell associated with the display moiety; and c) analyzing the isolated immune cell. In some embodiments, the method further comprises culturing the isolated immune cell prior to the analyzing step. In some embodiments, the display moiety comprises a particle (e.g., a particle selected from the group consisting of: a surface, a nanoparticle, a bead, and a polymer). In some embodiments, the display moiety further comprises a detectable label. In some embodiments, the detectable label is a fluorophore. In some embodiments, analyzing the sequences of the one or more nucleic acids comprises whole genome sequencing, RNAseq sequencing, and/or subjecting the isolated immune cell to mass spectrometry analysis.

In some embodiments, there is provided a method of analyzing a sample (e.g., blood, plasma, or a PBMC sample) of an individual exhibiting no pathological symptoms of a cancer, wherein the method comprises a) contacting the sample with a bait composition comprising a display moiety comprising a neoantigenic peptide under a condition sufficient for an immune cell (e.g., a T cell, a cytotoxic T cell, a helper T cell, a memory T cell, and/or a tumor infiltrating T cell) to bind to the display moiety; b) isolating an immune cell associated with the display moiety; and c) analyzing the isolated immune cell, wherein the display moiety comprises two or more (e.g., four) neoantigenic peptides. In some embodiments, the two or more neoantigenic peptides in the display moiety are the same. In some embodiments, the neoantigenic peptide has one or more of the following characteristics: a) having a binding affinity of about 1 nM to about 5000 nM (e.g., about 1 nM to about 50 nM, about 50 nM to about 500 nM, about 500 nM to about 5000 nM) to an MHC molecule; b) having a binding affinity of about 1 nM to about 5000 nM (e.g., about 1 nM to about 50 nM, about 50 nM to about 500 nM, about 500 nM to about 5000 nM) to a cognate TCR molecule; c) having a mutation relative to a wildtype peptide, optionally at the third amino acid position counting from the N-terminus; d) is hydrophobic; and e) has high content of aromatic residues. In some embodiments, the neoantigenic peptide has low immunogenicity. In some embodiments, the display moiety comprises an MHC molecule complexed with the neoantigenic peptide. In some embodiments, the MHC molecule is a MHC class I molecule and/or a MHC class II molecule. In some embodiments, the isolated immune cell is an isolated single immune cell. In some embodiments, the isolated immune cell is in a mixture of immune cells. In some embodiments, the mixture of immune cells is a mixture comprising T cells, memory T cells, macrophage cells, or dendritic cells, or combinations thereof. In some embodiments, analyzing the isolated immune cell comprises detecting and/or quantifying the isolated immune cell. In some embodiments, analyzing the isolated immune cell comprises sequencing one or more nucleic acids in the isolated immune cell, optionally further comprising analyzing the sequences of the one or more nucleic acids (e.g., a TCR related sequence). In some embodiments, analyzing the isolated immune cell further comprises identifying one or more epigenetic modifications (e.g., DNA or RNA methylation, hydroxymethylation, and/or histone modifications such as acetylation, methylation, glycosylation) in the isolated immune cell. In some embodiments, the method further comprises generating a report comprising information about the cancer status in the individual. In some embodiments, the information about cancer status comprises: classification of cancer; type of cancer; nature of cancer; origin of cancer; stage of cancer; likelihood of cancer progression; likelihood of developing one or more cancer symptoms; molecular diagnosis; NGS pathology; and/or treatment options for the individual. In some embodiments, the bait composition comprises a plurality of different display moieties. In some embodiments, each of the plurality of different display moieties in the bait composition comprises a different neoantigenic peptide (e.g., at least about two, four, 10, 25, 50, 75, or 100 different display moieties, each comprising a different MHC molecule). In some embodiments, each of the different display moieties comprising different MHC molecules comprises a different detectable label (e.g., a fluorophore). In some embodiments, the isolating step comprises using fluorescence-activated cell sorting (FACS), and/or separating immune cells associated with each of the different display moieties comprising different MHC molecules into different populations, optionally further comprises contacting each of a plurality of different display moieties with a sample from the individual separately and isolating the immune cell associated with each of the different display moiety. In some embodiments, the method further comprises culturing the isolated immune cell prior to the analyzing step. In some embodiments, the display moiety comprises a particle (e.g., a particle selected from the group consisting of: a surface, a nanoparticle, a bead, and a polymer). In some embodiments, the display moiety further comprises a detectable label. In some embodiments, the detectable label is a fluorophore. In some embodiments, analyzing the sequences of the one or more nucleic acids comprises whole genome sequencing, RNAseq sequencing, and/or subjecting the isolated immune cell to mass spectrometry analysis.

In some embodiments, there is provided a method of analyzing a sample (e.g., blood, plasma, or a PBMC sample) of an individual exhibiting no pathological symptoms of a cancer, wherein the method comprises a) contacting the sample with a bait composition comprising a display moiety comprising a neoantigenic peptide under a condition sufficient for an immune cell (e.g., a T cell, a cytotoxic T cell, a helper T cell, a memory T cell, and/or a tumor infiltrating T cell) to bind to the display moiety; b) isolating an immune cell associated with the display moiety; and c) analyzing the isolated immune cell, wherein the display moiety comprises an MHC molecule complexed with the neoantigenic peptide. In some embodiments, the MHC molecule is a MHC class I molecule. In some embodiments, the MHC class I molecule is selected from the group consisting of HLA-A, HLA-B, and HLA-C. In some embodiments, the peptide complexed with a MHC class I molecule is about 8 to about 10 amino acids long. In some embodiments, the MHC is a MHC class II molecule. In some embodiments, the MHC class II molecule is selected from the group consisting of HLA-DQ and HLA-DR. In some embodiments, the neoantigenic peptide complexed with the MHC class II molecule is about 10 to about 20 amino acids long. In some embodiments, the isolated immune cell is an isolated single immune cell. In some embodiments, the isolated immune cell is in a mixture of immune cells. In some embodiments, the mixture of immune cells is a mixture comprising T cells, memory T cells, macrophage cells, or dendritic cells, or combinations thereof. In some embodiments, analyzing the isolated immune cell comprises detecting and/or quantifying the isolated immune cell. In some embodiments, analyzing the isolated immune cell comprises sequencing one or more nucleic acids in the isolated immune cell, optionally further comprising analyzing the sequences of the one or more nucleic acids (e.g., a TCR related sequence). In some embodiments, analyzing the isolated immune cell further comprises identifying one or more epigenetic modifications (e.g., DNA or RNA methylation, hydroxymethylation, and/or histone modifications such as acetylation, methylation, glycosylation) in the isolated immune cell. In some embodiments, the method further comprises generating a report comprising information about the cancer status in the individual. In some embodiments, the information about cancer status comprises: classification of cancer; type of cancer; nature of cancer; origin of cancer; stage of cancer; likelihood of cancer progression; likelihood of developing one or more cancer symptoms; molecular diagnosis; NGS pathology; and/or treatment options for the individual. In some embodiments, the bait composition comprises a plurality of different display moieties. In some embodiments, each of the plurality of different display moieties in the bait composition comprises a different neoantigenic peptide (e.g., at least about two, four, 10, 25, 50, 75, or 100 different display moieties, each comprising a different MHC molecule). In some embodiments, each of the different display moieties comprising different MHC molecules comprises a different detectable label (e.g., a fluorophore). In some embodiments, the isolating step comprises using fluorescence-activated cell sorting (FACS), and/or separating immune cells associated with each of the different display moieties comprising different MHC molecules into different populations, optionally further comprises contacting each of a plurality of different display moieties with a sample from the individual separately and isolating the immune cell associated with each of the different display moiety. In some embodiments, the method further comprises culturing the isolated immune cell prior to the analyzing step. In some embodiments, the display moiety comprises a particle (e.g., a particle selected from the group consisting of: a surface, a nanoparticle, a bead, and a polymer). In some embodiments, the display moiety further comprises a detectable label. In some embodiments, the detectable label is a fluorophore. In some embodiments, analyzing the sequences of the one or more nucleic acids comprises whole genome sequencing, RNAseq sequencing, and/or subjecting the isolated immune cell to mass spectrometry analysis.

In some embodiments, there is provided a method of analyzing a sample (e.g., blood, plasma, or a PBMC sample) of an individual exhibiting no pathological symptoms of a cancer, wherein the method comprises a) contacting the sample with a bait composition comprising a display moiety comprising a neoantigenic peptide under a condition sufficient for an immune cell (e.g., a T cell, a cytotoxic T cell, a helper T cell, a memory T cell, and/or a tumor infiltrating T cell) to bind to the display moiety; b) isolating an immune cell associated with the display moiety; and c) analyzing the isolated immune cell, wherein the isolated immune cell comprises a mixture of immune cells. In some embodiments, the mixture of immune cells is a mixture comprising T cells, memory T cells, macrophage cells, and/or dendritic cells, or combinations thereof. In some embodiments, analyzing the isolated immune cell comprises detecting and/or quantifying the isolated immune cell. In some embodiments, analyzing the isolated immune cell comprises sequencing one or more nucleic acids in the isolated immune cell, optionally further comprising analyzing the sequences of the one or more nucleic acids (e.g., a TCR related sequence). In some embodiments, analyzing the isolated immune cell further comprises identifying one or more epigenetic modifications (e.g., DNA or RNA methylation, hydroxymethylation, and/or histone modifications such as acetylation, methylation, glycosylation) in the isolated immune cell. In some embodiments, the method further comprises generating a report comprising information about the cancer status in the individual. In some embodiments, the information about cancer status comprises: classification of cancer; type of cancer; nature of cancer; origin of cancer; stage of cancer; likelihood of cancer progression; likelihood of developing one or more cancer symptoms; molecular diagnosis; NGS pathology; and/or treatment options for the individual. In some embodiments, the bait composition comprises a plurality of different display moieties. In some embodiments, each of the plurality of different display moieties in the bait composition comprises a different neoantigenic peptide (e.g., at least about two, four, 10, 25, 50, 75, or 100 different display moieties, each comprising a different MHC molecule). In some embodiments, each of the different display moieties comprising different MHC molecules comprises a different detectable label (e.g., a fluorophore). In some embodiments, the isolating step comprises using fluorescence-activated cell sorting (FACS), and/or separating immune cells associated with each of the different display moieties comprising different MHC molecules into different populations, optionally further comprises contacting each of a plurality of different display moieties with a sample from the individual separately and isolating the immune cell associated with each of the different display moiety. In some embodiments, the method further comprises culturing the isolated immune cell prior to the analyzing step. In some embodiments, the display moiety comprises a particle (e.g., a particle selected from the group consisting of: a surface, a nanoparticle, a bead, and a polymer). In some embodiments, the display moiety further comprises a detectable label. In some embodiments, the detectable label is a fluorophore. In some embodiments, analyzing the sequences of the one or more nucleic acids comprises whole genome sequencing, RNAseq sequencing, and/or subjecting the isolated immune cell to mass spectrometry analysis.

In some embodiments, there is provided a method of analyzing a sample (e.g., blood, plasma, or a PBMC sample) of an individual exhibiting no pathological symptoms of a cancer, wherein the method comprises a) contacting the sample with a bait composition comprising a display moiety comprising a neoantigenic peptide under a condition sufficient for an immune cell (e.g., a T cell, a cytotoxic T cell, a helper T cell, a memory T cell, and/or a tumor infiltrating T cell) to bind to the display moiety; b) isolating an immune cell associated with the display moiety; and c) analyzing the isolated immune cell, wherein analyzing the isolated immune cell comprises detecting and/or quantifying the isolated immune cell. In some embodiments, analyzing the isolated immune cell comprises sequencing one or more nucleic acids in the isolated immune cell, optionally further comprising analyzing the sequences of the one or more nucleic acids (e.g., a TCR related sequence). In some embodiments, analyzing the isolated immune cell further comprises identifying one or more epigenetic modifications (e.g., DNA or RNA methylation, hydroxymethylation, and/or histone modifications such as acetylation, methylation, glycosylation) in the isolated immune cell. In some embodiments, the method further comprises generating a report comprising information about the cancer status in the individual. In some embodiments, the information about cancer status comprises: classification of cancer; type of cancer; nature of cancer; origin of cancer; stage of cancer; likelihood of cancer progression; likelihood of developing one or more cancer symptoms; molecular diagnosis; NGS pathology; and/or treatment options for the individual. In some embodiments, the bait composition comprises a plurality of different display moieties. In some embodiments, each of the plurality of different display moieties in the bait composition comprises a different neoantigenic peptide (e.g., at least about two, four, 10, 25, 50, 75, or 100 different display moieties, each comprising a different MHC molecule). In some embodiments, each of the different display moieties comprising different MHC molecules comprises a different detectable label (e.g., a fluorophore). In some embodiments, the isolating step comprises using fluorescence-activated cell sorting (FACS), and/or separating immune cells associated with each of the different display moieties comprising different MHC molecules into different populations, optionally further comprises contacting each of a plurality of different display moieties with a sample from the individual separately and isolating the immune cell associated with each of the different display moiety. In some embodiments, the method further comprises culturing the isolated immune cell prior to the analyzing step. In some embodiments, the display moiety comprises a particle (e.g., a particle selected from the group consisting of: a surface, a nanoparticle, a bead, and a polymer). In some embodiments, the display moiety further comprises a detectable label. In some embodiments, the detectable label is a fluorophore. In some embodiments, analyzing the sequences of the one or more nucleic acids comprises whole genome sequencing, RNAseq sequencing, and/or subjecting the isolated immune cell to mass spectrometry analysis.

In some embodiments, there is provided a method of analyzing a sample (e.g., blood, plasma, or a PBMC sample) of an individual exhibiting no pathological symptoms of a cancer, wherein the method comprises a) contacting the sample with a bait composition comprising a display moiety comprising a neoantigenic peptide under a condition sufficient for an immune cell (e.g., a T cell, a cytotoxic T cell, a helper T cell, a memory T cell, and/or a tumor infiltrating T cell) to bind to the display moiety; b) isolating an immune cell associated with the display moiety; and c) analyzing the isolated immune cell, wherein analyzing the isolated immune cell comprises sequencing one or more nucleic acids in the isolated immune cell, optionally wherein analyzing the isolated immune cell further comprises analyzing the sequences of the one or more nucleic acids (e.g., a TCR related sequence). In some embodiments, analyzing the sequences of the one or more nucleic acids comprises: a) obtaining an enriched sample from the isolated immune cell, wherein the enriched sample is enriched for the one or more nucleic acids; and b) sequencing the one or more nucleic acids in the enriched sample. In some embodiments, analyzing the isolated immune cell further comprises identifying one or more epigenetic modifications (e.g., DNA or RNA methylation, hydroxymethylation, and/or histone modifications such as acetylation, methylation, glycosylation) in the isolated immune cell. In some embodiments, the method further comprises generating a report comprising information about the cancer status in the individual. In some embodiments, the information about cancer status comprises: classification of cancer; type of cancer; nature of cancer; origin of cancer; stage of cancer; likelihood of cancer progression; likelihood of developing one or more cancer symptoms; molecular diagnosis; NGS pathology; and/or treatment options for the individual. In some embodiments, the bait composition comprises a plurality of different display moieties. In some embodiments, each of the plurality of different display moieties in the bait composition comprises a different neoantigenic peptide (e.g., at least about two, four, 10, 25, 50, 75, or 100 different display moieties, each comprising a different MHC molecule). In some embodiments, each of the different display moieties comprising different MHC molecules comprises a different detectable label (e.g., a fluorophore). In some embodiments, the isolating step comprises using fluorescence-activated cell sorting (FACS), and/or separating immune cells associated with each of the different display moieties comprising different MHC molecules into different populations, optionally further comprises contacting each of a plurality of different display moieties with a sample from the individual separately and isolating the immune cell associated with each of the different display moiety. In some embodiments, the method further comprises culturing the isolated immune cell prior to the analyzing step. In some embodiments, the display moiety comprises a particle (e.g., a particle selected from the group consisting of: a surface, a nanoparticle, a bead, and a polymer). In some embodiments, the display moiety further comprises a detectable label. In some embodiments, the detectable label is a fluorophore. In some embodiments, analyzing the sequences of the one or more nucleic acids comprises whole genome sequencing, RNAseq sequencing, and/or subjecting the isolated immune cell to mass spectrometry analysis.

In some embodiments, there is provided a method of analyzing a sample (e.g., blood, plasma, or a PBMC sample) of an individual exhibiting no pathological symptoms of a cancer, wherein the method comprises a) contacting the sample with a bait composition comprising a display moiety comprising a neoantigenic peptide under a condition sufficient for an immune cell (e.g., a T cell, a cytotoxic T cell, a helper T cell, a memory T cell, and/or a tumor infiltrating T cell) to bind to the display moiety; b) isolating an immune cell associated with the display moiety; and c) analyzing the isolated immune cell, wherein analyzing the isolated immune cell comprises identifying one or more epigenetic modifications (e.g., DNA or RNA methylation, hydroxymethylation, and/or histone modifications such as acetylation, methylation, glycosylation) in the isolated immune cell. In some embodiments, the method further comprises generating a report comprising information about the cancer status in the individual. In some embodiments, the information about cancer status comprises: classification of cancer; type of cancer; nature of cancer; origin of cancer; stage of cancer; likelihood of cancer progression; likelihood of developing one or more cancer symptoms; molecular diagnosis; NGS pathology; and/or treatment options for the individual. In some embodiments, the bait composition comprises a plurality of different display moieties. In some embodiments, each of the plurality of different display moieties in the bait composition comprises a different neoantigenic peptide (e.g., at least about two, four, 10, 25, 50, 75, or 100 different display moieties, each comprising a different MHC molecule). In some embodiments, each of the different display moieties comprising different MHC molecules comprises a different detectable label (e.g., a fluorophore). In some embodiments, the isolating step comprises using fluorescence-activated cell sorting (FACS), and/or separating immune cells associated with each of the different display moieties comprising different MHC molecules into different populations, optionally further comprises contacting each of a plurality of different display moieties with a sample from the individual separately and isolating the immune cell associated with each of the different display moiety. In some embodiments, the method further comprises culturing the isolated immune cell prior to the analyzing step. In some embodiments, the display moiety comprises a particle (e.g., a particle selected from the group consisting of: a surface, a nanoparticle, a bead, and a polymer). In some embodiments, the display moiety further comprises a detectable label. In some embodiments, the detectable label is a fluorophore. In some embodiments, analyzing the sequences of the one or more nucleic acids comprises whole genome sequencing, RNAseq sequencing, and/or subjecting the isolated immune cell to mass spectrometry analysis.

In some embodiments, there is provided a method of analyzing a sample (e.g., blood, plasma, or a PBMC sample) of an individual exhibiting no pathological symptoms of a cancer. In some embodiments, the method comprises a) contacting the sample with a bait composition comprising a display moiety comprising a neoantigenic peptide under a condition sufficient for an immune cell (e.g., a T cell, a cytotoxic T cell, a helper T cell, a memory T cell, and/or a tumor infiltrating T cell) to bind to the display moiety; b) isolating an immune cell associated with the display moiety; and c) analyzing the isolated immune cell, wherein the method further comprises generating a report comprising information about the cancer status in the individual. In some embodiments, the information about cancer status comprises: classification of cancer; type of cancer; nature of cancer; origin of cancer; stage of cancer; likelihood of cancer progression; likelihood of developing one or more cancer symptoms; molecular diagnosis; NGS pathology; and/or treatment options for the individual. In some embodiments, the bait composition comprises a plurality of different display moieties. In some embodiments, each of the plurality of different display moieties in the bait composition comprises a different neoantigenic peptide (e.g., at least about two, four, 10, 25, 50, 75, or 100 different display moieties, each comprising a different MHC molecule). In some embodiments, each of the different display moieties comprising different MHC molecules comprises a different detectable label (e.g., a fluorophore). In some embodiments, the isolating step comprises using fluorescence-activated cell sorting (FACS), and/or separating immune cells associated with each of the different display moieties comprising different MHC molecules into different populations, optionally further comprises contacting each of a plurality of different display moieties with a sample from the individual separately and isolating the immune cell associated with each of the different display moiety. In some embodiments, the method further comprises culturing the isolated immune cell prior to the analyzing step. In some embodiments, the display moiety comprises a particle (e.g., a particle selected from the group consisting of: a surface, a nanoparticle, a bead, and a polymer). In some embodiments, the display moiety further comprises a detectable label. In some embodiments, the detectable label is a fluorophore. In some embodiments, analyzing the sequences of the one or more nucleic acids comprises whole genome sequencing, RNAseq sequencing, and/or subjecting the isolated immune cell to mass spectrometry analysis.

In some embodiments, there is provided a method of analyzing a sample (e.g., blood, plasma, or a PBMC sample) of an individual exhibiting no pathological symptoms of a cancer. In some embodiments, the method comprises a) contacting the sample with a bait composition comprising a display moiety comprising a neoantigenic peptide under a condition sufficient for an immune cell (e.g., a T cell, a cytotoxic T cell, a helper T cell, a memory T cell, and/or a tumor infiltrating T cell) to bind to the display moiety; b) isolating an immune cell associated with the display moiety; and c) analyzing the isolated immune cell, wherein the bait composition comprises a plurality of different display moieties. In some embodiments, each of the plurality of different display moieties in the bait composition comprises a different neoantigenic peptide (e.g., at least about two, four, 10, 25, 50, 75, or 100 different display moieties, each comprising a different MHC molecule). In some embodiments, each of the different display moieties comprising different MHC molecules comprises a different detectable label (e.g., a fluorophore). In some embodiments, the isolating step comprises using fluorescence-activated cell sorting (FACS). In some embodiments, the isolating step comprises separating immune cells associated with each of the different display moieties comprising different MHC molecules into different populations. In some embodiments, the method further comprises contacting each of a plurality of different display moieties with a sample from the individual separately and isolating the immune cell associated with each of the different display moiety. In some embodiments, the method further comprises culturing the isolated immune cell prior to the analyzing step. In some embodiments, the display moiety comprises a particle (e.g., a particle selected from the group consisting of: a surface, a nanoparticle, a bead, and a polymer). In some embodiments, the display moiety further comprises a detectable label. In some embodiments, the detectable label is a fluorophore. In some embodiments, analyzing the sequences of the one or more nucleic acids comprises whole genome sequencing, RNAseq sequencing, and/or subjecting the isolated immune cell to mass spectrometry analysis.

In some embodiments, there is provided a method of analyzing a sample (e.g., blood, plasma, or a PBMC sample) of an individual exhibiting no pathological symptoms of a cancer, wherein the method comprises a) contacting the sample with a bait composition comprising a display moiety comprising a neoantigenic peptide under a condition sufficient for an immune cell to bind to the display moiety; b) isolating an immune cell associated with the display moiety; and c) analyzing the isolated immune cell, wherein the isolated immune cell is a T cell. In some embodiments, the T cell is a cytotoxic T cell. In some embodiments, the T cell is a helper T cell. In some embodiments, the T cell is a memory T cell. In some embodiments, the T cell is a tumor infiltrating T cell. In some embodiments, the display moiety comprises two or more (e.g., four) neoantigenic peptides. In some embodiments, the neoantigenic peptide has one or more of the following characteristics: a) having a binding affinity of about 1 nM to about 5000 nM (e.g., about 1 nM to about 50 nM, about 50 nM to about 500 nM, about 500 nM to about 5000 nM) to an MHC molecule; b) having a binding affinity of about 1 nM to about 5000 nM (e.g., about 1 nM to about 50 nM, about 50 nM to about 500 nM, about 500 nM to about 5000 nM) to a cognate TCR molecule; c) having a mutation relative to a wildtype peptide, optionally at the third amino acid position counting from the N-terminus; d) is hydrophobic; and e) has high content of aromatic residues. In some embodiments, the neoantigenic peptide has low immunogenicity. In some embodiments, the display moiety comprises an MHC molecule complexed with the neoantigenic peptide. In some embodiments, the MHC molecule is a MHC class I molecule and/or a MHC class II molecule. In some embodiments, analyzing the isolated immune cell comprises detecting and/or quantifying the isolated immune cell. In some embodiments, analyzing the isolated immune cell comprises sequencing one or more nucleic acids in the isolated immune cell, optionally further comprising analyzing the sequences of the one or more nucleic acids (e.g., a TCR related sequence). In some embodiments, analyzing the isolated immune cell further comprises identifying one or more epigenetic modifications (e.g., DNA or RNA methylation, hydroxymethylation, and/or histone modifications such as acetylation, methylation, glycosylation) in the isolated immune cell. In some embodiments, the method further comprises generating a report comprising information about the cancer status in the individual. In some embodiments, the information about cancer status comprises: classification of cancer; type of cancer; nature of cancer; origin of cancer; stage of cancer; likelihood of cancer progression; likelihood of developing one or more cancer symptoms; molecular diagnosis; NGS pathology; and/or treatment options for the individual. In some embodiments, the bait composition comprises a plurality of different display moieties. In some embodiments, each of the plurality of different display moieties in the bait composition comprises a different neoantigenic peptide (e.g., at least about two, four, 10, 25, 50, 75, or 100 different display moieties, each comprising a different MHC molecule). In some embodiments, each of the different display moieties comprising different MHC molecules comprises a different detectable label (e.g., a fluorophore). In some embodiments, the method further comprises culturing the isolated immune cell prior to the analyzing step. In some embodiments, the display moiety comprises a particle (e.g., a particle selected from the group consisting of: a surface, a nanoparticle, a bead, and a polymer). In some embodiments, the display moiety further comprises a detectable label (e.g., a fluorophore). In some embodiments, analyzing the sequences of the one or more nucleic acids comprises whole genome sequencing, RNAseq sequencing, and/or subjecting the isolated immune cell to mass spectrometry analysis.

In some embodiments, there is provided a method of analyzing a sample (e.g., blood, plasma, or a PBMC sample) of an individual exhibiting no pathological symptoms of a cancer, wherein the method comprises a) contacting the sample with a bait composition comprising a display moiety comprising a neoantigenic peptide under a condition sufficient for an immune cell to bind to the display moiety; b) isolating an immune cell associated with the display moiety; and c) analyzing the isolated immune cell, wherein the display moiety comprises an MHC molecule complexed with the neoantigenic peptide, wherein the MHC molecule is HLA-A * 24:02, HLA-A*11:01, HLA-A*02:01, or HLA-A*03:01. In some embodiments, the display moiety comprises two or more (such as two, three and four) different kinds of MHC class I molecules selected from the group consisting of HLA-A * 24:02, HLA-A*11:01, HLA-A*02:01, and HLA-A*03:01. In some embodiments, the display moiety further comprises a MHC class II molecule. In some embodiments, the display moiety does not comprise a MHC class II molecule. In some embodiments, the isolated immune cell is an isolated single immune cell. In some embodiments, the isolated immune cell is in a mixture of immune cells. In some embodiments, the mixture of immune cells is a mixture comprising T cells, memory T cells, macrophage cells, or dendritic cells, or combinations thereof. In some embodiments, the display moiety comprises two or more (e.g., four) neoantigenic peptides. In some embodiments, the neoantigenic peptide has one or more of the following characteristics: a) having a binding affinity of about 1 nM to about 5000 nM (e.g., about 1 nM to about 50 nM, about 50 nM to about 500 nM, about 500 nM to about 5000 nM) to an MHC molecule; b) having a binding affinity of about 1 nM to about 5000 nM (e.g., about 1 nM to about 50 nM, about 50 nM to about 500 nM, about 500 nM to about 5000 nM) to a cognate TCR molecule; c) having a mutation relative to a wildtype peptide, optionally at the third amino acid position counting from the N-terminus; d) is hydrophobic; and c) has high content of aromatic residues. In some embodiments, the neoantigenic peptide has low immunogenicity. In some embodiments, analyzing the isolated immune cell comprises detecting and/or quantifying the isolated immune cell. In some embodiments, analyzing the isolated immune cell comprises sequencing one or more nucleic acids in the isolated immune cell, optionally further comprising analyzing the sequences of the one or more nucleic acids (e.g., a TCR related sequence). In some embodiments, analyzing the isolated immune cell further comprises identifying one or more epigenetic modifications (e.g., DNA or RNA methylation, hydroxymethylation, and/or histone modifications such as acetylation, methylation, glycosylation) in the isolated immune cell. In some embodiments, the method further comprises generating a report comprising information about the cancer status in the individual. In some embodiments, the information about cancer status comprises: classification of cancer; type of cancer; nature of cancer; origin of cancer; stage of cancer; likelihood of cancer progression; likelihood of developing one or more cancer symptoms; molecular diagnosis; NGS pathology; and/or treatment options for the individual. In some embodiments, the bait composition comprises a plurality of different display moieties. In some embodiments, each of the plurality of different display moieties in the bait composition comprises a different neoantigenic peptide (e.g., at least about two, four, 10, 25, 50, 75, or 100 different display moieties, each comprising a different MHC molecule). In some embodiments, each of the different display moieties comprising different MHC molecules comprises a different detectable label (e.g., a fluorophore). In some embodiments, the method further comprises culturing the isolated immune cell prior to the analyzing step. In some embodiments, the display moiety comprises a particle (e.g., a particle selected from the group consisting of: a surface, a nanoparticle, a bead, and a polymer). In some embodiments, the display moiety further comprises a detectable label (e.g., a fluorophore). In some embodiments, analyzing the sequences of the one or more nucleic acids comprises whole genome sequencing, RNAseq sequencing, and/or subjecting the isolated immune cell to mass spectrometry analysis.

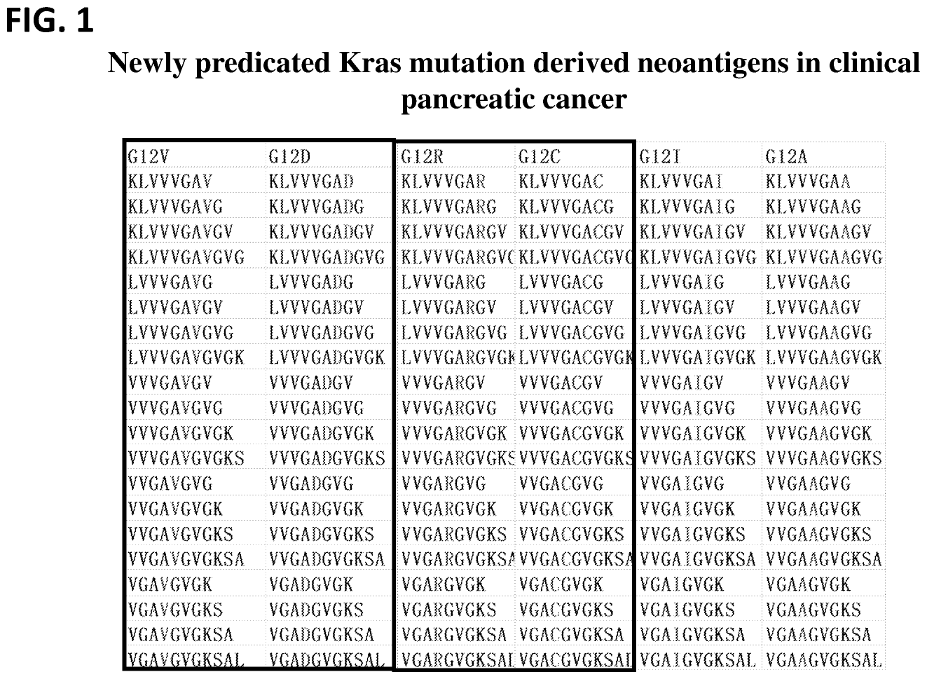

In some embodiments, there is provided a method of analyzing a sample (e.g., blood, plasma, or a PBMC sample) of an individual (e.g., an individual exhibiting no pathological symptoms of a cancer), wherein the method comprises a) contacting the sample with a bait composition comprising a display moiety comprising a plurality of neoantigenic peptides under a condition sufficient for an immune cell to bind to the display moiety; b) isolating an immune cell associated with the display moiety; and c) analyzing the isolated immune cell, wherein the display moiety comprises one or more MHC molecule complexed with the plurality of neoantigenic peptides, optionally wherein the MHC molecule is HLA-A * 24:02, HLA-A*11:01, HLA-A*02:01, or HLA-A*03:01. In some embodiments, at least one (e.g., each) of the plurality of neoantigen peptides comprises one or more known mutations associated with a cancer (e.g., Kras G12C, G12D, G12R, G12V, G12I, and/or G12A mutations). In some embodiments, the plurality of neoantigen peptides comprise at least 5, 10, 15, or 20 distinct neoantigen peptides each comprising a single known mutation associated with a cancer (e.g., Kras G12C, G12D, G12R, G12V, G12I, and/or G12A mutations). See FIG. 1 for exemplified design of a plurality of neoantigen peptides associated with a single known mutation. In some embodiments, the plurality of neoantigen peptides comprise at least 5, 10, 15, or 20 distinct neoantigen peptides associated with Kras G12C mutation (e.g., as shown in FIG. 1). In some embodiments, the plurality of neoantigen peptides comprise at least 5, 10, 15, or 20 distinct neoantigen peptides associated with Kras G12D mutation (e.g., as shown in FIG. 1). In some embodiments, the plurality of neoantigen peptides comprise at least 5, 10, 15, or 20 distinct neoantigen peptides associated with Kras G12R mutation (e.g., as shown in FIG. 1). In some embodiments, the plurality of neoantigen peptides comprise at least 5, 10, 15, or 20 distinct neoantigen peptides associated with Kras G12V mutation (e.g., as shown in FIG. 1). In some embodiments, the plurality of neoantigen peptides comprise at least 5, 10, 15, or 20 distinct neoantigen peptides associated with Kras G12I mutation (e.g., as shown in FIG. 1). In some embodiments, the plurality of neoantigen peptides comprise at least 5, 10, 15, or 20 distinct neoantigen peptides associated with Kras G12A mutation (e.g., as shown in FIG. 1). In some embodiments, the display moiety comprises two or more (such as two, three and four) different kinds of MHC class I molecules selected from the group consisting of HLA-A * 24:02, HLA-A*11:01, HLA-A*02:01, and HLA-A*03:01. In some embodiments, the display moiety further comprises a MHC class II molecule. In some embodiments, the display moiety does not comprise a MHC class II molecule. In some embodiments, the isolated immune cell is an isolated single immune cell. In some embodiments, the isolated immune cell is in a mixture of immune cells. In some embodiments, the mixture of immune cells is a mixture comprising T cells, memory T cells, macrophage cells, or dendritic cells, or combinations thereof. In some embodiments, the display moiety comprises two or more (e.g., four) neoantigenic peptides. In some embodiments, the neoantigenic peptide has one or more of the following characteristics: a) having a binding affinity of about 1 nM to about 5000 nM (e.g., about 1 nM to about 50 nM, about 50 nM to about 500 nM, about 500 nM to about 5000 nM) to an MHC molecule; b) having a binding affinity of about 1 nM to about 5000 nM (e.g., about 1 nM to about 50 nM, about 50 nM to about 500 nM, about 500 nM to about 5000 nM) to a cognate TCR molecule; c) having a mutation relative to a wildtype peptide, optionally at the third amino acid position counting from the N-terminus; d) is hydrophobic; and e) has high content of aromatic residues. In some embodiments, the neoantigenic peptide has low immunogenicity. In some embodiments, analyzing the isolated immune cell comprises detecting and/or quantifying the isolated immune cell. In some embodiments, analyzing the isolated immune cell comprises sequencing one or more nucleic acids in the isolated immune cell, optionally further comprising analyzing the sequences of the one or more nucleic acids (e.g., a TCR related sequence). In some embodiments, analyzing the isolated immune cell further comprises identifying one or more epigenetic modifications (e.g., DNA or RNA methylation, hydroxymethylation, and/or histone modifications such as acetylation, methylation, glycosylation) in the isolated immune cell. In some embodiments, the method further comprises generating a report comprising information about the cancer status in the individual. In some embodiments, the information about cancer status comprises: classification of cancer; type of cancer; nature of cancer; origin of cancer; stage of cancer; likelihood of cancer progression; likelihood of developing one or more cancer symptoms; molecular diagnosis; NGS pathology; and/or treatment options for the individual. In some embodiments, the bait composition comprises a plurality of different display moieties. In some embodiments, each of the plurality of different display moieties in the bait composition comprises a different neoantigenic peptide (e.g., at least about two, four, 10, 25, 50, 75, or 100 different display moieties, each comprising a different MHC molecule). In some embodiments, each of the different display moieties comprising different MHC molecules comprises a different detectable label (e.g., a fluorophore). In some embodiments, the method further comprises culturing the isolated immune cell prior to the analyzing step. In some embodiments, the display moiety comprises a particle (e.g., a particle selected from the group consisting of: a surface, a nanoparticle, a bead, and a polymer). In some embodiments, the display moiety further comprises a detectable label (e.g., a fluorophore). In some embodiments, analyzing the sequences of the one or more nucleic acids comprises whole genome sequencing, RNAseq sequencing, and/or subjecting the isolated immune cell to mass spectrometry analysis.

In some embodiments, there is provided a method of detecting (or identifying or assessing) the presence of cancer cells (e.g., minimal residual cancer cells, e.g., solid tumor cells) in an individual (e.g., an individual exhibiting no pathological symptoms of a cancer), wherein the method comprises a) contacting a sample (e.g., blood, plasma, or a PBMC sample) derived from the individual with a bait composition comprising a display moiety comprising a plurality of neoantigenic peptides under a condition sufficient for an immune cell to bind to the display moiety; b) isolating an immune cell associated with the display moiety; and c) analyzing the isolated immune cell, wherein the display moiety comprises one or more MHC molecule complexed with the plurality of neoantigenic peptides, optionally wherein the MHC molecule is HLA-A * 24:02, HLA-A*11:01, HLA-A*02:01, or HLA-A*03:01. In some embodiments, the individual has at least one cancer-related mutation. In some embodiments, at least one (e.g., each) of the plurality of neoantigen peptides comprise one or more known mutations associated with a cancer (e.g., Kras G12C, G12D, G12R, G12V, G12I, and/or G12A mutations). In some embodiments, the plurality of neoantigen peptides comprise at least 5, 10, 15, or 20 distinct neoantigen peptides each comprising a single known mutation associated with a cancer (e.g., Kras G12C, G12D, G12R, G12V, G12I, and/or G12A mutations). See FIG. 1 for exemplified design of a plurality of neoantigen peptides associated with a single known mutation. In some embodiments, the plurality of neoantigen peptides comprise at least 5, 10, 15, or 20 distinct neoantigen peptides associated with Kras G12C mutation (e.g., as shown in FIG. 1). In some embodiments, the plurality of neoantigen peptides comprise at least 5, 10, 15, or 20 distinct neoantigen peptides associated with Kras G12D mutation (e.g., as shown in FIG. 1). In some embodiments, the plurality of neoantigen peptides comprise at least 5, 10, 15, or 20 distinct neoantigen peptides associated with Kras G12R mutation (e.g., as shown in FIG. 1). In some embodiments, the plurality of neoantigen peptides comprise at least 5, 10, 15, or 20 distinct neoantigen peptides associated with Kras G12V mutation (e.g., as shown in FIG. 1). In some embodiments, the plurality of neoantigen peptides comprise at least 5, 10, 15, or 20 distinct neoantigen peptides associated with Kras G12I mutation (e.g., as shown in FIG. 1). In some embodiments, the plurality of neoantigen peptides comprise at least 5, 10, 15, or 20 distinct neoantigen peptides associated with Kras G12A mutation (e.g., as shown in FIG. 1). In some embodiments, the display moiety comprises two or more (such as two, three and four) different kinds of MHC class I molecules selected from the group consisting of HLA-A * 24:02, HLA-A*11:01, HLA-A*02:01, and HLA-A*03:01. In some embodiments, the display moiety further comprises a MHC class II molecule. In some embodiments, the display moiety does not comprise a MHC class II molecule. In some embodiments, the isolated immune cell is an isolated single immune cell. In some embodiments, the isolated immune cell is in a mixture of immune cells. In some embodiments, the mixture of immune cells is a mixture comprising T cells, memory T cells, macrophage cells, or dendritic cells, or combinations thereof. In some embodiments, the display moiety comprises two or more (e.g., four) neoantigenic peptides. In some embodiments, the neoantigenic peptide has one or more of the following characteristics: a) having a binding affinity of about 1 nM to about 5000 nM (e.g., about 1 nM to about 50 nM, about 50 nM to about 500 nM, about 500 nM to about 5000 nM) to an MHC molecule; b) having a binding affinity of about 1 nM to about 5000 nM (e.g., about 1 nM to about 50 nM, about 50 nM to about 500 nM, about 500 nM to about 5000 nM) to a cognate TCR molecule; c) having a mutation relative to a wildtype peptide, optionally at the third amino acid position counting from the N-terminus; d) is hydrophobic; and c) has high content of aromatic residues. In some embodiments, the neoantigenic peptide has low immunogenicity. In some embodiments, analyzing the isolated immune cell comprises detecting and/or quantifying the isolated immune cell. In some embodiments, analyzing the isolated immune cell comprises sequencing one or more nucleic acids in the isolated immune cell, optionally further comprising analyzing the sequences of the one or more nucleic acids (e.g., a TCR related sequence). In some embodiments, analyzing the isolated immune cell further comprises identifying one or more epigenetic modifications (e.g., DNA or RNA methylation, hydroxymethylation, and/or histone modifications such as acetylation, methylation, glycosylation) in the isolated immune cell. In some embodiments, the method further comprises generating a report comprising information about the cancer status in the individual. In some embodiments, the information about cancer status comprises: classification of cancer; type of cancer; nature of cancer; origin of cancer; stage of cancer; likelihood of cancer progression; likelihood of developing one or more cancer symptoms; molecular diagnosis; NGS pathology; and/or treatment options for the individual. In some embodiments, the bait composition comprises a plurality of different display moieties. In some embodiments, each of the plurality of different display moieties in the bait composition comprises a different neoantigenic peptide (e.g., at least about two, four, 10, 25, 50, 75, or 100 different display moieties, each comprising a different MHC molecule). In some embodiments, each of the different display moieties comprising different MHC molecules comprises a different detectable label (e.g., a fluorophore). In some embodiments, the method further comprises culturing the isolated immune cell prior to the analyzing step. In some embodiments, the display moiety comprises a particle (e.g., a particle selected from the group consisting of: a surface, a nanoparticle, a bead, and a polymer). In some embodiments, the display moiety further comprises a detectable label (e.g., a fluorophore). In some embodiments, analyzing the sequences of the one or more nucleic acids comprises whole genome sequencing, RNAseq sequencing, and/or subjecting the isolated immune cell to mass spectrometry analysis.

In some embodiments, the particle described herein is a dextran particle. In some embodiments, the particle is a magnetic nanoparticle or polystyrene nanoparticle. In some embodiments, the particle is an agarose bead or a sepharose bead. In some embodiments, the neoantigenic peptide or MHC is directly attached to the particle. In some embodiments, the neoantigenic peptide or MHC is attached to the particle via a binding pair comprising a first binding component attached to the neoantigenic peptide and a second binding component bound to the particle. In some embodiments, the display moiety comprises a cell. In some embodiments, the cell comprises a polynucleotide encoding the neoantigenic peptide. In some embodiments, the polynucleotide encodes a plurality of neoantigenic peptides.

In some embodiments, the individual is a human. In some embodiments, the individual is at least about 50 years old (e.g., at least 50, 60, 70, or 80 years old).

In some embodiments, the sample described herein (including this section and any other sections of the present application) can be any sample from the individual. In some embodiments, the sample described herein is a blood sample. In some embodiments, the sample described herein is a plasma sample. In some embodiments, the sample described herein comprises peripheral blood mononuclear cell (PMBC). In some embodiments, the sample described herein is a sample (e.g., a biopsy sample) obtained from a tissue or organ of the individual. In some embodiments, the sample is obtained from a lymph node of the individual.

### Methods of Detecting a Cancer in an Individual

In some embodiments, there is provided a method of detecting cancer in an individual (e.g., an individual not exhibiting a pathological symptom of cancer, e.g., an individual who has not been diagnosed as having a cancer, e.g., an individual being at risk of developing a cancer, e.g., an individual who has not been treated for cancer, e.g., an individual who has been treated with cancer and is suspected to have minimal residual cancer cells), comprising: analyzing a sample from the individual according to any of the methods described herein, wherein a predetermined characteristic of the isolated immune cell is indicative of cancer in the individual. In some embodiments, the predetermined characteristic of the isolated immune cell comprises the presence of the isolated immune cell. In some embodiments, the predetermined characteristic of the isolated immune cell comprises a quantity of the isolated immune cell above a threshold level. In some embodiments, the predetermined characteristic of the isolated immune cell comprises a gene expression profile signature, a gene mutation profile signature, and/or an epigenetic modification signature. In some embodiments, the signature epigenetic modification comprises a DNA methylation signature and a histone glycosylation signature.

In some embodiments, there is provided a method of detecting cancer in an individual (e.g., an individual who has not been diagnosed as having a cancer, e.g., an individual being at risk of developing a cancer, e.g., an individual who have been treated for cancer), comprising: a) contacting the sample with a bait composition comprising an display moiety comprising a cancer neoantigenic peptide under a condition sufficient for an immune cell (e.g., a T cell, a cytotoxic T cell, a helper T cell, a memory T cell, and/or a tumor infiltrating T cell) to bind to the display moiety; b) isolating an immune cell associated with the display moiety; and c) analyzing the isolated immune cell, wherein a predetermined characteristic of the isolated immune cell is indicative of cancer in the individual, and wherein the predetermined characteristic of the isolated immune cell comprises the presence of the isolated immune cell, optionally wherein the predetermined characteristic of the isolated immune cell comprises a quantity of the isolated immune cell above a threshold level. In some embodiments, the predetermined characteristic of the isolated immune cell further comprises a gene expression profile signature, a gene mutation profile signature, and/or an epigenetic modification signature (e.g., a DNA methylation signature and a histone glycosylation signature). In some embodiments, the display moiety comprises two or more (e.g., four) neoantigenic peptides. In some embodiments, the two or more neoantigenic peptides in the display moiety are the same. In some embodiments, the neoantigenic peptide has one or more of the following characteristics: a) having a binding affinity of about 1 nM to about 5000 nM (e.g., about 1 nM to about 50 nM, about 50 nM to about 500 nM, about 500 nM to about 5000 nM) to an MHC molecule; b) having a binding affinity of about 1 nM to about 5000 nM (e.g., about 1 nM to about 50 nM, about 50 nM to about 500 nM, about 500 nM to about 5000 nM) to a cognate TCR molecule; c) having a mutation relative to a wildtype peptide, optionally at the third amino acid position counting from the N-terminus; d) is hydrophobic; and e) has high content of aromatic residues. In some embodiments, the neoantigenic peptide has low immunogenicity. In some embodiments, the display moiety comprises an MHC molecule complexed with the neoantigenic peptide. In some embodiments, the MHC molecule is a MHC class I molecule and/or a MHC class II molecule. In some embodiments, the isolated immune cell is an isolated single immune cell. In some embodiments, the isolated immune cell is in a mixture of immune cells. In some embodiments, the mixture of immune cells is a mixture comprising T cells, memory T cells, macrophage cells, or dendritic cells, or combinations thereof. In some embodiments, analyzing the isolated immune cell comprises detecting and/or quantifying the isolated immune cell. In some embodiments, analyzing the isolated immune cell comprises sequencing one or more nucleic acids in the isolated immune cell, optionally further comprising analyzing the sequences of the one or more nucleic acids (e.g., a TCR related sequence). In some embodiments, analyzing the isolated immune cell further comprises identifying one or more epigenetic modifications (e.g., DNA or RNA methylation, hydroxymethylation, and/or histone modifications such as acetylation, methylation, glycosylation) in the isolated immune cell. In some embodiments, the method further comprises generating a report comprising information about the cancer status in the individual. In some embodiments, the information about cancer status comprises: classification of cancer; type of cancer; nature of cancer; origin of cancer; stage of cancer; likelihood of cancer progression; likelihood of developing one or more cancer symptoms; molecular diagnosis; NGS pathology; and/or treatment options for the individual. In some embodiments, the bait composition comprises a plurality of different display moieties. In some embodiments, each of the plurality of different display moieties in the bait composition comprises a different neoantigenic peptide (e.g., at least about two, four, 10, 25, 50, 75, or 100 different display moieties, each comprising a different MHC molecule). In some embodiments, each of the different display moieties comprising different MHC molecules comprises a different detectable label (e.g., a fluorophore). In some embodiments, the isolating step comprises using fluorescence-activated cell sorting (FACS), and/or separating immune cells associated with each of the different display moieties comprising different MHC molecules into different populations, optionally further comprises contacting each of a plurality of different display moieties with a sample from the individual separately and isolating the immune cell associated with each of the different display moiety. In some embodiments, the method further comprises culturing the isolated immune cell prior to the analyzing step. In some embodiments, the display moiety comprises a particle (e.g., a particle selected from the group consisting of: a surface, a nanoparticle, a bead, and a polymer). In some embodiments, the display moiety further comprises a detectable label. In some embodiments, the detectable label is a fluorophore. In some embodiments, analyzing the sequences of the one or more nucleic acids comprises whole genome sequencing, RNAseq sequencing, and/or subjecting the isolated immune cell to mass spectrometry analysis.

In some embodiments, there is provided a method of detecting cancer in an individual (e.g., an individual who has not been diagnosed as having a cancer, e.g., an individual being at risk of developing a cancer, e.g., an individual who have been treated for cancer), comprising: a) contacting the sample with a bait composition comprising an display moiety comprising a cancer neoantigenic peptide under a condition sufficient for an immune cell (e.g., a T cell, a cytotoxic T cell, a helper T cell, a memory T cell, and/or a tumor infiltrating T cell) to bind to the display moiety; b) isolating an immune cell associated with the display moiety; and c) analyzing the isolated immune cell, wherein a predetermined characteristic of the isolated immune cell is indicative of cancer in the individual, and wherein the predetermined characteristic of the isolated immune cell comprise a gene expression profile signature. In some embodiments, the predetermined characteristic of the isolated immune cell further comprises a gene mutation profile signature, and/or an epigenetic modification signature (e.g., a DNA methylation signature and a histone glycosylation signature). In some embodiments, the display moiety comprises two or more (e.g., four) neoantigenic peptides. In some embodiments, the two or more neoantigenic peptides in the display moiety are the same. In some embodiments, the neoantigenic peptide has one or more of the following characteristics: a) having a binding affinity of about 1 nM to about 5000 nM (e.g., about 1 nM to about 50 nM, about 50 nM to about 500 nM, about 500 nM to about 5000 nM) to an MHC molecule; b) having a binding affinity of about 1 nM to about 5000 nM (e.g., about 1 nM to about 50 nM, about 50 nM to about 500 nM, about 500 nM to about 5000 nM) to a cognate TCR molecule; c) having a mutation relative to a wildtype peptide, optionally at the third amino acid position counting from the N-terminus; d) is hydrophobic; and e) has high content of aromatic residues. In some embodiments, the neoantigenic peptide has low immunogenicity. In some embodiments, the display moiety comprises an MHC molecule complexed with the neoantigenic peptide. In some embodiments, the MHC molecule is a MHC class I molecule and/or a MHC class II molecule. In some embodiments, the isolated immune cell is an isolated single immune cell. In some embodiments, the isolated immune cell is in a mixture of immune cells. In some embodiments, the mixture of immune cells is a mixture comprising T cells, memory T cells, macrophage cells, or dendritic cells, or combinations thereof. In some embodiments, analyzing the isolated immune cell comprises detecting and/or quantifying the isolated immune cell. In some embodiments, analyzing the isolated immune cell comprises sequencing one or more nucleic acids in the isolated immune cell, optionally further comprising analyzing the sequences of the one or more nucleic acids (e.g., a TCR related sequence). In some embodiments, analyzing the isolated immune cell further comprises identifying one or more epigenetic modifications (e.g., DNA or RNA methylation, hydroxymethylation, and/or histone modifications such as acetylation, methylation, glycosylation) in the isolated immune cell. In some embodiments, the method further comprises generating a report comprising information about the cancer status in the individual. In some embodiments, the information about cancer status comprises: classification of cancer; type of cancer; nature of cancer; origin of cancer; stage of cancer; likelihood of cancer progression; likelihood of developing one or more cancer symptoms; molecular diagnosis; NGS pathology; and/or treatment options for the individual. In some embodiments, the bait composition comprises a plurality of different display moieties. In some embodiments, each of the plurality of different display moieties in the bait composition comprises a different neoantigenic peptide (e.g., at least about two, four, 10, 25, 50, 75, or 100 different display moieties, each comprising a different MHC molecule). In some embodiments, each of the different display moieties comprising different MHC molecules comprises a different detectable label (e.g., a fluorophore). In some embodiments, the isolating step comprises using fluorescence-activated cell sorting (FACS), and/or separating immune cells associated with each of the different display moieties comprising different MHC molecules into different populations, optionally further comprises contacting each of a plurality of different display moieties with a sample from the individual separately and isolating the immune cell associated with each of the different display moiety. In some embodiments, the method further comprises culturing the isolated immune cell prior to the analyzing step. In some embodiments, the display moiety comprises a particle (e.g., a particle selected from the group consisting of: a surface, a nanoparticle, a bead, and a polymer). In some embodiments, the display moiety further comprises a detectable label. In some embodiments, the detectable label is a fluorophore. In some embodiments, analyzing the sequences of the one or more nucleic acids comprises whole genome sequencing, RNAseq sequencing, and/or subjecting the isolated immune cell to mass spectrometry analysis.

In some embodiments, there is provided a method of detecting cancer in an individual (e.g., an individual who has not been diagnosed as having a cancer, e.g., an individual being at risk of developing a cancer, e.g., an individual who have been treated for cancer), comprising: a) contacting the sample with a bait composition comprising an display moiety comprising a cancer neoantigenic peptide under a condition sufficient for an immune cell (e.g., a T cell, a cytotoxic T cell, a helper T cell, a memory T cell, and/or a tumor infiltrating T cell) to bind to the display moiety; b) isolating an immune cell associated with the display moiety; and c) analyzing the isolated immune cell, wherein a predetermined characteristic of the isolated immune cell is indicative of cancer in the individual, and wherein the predetermined characteristic of the isolated immune cell comprises a gene mutation profile signature, and/or an epigenetic modification signature (e.g., a DNA methylation signature and a histone glycosylation signature). In some embodiments, the display moiety comprises two or more (e.g., four) neoantigenic peptides. In some embodiments, the two or more neoantigenic peptides in the display moiety are the same. In some embodiments, the neoantigenic peptide has one or more of the following characteristics: a) having a binding affinity of about 1 nM to about 5000 nM (e.g., about 1 nM to about 50 nM, about 50 nM to about 500 nM, about 500 nM to about 5000 nM) to an MHC molecule; b) having a binding affinity of about 1 nM to about 5000 nM (e.g., about 1 nM to about 50 nM, about 50 nM to about 500 nM, about 500 nM to about 5000 nM) to a cognate TCR molecule; c) having a mutation relative to a wildtype peptide, optionally at the third amino acid position counting from the N-terminus; d) is hydrophobic; and e) has high content of aromatic residues. In some embodiments, the neoantigenic peptide has low immunogenicity. In some embodiments, the display moiety comprises an MHC molecule complexed with the neoantigenic peptide. In some embodiments, the MHC molecule is a MHC class I molecule and/or a MHC class II molecule. In some embodiments, the isolated immune cell is an isolated single immune cell. In some embodiments, the isolated immune cell is in a mixture of immune cells. In some embodiments, the mixture of immune cells is a mixture comprising T cells, memory T cells, macrophage cells, or dendritic cells, or combinations thereof. In some embodiments, analyzing the isolated immune cell comprises detecting and/or quantifying the isolated immune cell. In some embodiments, analyzing the isolated immune cell comprises sequencing one or more nucleic acids in the isolated immune cell, optionally further comprising analyzing the sequences of the one or more nucleic acids (e.g., a TCR related sequence). In some embodiments, analyzing the isolated immune cell further comprises identifying one or more epigenetic modifications (e.g., DNA or RNA methylation, hydroxymethylation, and/or histone modifications such as acetylation, methylation, glycosylation) in the isolated immune cell. In some embodiments, the method further comprises generating a report comprising information about the cancer status in the individual. In some embodiments, the information about cancer status comprises: classification of cancer; type of cancer; nature of cancer; origin of cancer; stage of cancer; likelihood of cancer progression; likelihood of developing one or more cancer symptoms; molecular diagnosis; NGS pathology; and/or treatment options for the individual. In some embodiments, the bait composition comprises a plurality of different display moieties. In some embodiments, each of the plurality of different display moieties in the bait composition comprises a different neoantigenic peptide (e.g., at least about two, four, 10, 25, 50, 75, or 100 different display moieties, each comprising a different MHC molecule). In some embodiments, each of the different display moieties comprising different MHC molecules comprises a different detectable label (e.g., a fluorophore). In some embodiments, the isolating step comprises using fluorescence-activated cell sorting (FACS), and/or separating immune cells associated with each of the different display moieties comprising different MHC molecules into different populations, optionally further comprises contacting each of a plurality of different display moieties with a sample from the individual separately and isolating the immune cell associated with each of the different display moiety. In some embodiments, the method further comprises culturing the isolated immune cell prior to the analyzing step. In some embodiments, the display moiety comprises a particle (e.g., a particle selected from the group consisting of: a surface, a nanoparticle, a bead, and a polymer). In some embodiments, the display moiety further comprises a detectable label. In some embodiments, the detectable label is a fluorophore. In some embodiments, analyzing the sequences of the one or more nucleic acids comprises whole genome sequencing, RNAseq sequencing, and/or subjecting the isolated immune cell to mass spectrometry analysis.

In some embodiments, there is provided a method of detecting residual cancer in an individual, wherein the individual has been previously treated with an anti-cancer therapy and exhibits no pathological symptom of cancer after treatment, the method comprising analyzing a post-treatment sample from the individual. In some embodiments, the method further comprises a) analyzing a pre-treatment sample from the individual prior to anti-cancer therapy and a post-treatment sample from the individual according to any of the methods described herein, and b) comparing the characteristics of the isolated immune cells from the pre-treatment sample and isolated immune cells from the post-treatment sample. In some embodiments, a predetermined difference in characteristics of the isolated immune cell from the pre-treatment sample and the isolated immune cell from the post-treatment sample is indicative of residual cancer in the individual. In some embodiments, the predetermined characteristic of the isolated immune cell comprises the presence of the isolated immune cell, optionally further comprising a quantity of the isolated immune cell above a threshold level. In some embodiments, the predetermined characteristic of the isolated immune cell comprises a gene expression profile signature, a gene mutation profile signature, and/or an epigenetic modification signature (e.g., a DNA methylation signature and/or a histone glycosylation signature). In some embodiments, the method comprises: a) contacting the pre-treatment and/or post-treatment sample with a bait composition comprising an display moiety comprising a cancer neoantigenic peptide under a condition sufficient for an immune cell (e.g., a T cell, a cytotoxic T cell, a helper T cell, a memory T cell, and/or a tumor infiltrating T cell) to bind to the display moiety; b) isolating an immune cell associated with the display moiety; and c) analyzing the isolated immune cell. In some embodiments, the display moiety comprises two or more (e.g., four) neoantigenic peptides. In some embodiments, the two or more neoantigenic peptides in the display moiety are the same. In some embodiments, the neoantigenic peptide has one or more of the following characteristics: a) having a binding affinity of about 1 nM to about 5000 nM (e.g., about 1 nM to about 50 nM, about 50 nM to about 500 nM, about 500 nM to about 5000 nM) to an MHC molecule; b) having a binding affinity of about 1 nM to about 5000 nM (e.g., about 1 nM to about 50 nM, about 50 nM to about 500 nM, about 500 nM to about 5000 nM) to a cognate TCR molecule; c) having a mutation relative to a wildtype peptide, optionally at the third amino acid position counting from the N-terminus; d) is hydrophobic; and c) has high content of aromatic residues. In some embodiments, the neoantigenic peptide has low immunogenicity. In some embodiments, the display moiety comprises an MHC molecule complexed with the neoantigenic peptide. In some embodiments, the MHC molecule is a MHC class I molecule and/or a MHC class II molecule. In some embodiments, the isolated immune cell is an isolated single immune cell. In some embodiments, the isolated immune cell is in a mixture of immune cells. In some embodiments, the mixture of immune cells is a mixture comprising T cells, memory T cells, macrophage cells, or dendritic cells, or combinations thereof. In some embodiments, analyzing the isolated immune cell comprises detecting and/or quantifying the isolated immune cell. In some embodiments, analyzing the isolated immune cell comprises sequencing one or more nucleic acids in the isolated immune cell, optionally further comprising analyzing the sequences of the one or more nucleic acids (e.g., a TCR related sequence). In some embodiments, analyzing the isolated immune cell further comprises identifying one or more epigenetic modifications (e.g., DNA or RNA methylation, hydroxymethylation, and/or histone modifications such as acetylation, methylation, glycosylation) in the isolated immune cell. In some embodiments, the method further comprises generating a report comprising information about the cancer status in the individual. In some embodiments, the information about cancer status comprises: classification of cancer; type of cancer; nature of cancer; origin of cancer; stage of cancer; likelihood of cancer progression; likelihood of developing one or more cancer symptoms; molecular diagnosis; NGS pathology; and/or treatment options for the individual. In some embodiments, the bait composition comprises a plurality of different display moieties. In some embodiments, each of the plurality of different display moieties in the bait composition comprises a different neoantigenic peptide (e.g., at least about two, four, 10, 25, 50, 75, or 100 different display moieties, each comprising a different MHC molecule). In some embodiments, each of the different display moieties comprising different MHC molecules comprises a different detectable label (e.g., a fluorophore). In some embodiments, the isolating step comprises using fluorescence-activated cell sorting (FACS), and/or separating immune cells associated with each of the different display moieties comprising different MHC molecules into different populations, optionally further comprises contacting each of a plurality of different display moieties with a sample from the individual separately and isolating the immune cell associated with each of the different display moiety. In some embodiments, the method further comprises culturing the isolated immune cell prior to the analyzing step. In some embodiments, the display moiety comprises a particle (e.g., a particle selected from the group consisting of: a surface, a nanoparticle, a bead, and a polymer). In some embodiments, the display moiety further comprises a detectable label. In some embodiments, the detectable label is a fluorophore. In some embodiments, analyzing the sequences of the one or more nucleic acids comprises whole genome sequencing, RNAseq sequencing, and/or subjecting the isolated immune cell to mass spectrometry analysis.

### Methods of Treatment

In some embodiments, there is provided a method of treating a cancer in an individual (e.g., an individual who has not been diagnosed as having a cancer, e.g., an individual being at risk of developing a cancer, e.g., an individual who have been treated for cancer), comprising a) diagnosing the individual as having cancer according to the methods described herein; and b) subjecting the individual to an anti-cancer therapy. In some embodiments, diagnosing the individual comprises a) obtaining a sample from the individual, b) contacting the sample with a bait composition comprising an display moiety comprising a cancer neoantigenic peptide under a condition sufficient for an immune cell (e.g., a T cell, a cytotoxic T cell, a helper T cell, a memory T cell, and/or a tumor infiltrating T cell) to bind to the display moiety; c) isolating an immune cell associated with the display moiety; and d) analyzing the isolated immune cell, wherein a predetermined characteristic of the isolated immune cell is indicative of cancer in the individual. In some embodiments, the predetermined characteristic of the isolated immune cell comprises the presence of the isolated immune cell. In some embodiments, the predetermined characteristic of the isolated immune cell comprises a quantity of the isolated immune cell above a threshold level. In some embodiments, the predetermined characteristic of the isolated immune cell comprises a gene expression profile signature, a gene mutation profile signature, and/or an epigenetic modification signature (e.g., a DNA methylation signature and/or a histone glycosylation signature). In some embodiments, the display moiety comprises two or more (e.g., four) neoantigenic peptides. In some embodiments, the two or more neoantigenic peptides in the display moiety are the same. In some embodiments, the neoantigenic peptide has one or more of the following characteristics: a) having a binding affinity of about 1 nM to about 5000 nM (e.g., about 1 nM to about 50 nM, about 50 nM to about 500 nM, about 500 nM to about 5000 nM) to an MHC molecule; b) having a binding affinity of about 1 nM to about 5000 nM (e.g., about 1 nM to about 50 nM, about 50 nM to about 500 nM, about 500 nM to about 5000 nM) to a cognate TCR molecule; c) having a mutation relative to a wildtype peptide, optionally at the third amino acid position counting from the N-terminus; d) is hydrophobic; and c) has high content of aromatic residues. In some embodiments, the neoantigenic peptide has low immunogenicity. In some embodiments, the display moiety comprises an MHC molecule complexed with the neoantigenic peptide. In some embodiments, the MHC molecule is a MHC class I molecule and/or a MHC class II molecule. In some embodiments, the isolated immune cell is an isolated single immune cell. In some embodiments, the isolated immune cell is in a mixture of immune cells. In some embodiments, the mixture of immune cells is a mixture comprising T cells, memory T cells, macrophage cells, or dendritic cells, or combinations thereof. In some embodiments, analyzing the isolated immune cell comprises detecting and/or quantifying the isolated immune cell. In some embodiments, analyzing the isolated immune cell comprises sequencing one or more nucleic acids in the isolated immune cell, optionally further comprising analyzing the sequences of the one or more nucleic acids (e.g., a TCR related sequence). In some embodiments, analyzing the isolated immune cell further comprises identifying one or more epigenetic modifications (e.g., DNA or RNA methylation, hydroxymethylation, and/or histone modifications such as acetylation, methylation, glycosylation) in the isolated immune cell. In some embodiments, the method further comprises generating a report comprising information about the cancer status in the individual. In some embodiments, the information about cancer status comprises: classification of cancer; type of cancer; nature of cancer; origin of cancer; stage of cancer; likelihood of cancer progression; likelihood of developing one or more cancer symptoms; molecular diagnosis; NGS pathology; and/or treatment options for the individual. In some embodiments, the bait composition comprises a plurality of different display moieties. In some embodiments, each of the plurality of different display moieties in the bait composition comprises a different neoantigenic peptide (e.g., at least about two, four, 10, 25, 50, 75, or 100 different display moieties, each comprising a different MHC molecule). In some embodiments, each of the different display moieties comprising different MHC molecules comprises a different detectable label (e.g., a fluorophore). In some embodiments, the isolating step comprises using fluorescence-activated cell sorting (FACS), and/or separating immune cells associated with each of the different display moieties comprising different MHC molecules into different populations, optionally further comprises contacting each of a plurality of different display moieties with a sample from the individual separately and isolating the immune cell associated with each of the different display moiety. In some embodiments, the method further comprises culturing the isolated immune cell prior to the analyzing step. In some embodiments, the display moiety comprises a particle (e.g., a particle selected from the group consisting of: a surface, a nanoparticle, a bead, and a polymer). In some embodiments, the display moiety further comprises a detectable label. In some embodiments, the detectable label is a fluorophore. In some embodiments, analyzing the sequences of the one or more nucleic acids comprises whole genome sequencing, RNAseq sequencing, and/or subjecting the isolated immune cell to mass spectrometry analysis.

In some embodiments, the anti-cancer therapy is a standard or commonly used agent or therapy for treating cancer (e.g., a specific cancer). In some embodiments, the anti-cancer therapy comprises a chemotherapeutic agent. In some embodiments, the anti-cancer therapy comprises a surgery. In some embodiments, the anti-cancer therapy comprises a radiation therapy. In some embodiments, the anti-cancer therapy comprises an immunotherapy. In some embodiments, the anti-cancer therapy comprises a cell therapy (such as a cell therapy comprising an immune cell (e.g., CAR T cell)). In some embodiments, the anti-cancer therapy comprises an angiogenesis inhibitor.

In some embodiments, the anti-cancer therapy is not an immunotherapy.

In some embodiments, the individual has minimal residual disease (MRD). In some embodiments, the individual has minimal residual cancer. In some embodiments, the minimal residual cancer is seen after the cancer was surgical resected or cured. In some embodiments, the minimal residual disease is too minimal to be detected by imaging instruments (e.g., a routinely used or standard imaging instrument used for detection of the cancer). In some embodiments, the location of the minimal residual disease is diverse. In some embodiments, the minimal residual cancer is a result of immune escape or resistance to treatment. In some embodiments, the individual has been previously treated for cancer and exhibits no pathological symptom of a cancer after the treatment.

### Bait Compositions, Display Moieties and Neoantigen Peptides

In some embodiments, bait compositions described herein comprise one or more display moieties comprising one or more neoantigenic peptides, wherein an immune cell (such as a T cell) binds (e.g., cognately binds) to the display moiety and/or neoantigenic peptide.

In some embodiments, the display moiety described herein comprises an MHC molecule complexed with a neoantigenic peptide (e.g. a truncal neoantigenic peptide).

In some embodiments, the MHC molecule is a MHC class I molecule.

In some embodiments, the MHC class I molecule is selected from the group consisting of HLA-A, HLA-B, HLA-C. and HLA-D. In some embodiments, the MHC class I molecule is selected from the group consisting of HLA-A, HLA-B, and HLA-C. In preferred embodiments, the MHC class I molecule is selected from the group consisting HLA-A * 24:02, HLA-A*11:01, HLA-A*02:01, and HLA-A*03:01. In preferred embodiments, the MHC class I molecule comprises multiple kinds of MHC class I molecules comprising HLA-A * 24:02, HLA-A*11:01, HLA-A*02:01, and HLA-A*03:01.

In some embodiments, the neoantigenic peptide complexed with MHC I molecule is about 8 to about 10 amino acids long. In some embodiments, the neoantigenic peptide is at least 8 (e.g., 8, 9, or 10) amino acids long.

In some embodiments, the MHC molecule is a recombinant MHC I molecule.

In some embodiments, the MHC molecule is a MHC class II molecule.

In some embodiments, the MHC class II molecule is selected from the group consisting of HLA-DR. HLA-DQ, and HLA-DF. In some embodiments, the MHC class II molecule is selected from the group consisting of HLA-DQ and HLA-DR. In some embodiments, the neoantigenic peptide that complexed with a MHC class II molecule is about 10 to about 20 amino acids long. In some embodiments, the neoantigenic peptide is at least 10 (e.g., 10, 11, 12, 13, 14, 15, 16, 17, 18, 19, or 20) amino acids long.

In some embodiments, the MHC molecule is a recombinant MHC II molecule.

In some embodiments, the MHC molecule comprise both a MHC I molecule and a MHC II molecule.

In preferred embodiments, the MHC molecule matches with at least one HLA type of the individual from where the sample is obtained. For example, a MHC I molecule comprising HLA-A*24:02 or HLA-A *11:01 is used when the individual has both HLA-A * 24:02 and HLA-A*11:01. Patient-specific NGS data from WGS, WES, or RNA-seq can be used to predict HLA types with computational tools such as Optiptype and Polysolver (polymorphic loci resolver). See e.g., Szolet et al., Bioinformatics 30, 3310-3316, e.g., Shukla et al., Nat. Biotechnol. 33, 1152-1158. Reads can be selected from the NGS data that potentially derived from the HLA region and then they can be fully aligned to a full-length genomic library of all known HLA alleles. See e.g., Nucleic Acids Res. 41. D1222-D1227.

In some embodiments, the MHC molecule is coupled with a chaperon molecule prior to being complexed to the neoantigenic peptide. See e.g., Overall et al., Nat Commun. 2020 Apr. 20; 11(1):1909.

In some embodiments, the display moiety comprises two or more (e.g., at least 2, 3, 4, 5, 6, 7, 8, 9, 10, 20, 30, 40, 50, 60, 70, 80, 90, 100, 1000, 10,000, or 100,000) neoantigenic peptides. In some embodiments, the display moiety comprises four neoantigenic peptides. In some embodiments, the two or more neoantigenic peptides in the display moiety are the same or similar. In some embodiments, the two or more neoantigenic peptides in the display moiety are distinct. In some embodiments, at least one of the two or more neoantigenic peptides is a truncal neoantigen peptide.

In some embodiments, the display moiety comprises an MHC/peptide tetramer. In some embodiments, MHC/peptide complexes are assembled into tetramers, comprising 1, 2, 3, or 4 MHC/peptide complexes bound to a display moiety. In some embodiments, the MHC/peptide tetramer further comprises a detectable label. The detectable label is a fluorophore, such as phycocrythrin (PE), allophycocyanin (APC) or any fluorophore known in the art.

In some embodiments, the MHC/peptide complex is assembled into a multimer (such as, dimer, trimer, tetramer, pentamer, hexamer, or high order multimer). In some embodiments, the multimer can comprise at least 2, 3, 4, 5, 6, 7, 8, 9, or 10 MHC/peptide complexes. In some embodiments, a high throughput peptide-MHC (pMHC) tetramer library is constructed. See e.g., Overall et al., Nat Commun. 2020 Apr. 20; 11(1):1909.

In some embodiments, the display moiety further comprises a barcode (e.g., a DNA barcode). In some embodiments, each of the one or more display moieties comprises a unique barcode (e.g., a unique DNA barcode). See e.g., Overall et al., Nat Commun. 2020 Apr. 20; 11(1):1909.

In some embodiments, the display moiety comprises a particle. In some embodiments, the particle is selected from the group consisting of a surface, a nanoparticle, a bead, and a polymer. In some embodiments, the particle is a magnetic nanoparticle, e.g. for isolation using a magnet. See e.g., Peng et al., Cell Rep. 2019 Sep. 3; 28(10):2728-2738.e7. In some embodiments, the magnetic particle comprises magnetic iron oxide. In some embodiments, the particle is a polystyrene nanoparticle, e.g., for isolation by gravity. In some embodiments, the particle is an agarose bead. In some embodiments, the particle is a sepharose bead. In some embodiments, the particle is a dextran particle. In some embodiments, the particle is a biotinylated dextran or a streptavidin-coated dextran.

In some embodiments, the particle is detectable. In some embodiments, the particle is fluorescent. In some embodiments, the particle is attached directly or indirectly to a fluorophore. In some embodiments, the particle is modified with an attachment moiety for attaching additional molecules.

In some embodiments, the neoantigenic peptide or MHC is directly attached to the particle. In some embodiments, the neoantigenic peptide or MHC is attached to the particle via a binding pair comprising a first binding component attached to the neoantigenic peptide and a second binding component bound to the particle. In some embodiments, the binding components are any suitable moieties known in the art (such as, thiol, maleimide, cyclodextrin, amine, adamantine, carboxy, azide, and alkyne).

In some embodiments, multiple display moieties (e.g., MHC/peptide complexes) are attached to a single particle.

In some embodiments, the display moiety comprises a cell (e.g., an antigen presenting cell, e.g., a dendritic cell, e.g., a macrophage). In some embodiments, the cell comprises a polynucleotide encoding the neoantigenic peptide (e.g., a truncal neoantigen peptide). In some embodiments, the polynucleotide encodes a plurality of neoantigenic peptides. In some embodiments, the plurality of neoantigenic peptides are displayed on the surface of the cell. In some embodiments, the plurality of neoantigenic peptides are displayed on the surface of the cell in complex with an MHC molecule.

In some embodiments, the cell is obtained from the individual. In some embodiments, the cell has at least one (or two) same HLA type as that of the individual. For example, if the individual has HLA-A*24:02, the cell in the display moiety also has HLA-A*24:02.

In some embodiments, the display moiety further comprises a detectable label. In some embodiments, the detectable label is a fluorophore. In some embodiments, the display moiety is itself fluorescent or is attached to a fluorophore directly or indirectly. In some embodiments, the fluorophore is a phycoerythrin (PE), allophycocyanin (APC) or any fluorophore known in the art.

### Multiple Display Moieties

In some embodiments, the bait composition comprises a plurality of different display moieties. Use of the plurality of different display moieties can facilitate or promote identification of heterogeneous neoantigenic specific immune cells (e.g., T cells). In some embodiments, multiple display moieties comprise at least 2, 3, or 4 kinds of MHC molecules. In some embodiments, multiple display moieties comprise at least two (e.g., 2, or 3, or 4) different kinds of MHC class I molecules, optionally the at least two (e.g., 2, 3, or 4) different kinds of MHC class I molecules are selected from the group consisting of HLA-A * 24:02, HLA-A*11:01, HLA-A*02:01, and HLA-A*03:01. In some embodiments, multiple display moieties comprise HLA-A * 24:02, HLA-A*11:01, HLA-A*02:01, and HLA-A*03:01.

In some embodiments, at least two of the plurality of different display moieties in the bait composition comprise different neoantigenic peptides. In some embodiments, each of the plurality of different display moieties in the bait composition comprises a different neoantigenic peptide. In some embodiments, the plurality of different display moieties comprise a number (e.g., about 10, 20, 30, 40, 50, 60, 70, 80, 90 or 100) of different neoantigenic peptides. In some embodiments, at least 30%, 40%, 50%, 60%, 70%, 80%, or 90% of neoantigenic peptides for a cancer (e.g., a specific cancer) in a neoantigen database are included in the number of different neoantigenic peptides. In some embodiments, at least 30%, 40%, 50%, 60%, 70%, 80%, or 90% of neoantigenic peptides for a specific kind of MHC molecule (e.g., HLA-A * 24:02, HLA-A*11:01, HLA-A*02:01, or HLA-A*03:01) for a cancer (e.g., a specific cancer) in a neoantigen database are included in the number of different neoantigenic peptides.

In some embodiments, the plurality of different display moieties comprise at least one kind of MHC molecule that is HLA-A*24:02, wherein HLA-A*24:02 is complexed with at least two different neoantigenic peptides. In some embodiments, HLA-A*24:02 is complexed with a number (e.g., about 10, 20, 30, 40, 50, 60, 70, 80, 90 or 100) of neoantigen peptides, optionally wherein at least 30%, 40%, 50%, 60%, 70%, 80%, or 90% neoantigenic peptides for a cancer (e.g., a specific cancer) in a neoantigen database are included in the number of different neoantigenic peptides. In some embodiments, the plurality of different display moieties further comprises HLA-A*11:01, optionally wherein HLA-A*11:01 is complexed with at least two different neoantigenic peptides. In some embodiments, HLA-A*11:01 is complexed with a number (e.g., about 10, 20, 30, 40, 50, 60, 70, 80, 90 or 100) of neoantigen peptides, optionally wherein at least 30%, 40%, 50%, 60%, 70%, 80%, or 90% neoantigenic peptides for HLA-A*11:01 for a cancer (e.g., a specific cancer) in a neoantigen database are included in the number of different neoantigenic peptides. In some embodiments, the plurality of different display moieties further comprises HLA-A*02:01, optionally wherein HLA-A*02:01 is complexed with at least two different neoantigenic peptides. In some embodiments, HLA-A*02:01 is complexed with a number (e.g., about 10, 20, 30, 40, 50, 60, 70, 80, 90 or 100) of neoantigen peptides, optionally wherein at least 30%, 40%, 50%, 60%, 70%, 80%, or 90% neoantigenic peptides for HLA-A*02:01 for a cancer (e.g., a specific cancer) in a neoantigen database are included in the number of different neoantigenic peptides. In some embodiments, the plurality of different display moieties further comprises HLA-A*03:01, optionally wherein HLA-A*03:01 is complexed with at least two different neoantigenic peptides. In some embodiments, HLA-A*03:01 is complexed with a number (e.g., about 10, 20, 30, 40, 50, 60, 70, 80, 90 or 100) of neoantigen peptides, optionally wherein at least 30%, 40%, 50%, 60%, 70%, 80%, or 90% neoantigenic peptides for HLA-A*03:01 for a cancer (e.g., a specific cancer) in a neoantigen database are included in the number of different neoantigenic peptides.

In some embodiments, the plurality of different display moieties comprise at least one kind of MHC molecule that is HLA-A*11:01, wherein HLA-A*11:01 is complexed with at least two different neoantigenic peptides. In some embodiments, HLA-A*11:01 is complexed with a number (e.g., about 10, 20, 30, 40, 50, 60, 70, 80, 90 or 100) of neoantigen peptides, optionally wherein at least 30%, 40%, 50%, 60%, 70%, 80%, or 90% neoantigenic peptides for HLA-A*11:01 for a cancer (e.g., a specific cancer) in a neoantigen database are included in the number of different neoantigenic peptides. In some embodiments, the plurality of different display moieties further comprises HLA-A*02:01, optionally wherein HLA-A*02:01 is complexed with at least two different neoantigenic peptides. In some embodiments, HLA-A*02:01 is complexed with a number (e.g., about 10, 20, 30, 40, 50, 60, 70, 80, 90 or 100) of neoantigen peptides, optionally wherein at least 30%, 40%, 50%, 60%, 70%, 80%, or 90% neoantigenic peptides for HLA-A*02:01 for a cancer (e.g., a specific cancer) in a neoantigen database are included in the number of different neoantigenic peptides. In some embodiments, the plurality of different display moieties further comprises HLA-A*03:01, optionally wherein HLA-A*03:01 is complexed with at least two different neoantigenic peptides. In some embodiments, HLA-A*03:01 is complexed with a number (e.g., about 10, 20, 30, 40, 50, 60, 70, 80, 90 or 100) of neoantigen peptides, optionally wherein at least 30%, 40%, 50%, 60%, 70%, 80%, or 90% neoantigenic peptides for HLA-A*03:01 for a cancer (e.g., a specific cancer) in a neoantigen database are included in the number of different neoantigenic peptides.

In some embodiments, the plurality of different display moieties comprise at least one kind of MHC molecule that is HLA-A*02:01, wherein HLA-A*02:01 is complexed with at least two different neoantigenic peptides. In some embodiments, HLA-A*02:01 is complexed with a number (e.g., about 10, 20, 30, 40, 50, 60, 70, 80, 90 or 100) of neoantigen peptides, optionally wherein at least 30%, 40%, 50%, 60%, 70%, 80%, or 90% neoantigenic peptides for HLA-A*02:01 for a cancer (e.g., a specific cancer) in a neoantigen database are included in the number of different neoantigenic peptides. In some embodiments, the plurality of different display moieties further comprises HLA-A*03:01, optionally wherein HLA-A*03:01 is complexed with at least two different neoantigenic peptides. In some embodiments, HLA-A*03:01 is complexed with a number (e.g., about 10, 20, 30, 40, 50, 60, 70, 80, 90 or 100) of neoantigen peptides, optionally wherein at least 30%, 40%, 50%, 60%, 70%, 80%, or 90% neoantigenic peptides for HLA-A*03:01 for a cancer (e.g., a specific cancer) in a neoantigen database are included in the number of different neoantigenic peptides.

In some embodiments, the plurality of different display moieties comprise at least one kind of MHC molecule that is HLA-A*03:01, wherein HLA-A*03:01 is complexed with at least two different neoantigenic peptides. In some embodiments, HLA-A*03:01 is complexed with a number (e.g., about 10, 20, 30, 40, 50, 60, 70, 80, 90 or 100) of neoantigen peptides, optionally wherein at least 30%, 40%, 50%, 60%, 70%, 80%, or 90% neoantigenic peptides for HLA-A*03:01 for a cancer (e.g., a specific cancer) in a neoantigen database are included in the number of different neoantigenic peptides.

In some embodiments, the plurality of different display moieties comprise at least four kinds of MHC molecules, wherein the at least four kinds of MHC molecules comprise HLA-A*24:02, HLA-A*11:01, HLA-A*02:01, and HLA-A*03:01. In some embodiments, each kind of the MHC molecules (HLA-A*24:02, HLA-A*11:01, HLA-A*02:01) are complexed with at least two different neoantigenic peptides. In some embodiments, each kind of the MHC molecule is complexed with a number (e.g., about 10, 20, 30, 40, 50, 60, 70, 80, 90 or 100) of neoantigen peptides, optionally wherein at least 30%, 40%, 50%, 60%, 70%, 80%, or 90% neoantigenic peptides for the specific MHC molecule for a cancer (e.g., a specific cancer) in a neoantigen database are included in the number of different neoantigenic peptides.

In some embodiments, the method comprises separately contacting each of a plurality of different display moieties with a sample from the individual separately and separately isolating the immune cell associated with each of the different display moiety.

In some embodiments, the method comprises contacting the plurality of different display moieties with a sample from the individual, and analyzing the pool of immune cells. For example, use of multi-color-labeled MHC tetramers for multiplex flow cytometry, pMHC tetramers labeled for mass cytometry analysis, and DNA-labeled tetramers designed for sequencing analysis have also been reported. See e.g., Andersen et al. Nat Protoc. 2012 Apr. 12; 7(5):891-902.

### Neoantigenic Peptides

Neoantigens can be formed via various mechanisms. Non-synonymous somatic mutations, which can alter amino acid coding sequences, are the main cause of neo-epitopes. Except for somatic non-synonymous protein-altering mutations, tumor neoantigens can be generated from alternative splicing variations. Multiple computational methods and databases have been developed to identify alternative splicing events from RNA-seq data, such as SplAdder and CancerSplicingQTL2. See e.g., Kahles et al., Bioinformatics 32 1840-1847; Tian et al., Nucleic Acids Res. 47 D909-D916. A computational strategy was developed to identify neoepitopes generated from intron retention events in tumor transcriptomes and confirmed that these neoepitopes were processed and presented on MHC-I. See Smart et al. Nat. Biotechnol. 36 1056-1058.

### Detection and Screening of Neoantigens

Various methods are available to detect and screen neoantigens. Sandwich immunoassays in the miniaturized system could successfully identify tumor antigens in sera samples extracted from patients. See e.g., Pollard et al., Proteomics Clin. Appl. 1 934-952 (2007); Yang et al., Biosens. Bioelectron. 40 385-392 (2013). Another tool named Serologic Proteome analysis (SERPA) or 2-D western blots, consists of the isoelectric focusing (IEF) gel run in the first dimension and SDS-PAGE gel run in the second dimension. SERPA separates the proteins in the gel by their isoelectric point (IP) and molecular mass and then transfers the proteins from the gel to a carrier membrane to screen antibodies. Finally, the antigenic protein spots can be identified by MS. See e.g., Tjalsma et al., Proteomics Clin. Appl. 2 167-180 (2008). This approach has been used to identify antigens in different tumor types. Serological analysis of recombinant cDNA expression libraries (SEREX), which combines serological analysis with antigen cloning techniques, is a widely used technique to explore tumors' antigen repertoire. SEREX first construct a cDNA library from cancer cell lines or fresh tumor samples, then screen the cDNA library with autologous sera of cancer patients, and finally sequence the immune-reactive clones. SEREX have identified a variety of tumor antigens including CTAs, differentiation antigens, mutational antigens, splice-variant antigens and overexpressed antigens. See e.g., Chen et al., Proc. Natl. Acad. Sci. U.S.A. 94 1914-1918 (1997). Furthermore, other methods such as Multiple Affinity Protein Profiling (MAPPing) and nanoplasmonic biosensor have also been developed to identify tumor antigens. See e.g., Lee et al., Biosens. Bioelectron. 74 341-346 (2015).

In some embodiments, the one or more neoantigenic peptides described herein are obtained from a neoantigenic database (such as any of the neoantigenic databases described herein). For example, Tan et al constructed a manually curated database (“dbPepNeo”) for human tumor neoantigen peptides based upon the four criterias as below: (i) peptides were isolated from human tumor tissues or cell lines, (ii) peptides contained non-synonymous mutations in amino acid sequence, (iii) Peptides can be bound by HLA-I molecules, (iv) Peptides can induce CD8+ T cell responses. See Tan et al., Database (Oxford). 2020 Jan. 1; 2020:baaa004. Xia et al constructed another database, NEPdb, which provides pan-cancer level predicted HLA-I neoepitopes derived from 16,745 shared cancer somatic mutations, using state-of-the-art predictors. See Xia et al., Front Immunol. 2021; 12:644637. Wu et al. developed a comprehensive tumor-specific neoantigen database (TSNAdb v1.0), based on pan-cancer immunogenomic analyses of somatic mutation data and human leukocyte antigen (HLA) allele information for 16 tumor types with 7748 tumor samples from The Cancer Genome Atlas (TCGA) and The Cancer Immunome Atlas (TCIA). See Wu et al., enomics Proteomics Bioinformatics. 2018 August; 16 (4): 276-282.

In some embodiments, the one or more neoantigenic peptides are obtained from analyzing the biological information of the individual (such as a patient who had a cancer). In some embodiments, the neoantigenic peptides are obtained from a computational analysis of a cancer patient's tumor genome. See e.g., Roudko et al. Front Immunol. 2020; 11:27. In some embodiments, the neoantigenic peptides are obtained from a computational analysis of a cancer patient's transcriptome. See e.g., Caushi et al., Nature. 2021 August; 596(7870):126-132. In some embodiments, the neoantigenic peptides are obtained from a computational analysis of a cancer patient's proteome. See e.g., Wen et al. Nat Commun. 2020 Apr. 9; 11(1):1759.

In some embodiments, the neoantigenic peptides are selected from patient data. In some embodiments, the patient data is derived from data from a group of patients having a particular type of cancer (e.g., any of the cancers described here). In some embodiments, the patient data is derived from data from a group of patients having any cancer. In some embodiments, the group of patients are from the same sex (e.g., male or female). In some embodiments, the group of patients are from the same ethnicity. In some embodiments, the group of patients bear one or more biomarkers (e.g., an aberration in a particular gene, e.g., KRAS, e.g., PTEN).

In some embodiments, the one or more neoantigenic peptides are derived from any polypeptide known to or have been found to contain a tumor specific mutation. Suitable polypeptides from which the neoantigenic peptides can be derived can be found for example in various databases available in the field (e.g., COSMIC database). These databases curate comprehensive information on somatic mutations in human cancer. In some embodiments, the peptide contains a tumor specific mutation. In some embodiments, the tumor specific mutation is a driver mutation for a particular cancer type.

In some embodiments, a library of tumor specific neoantigenic peptides are synthesized (e.g., based upon one or a group of patients as described above). In some embodiments, the neoantigenic peptides are obtained by exome high throughput sequencing and prescreened with epitope prediction algorithms.

### Selecting Neoantigenic Peptides

In some embodiments, the one or more neoantigenic peptides used in the bait compositions described herein are further optimized based upon one or more selection criteria.

In some embodiments, the neoantigenic peptides are further selected based on their likelihood to be processed and/or presented on the cell surface HLA molecules. In some embodiments, in silico prediction algorithms (such as any of the algorithms described herein) is used as the basis for the selection. In some embodiments, immunopeptidomics analysis is used as the basis for the selection.

Computational algorithms such as NetMHC (See e.g., Andreatta et al., Bioinformatics 32, 511-517, 2016), NetMHCpan (See e.g., Rammensce et al., Immunogenetics 50, 213-219, 1999), and MHCflurry (O'Donnell et al., Cell Syst. 7, 129-132.e124, 2018) trained on large in vitro experimental datasets can be used to prioritize candidate neoantigens that bind to the predicted HLA types with high affinity. For example, Neopepsee and pVAC-Seq are representative analysis pipelines for tumor somatic mutations (Hundal et al., Genome Med. 8, 11, 2016; Kim et al., Ann. Oncol. 29, 1030-1036, 2018). Recently, a new prediction model-EDGE based on tumor HLA peptide mass spectrometry (MS) datasets has increased the positive predictive value up to nine-fold. (Bulik-Sullivan et al., Nat. Biotechnol. 18:4313).

In some embodiments, the one or more neoantigenic peptides are selected based upon its binding affinity to a) an MHC molecule and/or b) a cognate TCR molecule.

In some embodiments, the neoantigenic peptide has a binding affinity that is less than 5000 nM (IC50) to an MHC molecule. In some embodiments, the neoantigenic peptide has a binding affinity of about 500 nM to 5000 nM (IC50) to an MHC molecule. In some embodiments, the neoantigenic peptide has a binding affinity that is less than 500 nM (IC50) to an MHC molecule. In some embodiments, the neoantigenic peptide has a binding affinity of about 250 nM to 500 nM IC50 to an MHC molecule. In some embodiments, the neoantigenic peptide has a binding affinity that is less than 250 nM (IC50) to an MHC molecule. In some embodiments, the neoantigenic peptide has a binding affinity that is less than 100 nM (IC50) to an MHC molecule. In some embodiments, the neoantigenic peptide has a binding affinity of about 50 nM to 500 nM IC50 to an MHC molecule. In some embodiments, the neoantigenic peptide has a binding affinity that is less than 50 nM (IC50) to an MHC molecule. In some embodiments, the neoantigenic peptide has a binding affinity of about 1 nM to 50 nM IC50 to an MHC molecule.

In some embodiments, the neoantigenic peptide has a binding affinity that is less than 5000 nM (IC50) to a cognate TCR molecule. In some embodiments, the neoantigenic peptide has a binding affinity of about 500 nM to 5000 nM (IC50) to a cognate TCR molecule. In some embodiments, the neoantigenic peptide has a binding affinity of about 50 nM to 500 nM IC50 to a cognate TCR molecule. In some embodiments, the neoantigenic peptide has a binding affinity of about 1 nM to 50 nM IC50 to a cognate TCR molecule.

In some embodiments, the neoantigenic peptide is selected based upon its mutational status. In some embodiments, the neoantigenic peptide has a mutation at the third amino acid position counting from the N-terminus, relative to a wildtype peptide. In some embodiments, the neoantigenic peptides may comprise two or more (such as at least 2, 3, 4, or 5) somatic mutations.

In some embodiments, the neoantigenic peptide is selected based upon its hydrophobic status. In some embodiments, the neoantigenic peptide is hydrophobic. In some embodiments, the neoantigenic peptide has a high content of aromatic residues. In some embodiments, the neoantigenic peptide has at least about 10%, 20%, 30%, or 40% aromatic residues.

In some embodiments, the neoantigenic peptide has a binding affinity of about 1 nM to about 5000 nM (e.g., about 1 nM to about 50 nM, about 50 nM to about 500 nM, about 500 nM to about 5000 nM) to an MHC molecule, a binding affinity of about 1 nM to about 5000 nM (e.g., about 1 nM to about 50 nM, about 50 nM to about 500 nM, about 500 nM to about 5000 nM) to a cognate TCR molecule, a mutation relative to a wildtype peptide, optionally at the third amino acid position counting from the N-terminus, is hydrophobic, and has high content of aromatic residues.

In some embodiments, the neoantigenic peptide has low immunogenicity. Immunogenicity of the neoantigenic peptide can be predicted by algorithm developed for this purpose. See e.g., Riley et al., Front Immunol. 2019 Aug. 28; 10:2047; e.g., Schmidt et al., Cell Rep Med. 2021 Feb. 6; 2(2):100194.

In some embodiments, the cancer antigenic peptide may be flanked by universal sequences or portions thereof. In some embodiments, the universal sequences or portions allow for rapid, high throughput methods for replacing or inserting the antigenic peptide encoding nucleotide in the polynucleotide MHC template.

In some embodiments, the neoantigenic peptides further comprise a unique defined barcode sequence operably associated with the identity of each distinct polypeptide. In some embodiments, the unique defined barcodes provide an antigen-specific sequence for identification during the analysis of the immune cell. See e.g., Peng et al., Cell Rep. 2019 Sep. 3; 28(10):2728-2738.e7.

The size of a neoantigenic peptide can comprise, but is not limited to, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 60, about 70, about 80, about 90, about 100, about 110, about 120 or greater amino molecule residues, and any range derivable therein. In preferred embodiments, the neoantigenic peptide molecules are equal to or less than 50 amino acids.

In some embodiments, the neoantigenic peptide complexed with a MHC Class I molecule has 15 residues or less in length and usually consist of between about 8 and about 11 residues, particularly 9 or 10 residues. In some embodiments, the neoantigenic peptides complexed with a MHC Class II molecule has 6-30 residues.

If desirable, a longer peptide can be designed in several ways. For example, when presentation likelihoods of peptides on HLA alleles are predicted or known, a longer peptide could consist of either: (1) individual presented peptides with an extensions of 2-5 amino acids toward the N- and C-terminus of each corresponding gene product; (2) a concatenation of some or all of the presented peptides with extended sequences for each.

In some embodiments, the one or more neoantigenic peptides are about 8-50 amino acids in length. In some embodiments, it is about 8-10 amino acids in length. In some embodiments, it is greater than 10 amino acids in length, greater than 15 amino acids in length, greater than 20 amino acids in length, or greater than 30 amino acids in length. In some embodiments, it is about 24-40 amino acids in length.

In some embodiments, the methods involve constructing a plurality of neoantigen peptide peptides (e.g., building a library, such as in Example 1 and FIG. 1). In some embodiments, the plurality of neoantigen peptides are based upon a known mutation (e.g., Kras mutation). In some embodiments, the plurality of neoantigen peptides based upon one known mutation (e.g., a point mutation, e.g., Kras G12D, G12V, G12C, G12R, G12A, or G12I) comprises at least about 5, 10, 12, 15, 18 or 20 distinct neoantigen peptides. In some embodiments, the plurality of neoantigen peptides based upon one known mutation comprise at least about 5, 10, 12, 15, 18 or 20 distinct neoantigen peptides that a) have a binding affinity of about 1 nM to about 5000 nM to an MHC molecule; b) have a binding affinity of about 1 nM to about 5000 nM to a cognate TCR molecule; c) have a mutation relative to a wildtype peptide, optionally at the third amino acid position counting from the N-terminus; d) are hydrophobic; and/or e) have high content of aromatic residues.

### Exemplary Considerations for Bait Composition Design

In some embodiments, the one or more neoantigenic peptides are selected through one or more steps as described below.

1. Determination of a Set of Peptides that Cover an Optimized Number of Tumor Subclones

In some embodiments, the one or more neoantigenic peptides comprise a truncal peptide. Truncal peptides described herein refer to those presented by all or most tumor subclones, is prioritized for inclusion into the bait composition.

If there are no truncal peptides predicted to be presented with high probability, or if the number of truncal peptides predicted to be presented with high probability is small enough that additional non-truncal peptides can be included in the display moiety, then further peptides can be prioritized by estimating the number and identity of tumor subclones and choosing peptides so as to maximize the number of tumor subclones covered.

### 2. Neoantigen Selection

Neoantigenic peptides can be selected via various methods or processes. In some embodiments, an integrated multi-dimensional model described below is applied that places candidate neoantigens in a space with at least one or more of the following axes and optimizes selection using an integrative approach. See e.g., WO2019050994A.

1. Probability of sequencing artifact (lower probability of artifact is typically preferred).

2. Probability of presentation (higher probability of presentation is typically preferred).

3. Gene expression (higher expression is typically preferred).

4. Coverage of HLA genes (larger number of HLA molecules involved in the presentation of a set of neoantigens is typically preferred).

5. Coverage of both MHC classes (covering both MHC-I and MHC-II is preferred).

Exemplary Bait Compositions, Display Moieties and Neoantigens (e.g., for Analyzing Immune Cells from an Individual for the Presence of a Lung Cancer (e.g., NSCLC))

Exemplary bait compositions were determined using the methodology described herein.

TABLE 1
Exemplary neoantigenic peptides

| SEQ ID NO | HLA type | sequence |
|---|---|---|
| 1 | HLA-A*24:02 | VYCEEYYLF |
| 2 | HLA-A*02:01 | YQANVVWKV |
| 3 | HLA-A*03:01 | ALYFNSQWK |
| 4 | HLA-A*02:01 | KLLSFHSV |
| 5 | HLA-A*02:01 | YLNEAVFNFV |
| 6 | HLA-A*24:02 | FYMHEYPEGW |
| 7 | HLA-A*01:01 | EIDLPRELEY |
| 8 | HLA-A*02:01 | KQDGYDSV |
| 9 | HLA-A*11:01 | LLQHYLLYR |
| 10 | HLA-A*11:01 | TTARMRTMR |
| 11 | HLA-A*11:01 | HTFHLQDHH |
| 12 | HLA-A*11:01 | STTGATDLK |
| 13 | HLA-A*11:01 | MTFAETYPA |
| 14 | HLA-A*03:01 | NLLLIRGFK |
| 15 | HLA-A*11:01 | HGYFWFMGR |
| 16 | HLA-A*11:01 | FVLAALMEY |
| 17 | HLA-A*03:01 | RLRRLPVPR |
| 18 | HLA-A*11:01 | QSLVPAHPK |
| 19 | HLA-A*11:01 | RSFLSWDSR |
| 20 | HLA-A*03:01 | RIQGYIIEK |
| 21 | HLA-A*03:01 | ALNGMPLLK |
| 22 | HLA-A*11:01 | GTSESRETR |
| 23 | HLA-A*24:02 | IFSHPLYNI |
| 24 | HLA-A*11:01 | AAYLLFYQR |
| 25 | HLA-A*24:02 | SYVNILRAI |
| 26 | HLA-A*11:01 | RVEPVNYPK |
| 27 | HLA-A*24:02 | GYMGQQNEL |
| 28 | HLA-A*11:01 | RTQEARPPR |
| 29 | HLA-A*11:01 | RTASEDHPR |
| 30 | HLA-A*11:01 | VTIFVYDVK |
| 31 | HLA-A*11:01 | VVCIDAFLK |
| 32 | HLA-A*11:01 | STDQPVIPK |
| 33 | HLA-A*11:01 | SVAWPQDRR |
| 34 | HLA-A*11:01 | PTFGLSILK |
| 35 | HLA-A*24:02 | SYGLILLAF |
| 36 | HLA-A*24:02 | VFPLLFGTF |
| 37 | HLA-A*24:02 | YYLCLRHRL |
| 38 | HLA-A*11:01 | TSFPLDANK |
| 39 | HLA-A*11:01 | SLMVCNHDK |
| 40 | HLA-A*24:02 | SYIYILIII |
| 41 | HLA-A*11:01 | GVFRRCWEK |
| 42 | HLA-A*11:01 | RSAAIASEK |
| 43 | HLA-A*11:01 | SSTHPHFVR |
| 44 | HLA-A*11:01 | NVLEINFIK |
| 45 | HLA-A*11:01 | SMVPVMYQK |
| 46 | HLA-A*11:01 | SVYCIGQRR |
| 47 | HLA-A*11:01 | YQMLSFVHK |
| 48 | HLA-A*24:02 | LYSFISRFI |
| 49 | HLA-A*11:01 | FSFRSCNFK |
| 50 | HLA-A*11:01 | ISYAKYFPK |
| 51 | HLA-B*44:02 | AEYQDMHSY |
| 52 | HLA-B*44:02 | VEHINISQDW |
| 53 | HLA-B*07:02 | RGRMQTASL |
| 54 | HLA-C*06:02 | VRINTARPV |
| 55 | HLA-B*27:05 | RRSMLFARH |
| 56 | HLA-C*05:01 | KTDTGVHATL |

In some embodiments, the one or more neoantigenic peptides comprise one or more (e.g., at least 2, 5, 10, 15, 20, 25, 30, 35, 40, 45, or 50) sequences selected from the group consisting of SEQ ID NOs: 1-56.

In some embodiments, the one or more neoantigenic peptides comprise the amino acid sequences set forth in SEQ ID NOs: 1-5. In some embodiments, the one or more neoantigenic peptides further comprise one or more (e.g., at least 2, 5, 10, 15, 20, 25, 30, 35, or 40) sequences selected from the group consisting of SEQ ID NOs: 6-50 and/or one or more (e.g., 1, 2, 3, 4, 5 or 6) sequences selected from the group consisting of SEQ ID NOs: 51-56.

In some embodiments, the one or more neoantigenic peptides comprise one or more (e.g., at least 2, 5, 10, 15, 20, 25, 30, 35, or 40) sequences selected from the group consisting of SEQ ID NOs: 6-50. In some embodiments, the one or more neoantigenic peptides comprise the amino acid sequences set forth in SEQ ID NOs: 6-50. In some embodiments, the one or more neoantigenic peptides further comprise one or more (e.g., 1, 2, 3, 4, or 5) sequences selected from the group consisting of SEQ ID NOs: 1-5 and/or one or more (e.g., 1, 2, 3, 4, 5 or 6) sequences selected from the group consisting of SEQ ID NOs: 51-56.

In some embodiments, the one or more neoantigenic peptides comprise the amino acid sequences set forth in SEQ ID NOs: 51-56. In some embodiments, the one or more neoantigenic peptides further comprise one or more (e.g., at least 2, 5, 10, 15, 20, 25, 30, 35, or 40) sequences selected from the group consisting of SEQ ID NOs: 6-50 and/or one or more one or more (e.g., 1, 2, 3, 4, or 5) sequences selected from the group consisting of SEQ ID NOs: 1-5.

In some embodiments, the one or more (e.g., 1, 2, 3, 4, 5, 6, 7, 8, 9, or 10) neoantigenic peptides comprise the amino acid sequences selected from the group consisting of SEQ ID NOs: 1, 6, 23, 25, 27, 35-37, 40, and 48.

In some embodiments, the one or more (e.g., 5, 10, 15, 20, 25, or 30) neoantigenic peptides comprise the amino acid sequences selected from the group consisting of SEQ ID NOs: 9-13, 15, 16, 18, 19, 22, 24, 26, 28-34, 38, 39, 41-47, 49, and 50.

In some embodiments, the one or more (e.g., 1, 2, 3, or 4) neoantigenic peptides comprise the amino acid sequences selected from the group consisting of SEQ ID NOs: 2, 4, 5, and 8.

In some embodiments, the one or more (e.g., 1, 2, 3, 4, or 5) neoantigenic peptides comprise the amino acid sequences selected from the group consisting of SEQ ID NOs: 3, 14, 17, 20, and 21.

In some embodiments, the one or more (e.g., 1, 2, 3, or 4) neoantigenic peptides comprise the amino acid sequences selected from the group consisting of SEQ ID NOs: 53, 55, 51, and 52.

In some embodiments, the one or more (e.g., 1 or 2) neoantigenic peptides comprise the amino acid sequences selected from the group consisting of SEQ ID NOs: 54 and 56.

In some embodiments, the one or more neoantigenic peptides comprise amino acid sequences set forth in SEQ ID NOs: 1-56.

In some embodiments, the bait composition comprises one or more display moieties comprising one or more (e.g., at least 2, 5, 10, 15, 20, 25, 30, 35, 40, 45, or 50) MHC-peptide complexes (i.e., pMHC) paired as shown in Table 1. In some embodiments, the bait composition comprises one or more display moieties comprising pMHC comprising one or more neoantigenic peptides set forth in SEQ ID NOs 1-5 complexed with a MHC molecule according to Table 1. In some embodiments, the bait composition comprises one or more display moieties comprising pMHC comprising one or more (e.g., at least 2, 5, 10, 15, 20, 25, 30, 35, or 40) neoantigenic peptides comprising amino acids selected from the group consisting of SEQ ID NOs: 6-50 complexed with a MHC molecule according to Table 1. In some embodiments, the bait composition comprises one or more display moieties comprising pMHC comprising one or more (e.g., 1, 2, 3, 4, 5 or 6) neoantigenic peptides comprising amino acids selected from the group consisting of SEQ ID NOs: 51-56 complexed with a MHC molecule according to Table 1.

In some embodiments, the display moieties comprise one or more MHC-peptide complexes comprising neoantigenic peptides comprising one or more (e.g., 1, 2, 3, 4, 5, 6, 7, 8, 9, or 10) amino acid sequences selected from the group consisting of SEQ ID NOs: 1, 6, 23, 25, 27, 35-37, 40, and 48, wherein the MHC molecule is HLA-A*24:02. In some embodiments, the display moieties comprise one or more MHC-peptide complexes comprising neoantigenic peptides comprising amino acid sequences set forth in SEQ ID NOS: 1, 6, 23, 25, 27, 35-37, 40, and 48, wherein the MHC molecule is HLA-A*24:02.

In some embodiments, the display moieties comprise one or more MHC-peptide complexes comprising neoantigenic peptides comprising (e.g., 5, 10, 15, 20, 25, or 30) amino acid sequences selected from the group consisting of SEQ ID NOs: 9-13, 15, 16, 18, 19, 22, 24, 26, 28-34, 38, 39, 41-47, 49, and 50, wherein the MHC molecule is HLA-A*11:01. In some embodiments, the display moieties comprise one or more MHC-peptide complexes comprising neoantigenic peptides comprising amino acid sequences set forth in SEQ ID NOS: 9-13, 15, 16, 18, 19, 22, 24, 26, 28-34, 38, 39, 41-47, 49, and 50, wherein the MHC molecule is HLA-A*11:01.

In some embodiments, the display moieties comprise one or more MHC-peptide complexes comprising neoantigenic peptides comprising one or more (e.g., 1, 2, 3, or 4) amino acid sequences selected from the group consisting of SEQ ID NOs: 2, 4, 5, and 8, wherein the MHC molecule is HLA-A*02:01. In some embodiments, the display moieties comprise one or more MHC-peptide complexes comprising neoantigenic peptides comprising amino acid sequences set forth in SEQ ID NOs: 2, 4, 5, and 8, wherein the MHC molecule is HLA-A*02:01.

In some embodiments, the display moieties comprise one or more MHC-peptide complexes comprising neoantigenic peptides comprising one or more (e.g., 1, 2, 3, 4 or 5) amino acid sequences selected from the group consisting of SEQ ID NOs: 3, 14, 17, 20, and 21, wherein the MHC molecule is HLA-A*03:01. In some embodiments, the display moieties comprise one or more MHC-peptide complexes comprising neoantigenic peptides comprising amino acid sequences set forth in SEQ ID NOs: 3, 14, 17, 20, and 21, wherein the MHC molecule is HLA-A*03:01.

In some embodiments, the display moieties comprise one or more MHC-peptide complexes comprising neoantigenic peptides comprising one or more (e.g., 1, 2, 3, or 4) amino acid sequences selected from the group consisting of SEQ ID NOs: 53, 55, 51, and 52, wherein the MHC molecule is HLA-B molecule. In some embodiments, the display moieties comprise one or more MHC-peptide complexes comprising neoantigenic peptides comprising amino acid sequences set forth in SEQ ID NOs: 53, 55, 51, and 52, wherein the MHC molecule is HLA-B molecule.

In some embodiments, the display moieties comprise one or more MHC-peptide complexes comprising neoantigenic peptides comprising one or more amino acid sequences selected from the group consisting of SEQ ID NOs: 54 and 56, wherein the MHC molecule is HLA-C molecule. In some embodiments, the display moieties comprise one or more MHC-peptide complexes comprising neoantigenic peptides comprising amino acid sequences set forth in SEQ ID NOs: 54 and 56, wherein the MHC molecule is HLA-C molecule.

In some embodiments, the display moieties comprise MHC-peptide complexes comprising neoantigenic peptides comprising amino acid sequences set forth in SEQ ID NO: 1-56, wherein each of the neoantigen peptides is complexed with a MHC molecule according to Table 1.

### Modifications of Neoantigenic Peptides

Neoantigenic peptides having a desired activity or property can be modified to provide certain desired attributes, while increasing or at least retaining substantially all of the biological activity of the unmodified peptide to bind the desired MHC molecule and activate the appropriate immune cell (e.g., a T cell). For instance, neoantigenic peptide described herein can be subject to various changes, such as substitutions, either conservative or non-conservative, where such changes might provide for certain advantages in their use, such as improved MHC binding, stability or presentation. By conservative substitutions is meant replacing an amino acid residue with another which is biologically and/or chemically similar, e.g., one hydrophobic residue for another, or one polar residue for another. The substitutions include combinations such as Gly, Ala; Val, Ile, Leu, Met; Asp, Glu; Asn, Gin; Ser, Thr; Lys, Arg; and Phe, Tyr. The effect of single amino acid substitutions may also be probed using D-amino acids. Such modifications can be made using well known peptide synthesis procedures, as described in e.g., Merrifield, Science 232:341-347 (1986), Barany & Merrifield, The Peptides, Gross & Meienhofer, eds. (N.Y., Academic Press), pp. 1-284 (1979); and Stewart & Young, Solid Phase Peptide Synthesis, (Rockford, 111., Pierce), 2d Ed. (1984).

Proteins or peptides described herein can be made by any technique known to those of skill in the art, including the expression of proteins, polypeptides or peptides through standard molecular biological techniques, the isolation of proteins or peptides from natural sources, or the chemical synthesis of proteins or peptides. The nucleotide and protein, polypeptide and peptide sequences corresponding to various genes have been previously disclosed, and can be found at computerized databases known to those of ordinary skill in the art. One such database is the National Center for Biotechnology Information's Genbank and GenPept databases located at the National Institutes of Health website. The coding regions for known genes can be amplified and/or expressed using the techniques disclosed herein or as would be known to those of ordinary skill in the art. Alternatively, various commercial preparations of proteins, polypeptides and peptides are known to those of skill in the art.

Isolating, Culturing, and/or Analyzing the Immune Cells that Bind to the Display Moiety

In some embodiments, the methods described herein comprise isolating an immune cell associated with the display moiety; and analyzing the isolated immune cell. In some embodiments, the methods described herein further provides culturing the isolated immune cell prior to the analyzing step.

In some embodiments, the methods described herein does not comprise a step of culturing the isolated immune cell prior to the analyzing step.

In some embodiments, the isolating step comprises separating immune cells associated with the display moiety from the rest of the sample.

In some embodiments, the isolating step comprises using fluorescence-activated cell sorting (FACS). In some embodiments, the display moiety may be attached to one or more fluorescent attachment moieties, such as a streptavidin core attached or bound to a fluorescent molecule. In some embodiments, the display moiety is fluorescent or conjugated to a fluorophore directly. In some embodiments, multiple elements within the display moiety (such as the particle, attachment moiety, binding component) can be fluorescent, including each comprising a different fluorophore. In some embodiments, the display moiety used is magnetic or non-magnetic. In some embodiments, magnetic separation methods can be used in conjunction with FACS (e.g., before, after, or before and after FACS). In some embodiments, magnetic activated cell sorting, affinity chromatography, or any of the methods of cell sorting known in the art are used.

In some embodiments, the isolated immune cell is selected from the group consisting of: a cytotoxic T cell (e.g., a CD8+ T cell), a helper T cell (e.g., a CD4+ T cell), a memory T cell, and a tumor infiltrating T cell. In some embodiments, the isolated immune cell is a helper T cell.

In some embodiments, the isolated immune cell is a B cell. In some embodiments, the B cell is a memory B cell.

In some embodiments, the isolated immune cell is an isolated single immune cell. In some embodiments, the isolated immune cell is in a mixture of immune cells. For example, the isolated immune cell (e.g., a T cell) can be in a mixture of immune cells (e.g., a mixture containing both T cells and antigen presenting cells) when contacting with the display moiety. In some embodiments, the mixture of immune cells is a mixture comprising T cells, memory T cells, macrophage cells, or dendritic cells, or combinations thereof.

In some embodiments, culturing the isolated immune cell comprises incubating the isolated immune cell (such as a T cell) with one or more neoantigenic peptides described herein. In some embodiments, culturing the isolated immune cell (such as a T cell) comprising incubating the isolated immune cell (such as a T cell) with one or more neoantigenic peptides described herein for at least 1, 2, 3, 4, 5, 6, 7, 8, 9, 10, 11, 12, 13, 14, 15 days. In some embodiments, culturing the isolated immune cell (such as a T cell) comprising incubating the isolated immune cell (such as a T cell) with one or more neoantigenic peptides described herein for less than 5, 4, 3, 2, or 1 day. In some embodiments, culturing the isolated immune cell comprises incubating the isolated immune cell (such as a T cell) with one or more cytokines (e.g., IL-2, IL-7, IL-15).

In some embodiments, analyzing the isolated immune cell comprises detecting the isolated immune cell. In some embodiments, analyzing the isolated immune cell comprises quantifying the isolated immune cell. In some embodiments, analyzing the isolated immune cell comprises sequencing one or more nucleic acids in the isolated immune cell. In some embodiments, analyzing the isolated immune cell further comprises analyzing the sequences of the one or more nucleic acids. In some embodiments, the one or more nucleic acids comprises a nucleic acid sequence selected from the group consisting of a TCR sequence. In some embodiments, analyzing the sequences of the one or more nucleic acids comprises whole genome sequencing. In some embodiments, analyzing the sequences of the one or more nucleic acids comprises RNA sequencing. In some embodiments, analyzing the sequences of the one or more nucleic acids comprises single cell sequencing (such as scRNAseq). In some embodiments, analyzing the sequences of the one or more nucleic acids comprises decoding the barcode sequences. In some embodiments, analyzing the sequences of the one or more nucleic acids comprises identifying the rearranged genes on the tumor specific neoantigen activated immune cells. In some embodiments, analyzing the sequences of the one or more nucleic acids comprises identifying the rearranged T cell receptor (TCR) genes.

In some embodiments, analyzing the isolated immune cell comprises counting number or percentage of immune cells that bind to the bait composition and determining if the number or percentage of immune cells is above a threshold level, wherein the number or percentage of immune cells above a threshold level is indicative of cancer in the individual. In some embodiments, the immune cells are T cells (e.g., total T cells). In some embodiments, the immune cells are CD8+ T cells. In some embodiments, the immune cells are CD4+ T cells. In some embodiments, the immune cells are memory T cells. In some embodiments, the threshold is at least about 10, 20, 30, 40, 50, 75, 100, 150, 200, 250, 500, 1000, 2000, 3000, 4000, or 5000 immune cells that bind to the bait composition within a total of about 1×10⁵, 5×10⁵, 1×10⁶, 2×10⁶, 6×10⁶, 1×10⁷, 2×10⁷, 5×10⁷, or 1×10⁸ cells (e.g., immune cells) obtained in the sample (e.g., blood) from the individual. In some embodiments, at least about 1×10⁵ CD8 cells are analyzed, and cut-offs are set to more than 0.001% (such as more than about 0.001%, 0.002%, 0.003%, 0.004%, or 0.005% of CD8 cells and more than at least 2, 3, 4, 5, 6, 7, 8, 9, or 10 events. In some embodiments, at least about 1×10⁵ CD8 cells are analyzed, and cut-offs are set to more than 0.005% of CD8 cells and more than at least about 10 events.

Analysis of the immune repertoire usually involves one or more diversity indexes, such as Shannon entropy, clonality, and high-expanded clone (HEC) ratio, which are used to evaluate the amplification status of different TCR sequences and determine whether there is a high expansion of a few T-cell clones. See e.g., Li et al., Cancer Commun (Lond). 2020 October; 40(10): 473-483. It has been found that the highest diversity was found in peripheral blood samples of the healthy population according to the calculation results of Shannon entropy. Moreover, TCR repertoire diversity in sentinel lymph nodes from patients with tumor was higher than in tumor tissues.

In some embodiments, analyzing immune cells comprises analyzing repertoire diversity (e.g., Shannon diversity) of T cells obtained from the individual. In some embodiments, analyzing repertoire diversity comprises comparing the repertoire diversity of T cells obtained from the individual to that of T cells from a reference individual (such as a healthy individual, or a group of healthy individuals) and determine if the repertoire diversity is below a threshold, wherein the repertoire diversity (e.g., Shannon diversity) being below a threshold diversity is indicative of cancer in the individual. In some embodiments, the threshold is a Shannon diversity that is lower (e.g., at least about 5%, 10%, 15%, 20%, 25% or 30%) lower than that of T cells from a reference individual, it is indicative of cancer in the individual.

In some embodiments, analyzing the isolated immune cell comprises generating a signature profile. In some embodiments, the signature profile is associated with a specific cancer. In some embodiments, the cancer is hepatocellular carcinoma (HCC), and the signature profile comprises a three gene signature, wherein the three genes are CXCR2, CCR2 and EP400. See e.g., Shi et al., Eur J Cancer. 2014 March; 50(5):928-36.

In some embodiments, analyzing the isolated immune cell comprises generating a CD8 and/or CD4 T cell signature profile (e.g., gene expression profile (RNA-seq), gene rearrangement, 5mC, 5hmC or 5caC profile).

In some embodiments, analyzing the isolated immune cell comprises sequencing one or more nucleic acids and generating a library. In some embodiments, the one or more nucleic acids are repertoire related nucleic acids (e.g., VDJ gene-related DNA or RNA). In some embodiments, analyzing the isolating immune cells further comprises analyzing repertoire of the T cells as described above.

In some embodiments, analyzing the sequences of the one or more nucleic acids comprises a) obtaining an enriched sample from the isolated immune cell, wherein the enriched sample is enriched for the one or more nucleic acids and b) sequencing the one or more nucleic acids in the enriched sample.

In some embodiments, analyzing the isolated immune cell further comprises subjecting the isolated immune cell to mass spectrometry analysis. In some embodiments, analyzing the isolated immune cell further comprises subjecting the isolated immune cell to Assay for Transposase-Accessible Chromatin using sequencing (ATAC)-sequencing. In some embodiments, analyzing the isolated immune cell further comprises subjecting the isolated immune cell to chromatin immunoprecipitation (ChIP)-sequencing.

Epigenetic modifications of tumor specific immune cells (e.g., T cells, e.g., CD8+ T cells) have been reported. See e.g., Yang et al., Genome Biol 21, 2 (2020); Villanueva et al., Trends Immunol. 2020 August; 41 (8): 676-691. In some embodiments, analyzing the isolated immune cell further comprises identifying one or more epigenetic modifications in the T cell receptor genes of the isolated immune cell. In some embodiments, the one or more epigenetic modifications comprises histone acetylation, histone ubiquitination, and/or histone methylation. In some embodiments, the one or more epigenetic modifications comprises DNA or RNA methylation, hydroxylation and/or histone glycosylation.

### Individual

In some embodiments, the individual described herein is a mammal (such as a human, dog, cat, or horse). In some embodiments, the individual is a human.

In some embodiments, the individual has not previously been diagnosed as having a cancer (e.g. any cancer or a specific type of cancer).

In some embodiments, the individual has previously been diagnosed as having a cancer.

In some embodiments, the individual has minimal residual disease (MRD). In some embodiments, the individual has minimal residual cancer. In some embodiments, the minimal residual cancer is seen after the cancer was surgical resected or cured. In some embodiments, the minimal residual disease is too minimal to be detected by imaging instruments (e.g., a routinely used or standard imaging instrument used for detection of the cancer). In some embodiments, the location of the minimal residual disease is diverse. In some embodiments, the minimal residual cancer is a result of immune escape or resistance to treatment. In some embodiments, the individual has been previously treated for cancer and exhibits no pathological symptom of a cancer after the treatment.

In some embodiments, the individual is at risk of developing cancer. In some embodiments, the risk of having cancer is based on any one or more factors selected from the group consisting of family history, mutations, environmental factors, and age. In some embodiments, the individual is predicted by a doctor to have a risk of at least 20%, 30%, 40%, or 50% to develop a cancer (such as any specific kind of cancer) based upon any one or more factors selected from the group consisting of family history, mutations, environmental factors, and age.

In some embodiments, the individual is a human and is at least about 50 years old (e.g., such as at least about 50, 60, 70, or 80 years old).

In some embodiments, the individual is no more than 14 years old.

In some embodiments, the individual is a male. In some embodiments, the individual is a female.

### Cancer

The cancer described herein can be a cancer in general, or any type of cancer. In some embodiments, the cancer is a solid tumor. In some embodiments, the cancer is a liquid cancer.

In some embodiments, the cancer is a carcinoma, a sarcoma, a myeloma, a leukemia, a lymphoma, a blastoma, a germ cell tumor, or any combination thereof. In some embodiments, the cancer is a squamous cell carcinoma or an adenocarcinoma.

In some embodiments, the cancer is selected from the group consisting of: small cell lung cancer, non-small-cell lung cancer, nasopharyngeal cancer, colorectal cancer, anal cancer, liver cancer, bladder cancer, testicular cancer, cervical cancer, ovarian cancer, gastric cancer, esophageal cancer, head-and-neck cancer, pancreatic cancer, prostate cancer, renal cancer, thyroid cancer, melanoma cancer, and breast cancer.

In some embodiments, the cancer is a recurrent cancer.

### Generating Report

In some embodiments, the method of analyzing a sample of an individual further comprises generating a report comprising information about the cancer status in the individual.

In some embodiments, the information about cancer status comprises likelihood of the presence of a cancer, classification of cancer, type of cancer, nature of cancer, origin of cancer, stage of cancer, likelihood of cancer progression, likelihood of developing one or more cancer symptoms, and/or treatment options for the individual.

The information about the cancer status is based upon the results gathered from analyzing the immune cells that bind to the bait composition as discussed above. In some embodiments, the likelihood of the presence of a cancer is wholly or at least partly based upon the number and/or percentage of immune cells (e.g., T cells) that bind to the bait composition. For example, when the number and/or percentage of immune cells (e.g., T cells, e.g., CD8 T cells, e.g., CD4 T cells) that bind to the bait composition is significantly above a threshold level (such as a threshold level described above), the generated report comprises the information that the cancer is present in the individual. When the number and/or percentage of immune cells (e.g., T cells, e.g., CD8 T cells, e.g., CD4 T cells) is near or above threshold but not significantly above threshold, the generated report comprises the information that there is a median or high likelihood of the presence of the cancer.

In some embodiments, the likelihood of the presence of a cancer is wholly or at least partly based upon the repertoire diversity of T cells that bind to the bait composition. For example, when the repertoire diversity (e.g., Shannon diversity) of the T cells (e.g., CD8 T cells, e.g., CD4 T cells) that bind to the bait composition is significantly below the threshold, the generated report comprises the information that the cancer is present in the individual. When the repertoire diversity (e.g., Shannon diversity) of the T cells (e.g., CD8 T cells, e.g., CD4 T cells) that bind to the bait composition is near or below the threshold but not significantly below the threshold, the generated report comprises the information that there is a median or high likelihood of the presence of the cancer.

In some embodiments, the information about cancer status comprises gene expression profile signature, a gene mutation profile signature, and/or an epigenetic modification signature. In some embodiments, the signature epigenetic modification comprises a DNA methylation signature and a histone glycosylation signature.

### EXAMPLES

The examples below are intended to be purely exemplary of the invention and should therefore not be considered to limit the invention in any way. The following examples and detailed description are offered by way of illustration and not by way of limitation.

### Example 1

### A. Methods

### HLA Purification

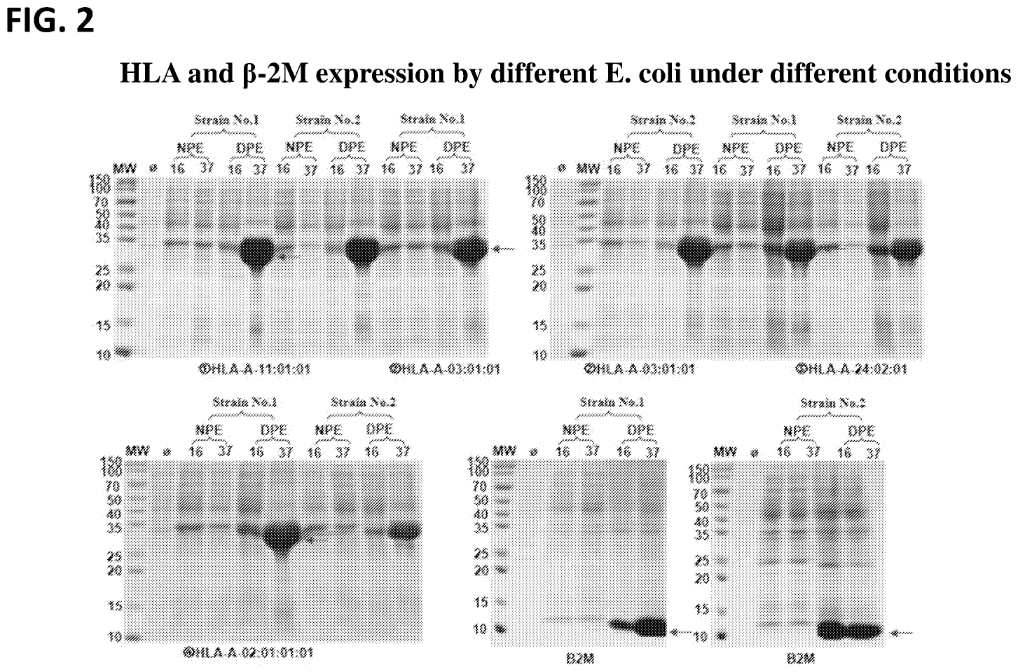

HLA-A:02:01, HLA-A:24:02, HLA-A:11:01, HLA-A:03:01, and β-2M were expressed in Escherichia coli. The purified subunits were confirmed by Coomassie blue staining. See e.g., FIG. 2.

### Kras Mutation Specific Neoantigen Library

Neoantigens comprising one of the four most common Kras mutations, including Kras G12V, Kras G12D, Kras G12R, Kras G12C were selected for building the exemplified neoantigen library. Twenty predicated neoantigens for each mutation were designed according to the features of neoantigens. These features include binding affinity with MHC molecules and/or T cells, binding stability, mutation sequence, and public neoantigens library. See e.g., FIG. 1.

### Neoantigen Synthesis

The neoantigen library was synthesized by Nanjing Peptide Biotech Ltd. The purity of peptide was above 95%.

Tetramers for specific neoantigens were assembly in vitro. The brief procedure was as following.

1. Put a 100 ml beaker with sterilized magnetic pole into a 4° C. refrigerator for precooling. Add reduced glutathione to make its final concentration 5 mM; Add oxidized glutathione, and the final concentration is 0.5 mM; The storage concentration is 100 mM (prepared with isopropanol) and the final concentration is 0.2 mM in the −20° C. refrigerator.

2. Take 100 nmol MHC heavy chain and 200 nmol MHC light chain, respectively, and thaw them (previously dissolved with 6M guanidine hydrochloride). Add 5 mL injection buffer (see solution configuration), and use a pipette to mix it evenly to avoid precipitation caused by high local protein concentration.

3. Use 100 ul DMSO to fully dissolve the mixture of the peptides (e.g., mixture of neoantigens comprising G12V, G12D, G12R, or G12C) with purity ≥95%. Depending on the solubility of the peptides, 30 mg of the peptide was dissolved in a minimum volume (e.g., 1 mL) of DMSO.

4. Put the folding system (i.e., the system for assembling MHC-peptide tetramer mixture) into a magnetic stirrer at 4° C. and stir at high speed. Use a 1 mL pipette to slowly drop the peptides into the stirring folding system. Avoid excessive local peptide concentration and add it drop by drop.

5. Put the prepared MHC heavy chains and MHC light chains on the ice to be precooled for use. After the preparation is completed, use a 1 mL sterile pipette to add 5 ml light chain and heavy chain suspension to the high-speed stirred reaction system. Avoid precipitation caused by excessive local protein concentration. Use the same method when adding the heavy chain into the folding system, and add the folding buffer as close to the mixing rod as possible.

6. Return the folding reaction system to 4° C. and incubate it for 12 hours (overnight). During this period, the magnetic stirrer was used to stir gently (the rotation speed was controlled at 150-200 rpm/min) to make the folding reaction more complete.

7. After incubation (i.e., assembly of MHC-peptide mixture), add 1 μM MHC heavy chain prepared as in step 2 per method described in step 5, and sufficiently incubate for 10 hours at 4° C.

8. After incubation (for example, before leaving at night), add an additional 1 μM heavy chain as described above (step 5) and return to 10° C. for at least overnight and at most 5 days.

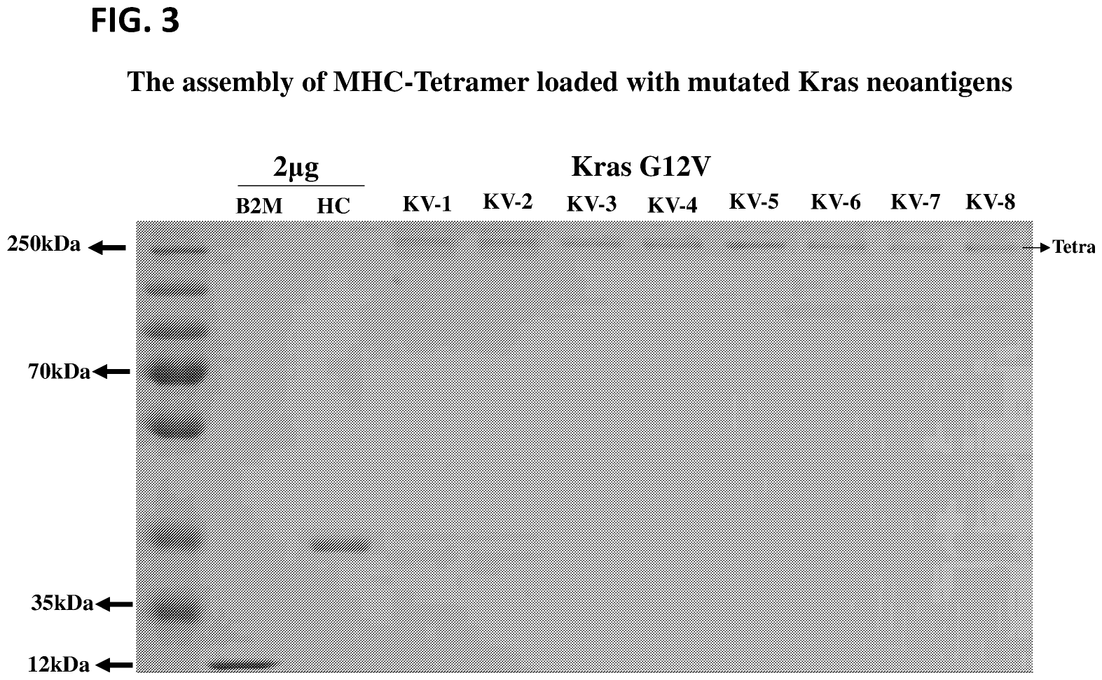

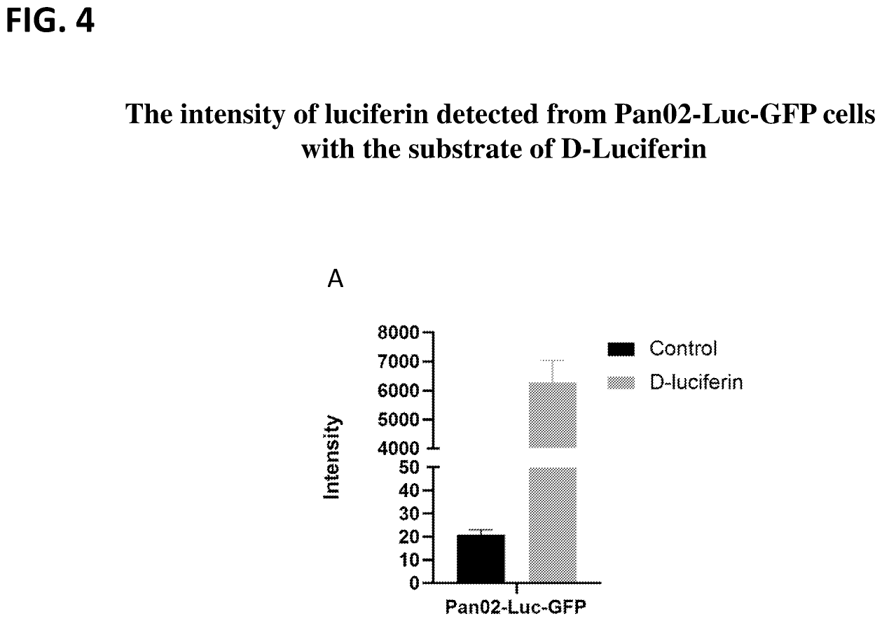

9. Purify the assembled tetramers by anion-exchange chromatography. See e.g., FIG. 3 for exemplified successfully assembled MHC tetramers with neoantigen peptides comprising G12V mutation. MHC tetramers with neoantigen peptides comprising G12R, G12D or G12C were also generated. Construction of pancreatic tumor cells line expressing multiple Kras mutation specific neoantigens.

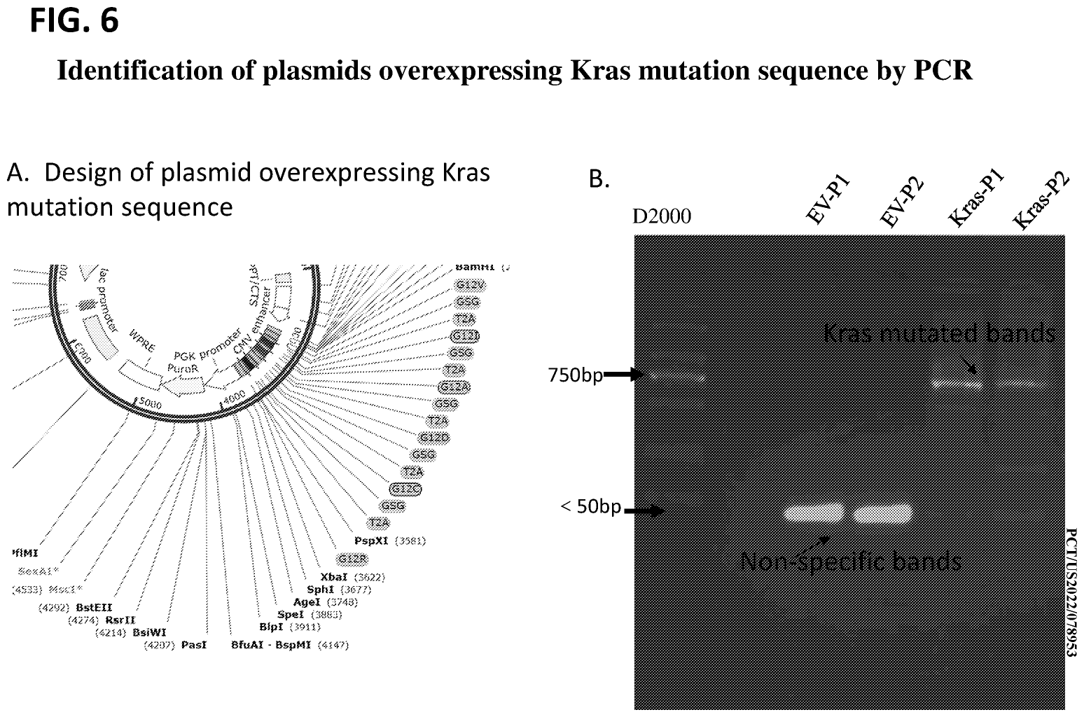

The five most common Kras mutation in pancreatic cancer were co-overexpressed in one plasmid and transfected into cells to stably expression in pancreatic tumor cells Pan02-Luc-GFP or 266-6-luc to produce excess Kras mutation specific neoantigens. The expression of transfected Kras mutant sequence were detected by PCR and mass spectrum. See e.g., FIG. 6.

### Animal Model of Tumor Cells Co-Overexpressing Five Most Common Kras Mutations

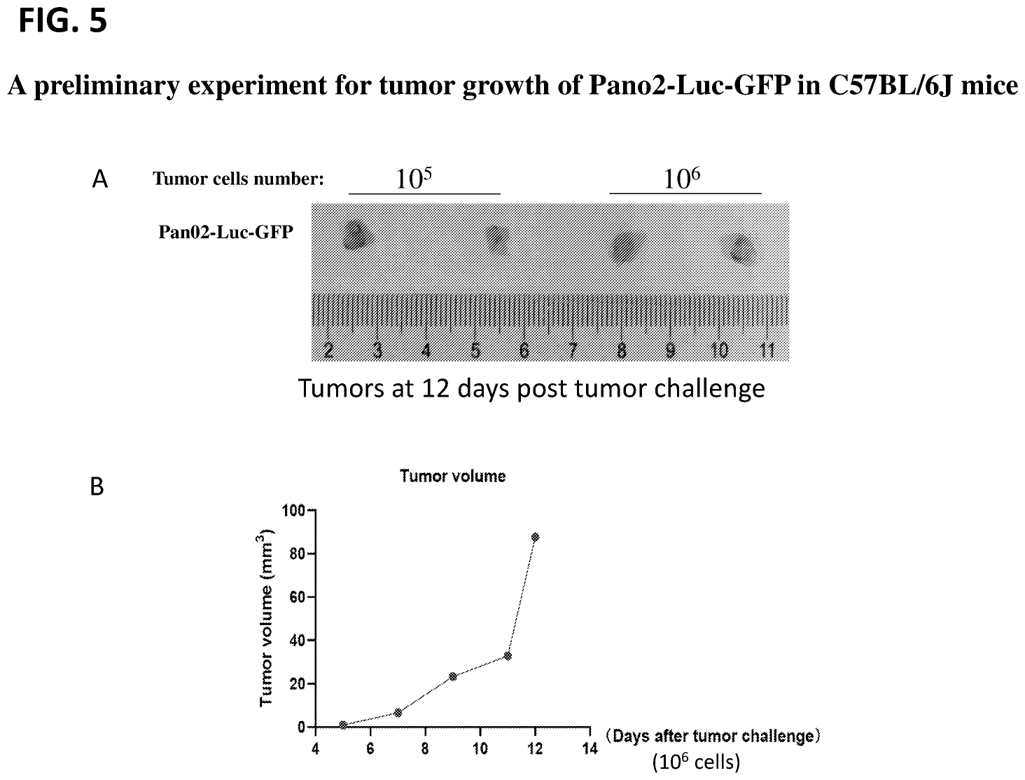

Subcutaneous tumor model: Tumor cells Pan02-Luc-GFP (e.g., 4×10⁵) which have different gradient antigen peptide combinations were inoculated subcutaneously in C57BL/6J mice. The existence of T cells targeting neoantigens comprising Kras mutations were evaluated by contacting peripheral blood cells with tetramers targeting specific antigen library prepared as above various days post inoculation respectively to determine the time point of early diagnosis on tumor size and immune stimulation time. The tumor size was recorded by bioluminescence. See FIG. 5.

Intravenous model: Tumor cells which have different gradient overexpression neoantigen peptide combinations were inoculated through the tail vein. The existence of T cells targeting neoantigens comprising Kras mutations were evaluated by incubating tetramers targeting specific neoantigen library prepared as above on day 0, day 1, day 4, day 8, day 16, and day 24 respectively, so as to determine the time point of early diagnosis on tumor size and immune stimulation time. The number of residual tumor cells in the peripheral system were monitored in real time through the autofluorescence of the inoculated cells.

The Collection of Peripheral Blood from Pancreatic Cancer Patients and T Cells Capture

The fresh blood was collected from pancreatic cancer patients and the red blood cells were lysed by ACK lysis buffer for 5 min at room temperature. Then, the white cell pellet was washed for two times before cell staining with cool PBS on ice. Fcblock antibody was used to pretreated samples for 10 min at 4° C., and the fluorescence conjugated primary antibodies targeting CD45, CD3e, or CD8 along with Tetramer were added for cell specific staining for 30 min in dark at 4° C. After staining, the cells were washed by cool PBS (containing 2% FBS) for two time. Finally, the cells were detected by flow cytometry immediately and the data was analyzed by Flow Jo V10 software.

### B. Results

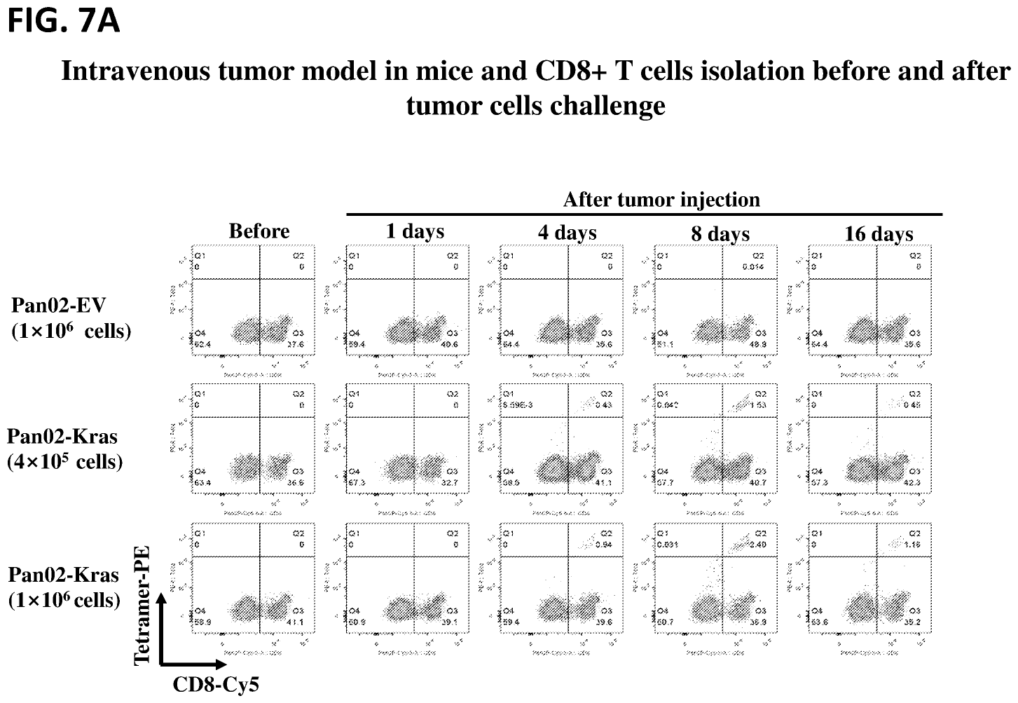

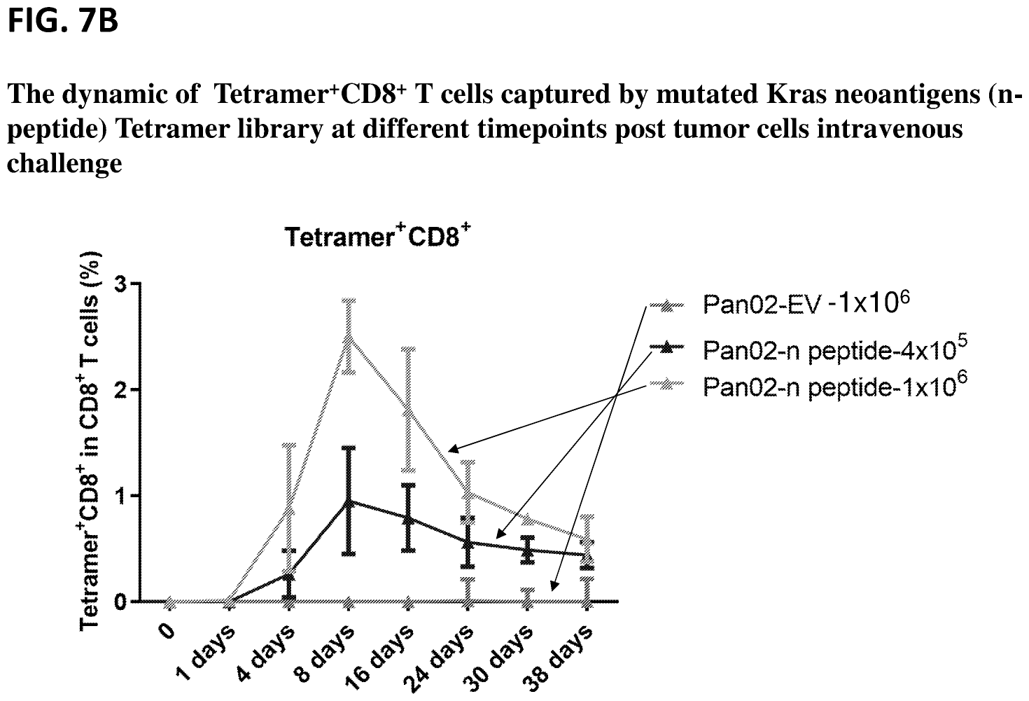

As shown in FIGS. 7A-7B, 8, 9A-9B, Kras mutation associated neoantigen-specific T cells were successfully detected in mice intravenously or subcutaneously inoculated with tumor cells (of as low as 10⁴ cells, see, e.g., FIG. 8) expressing Kras mutation associated neoantigens as early as day 4 after inoculation (see e.g., FIG. 9A-9B) which is prior to the any detection via bioluminescence (see e.g., FIG. 10A-10B), or in pancreatic cancer patients harboring Kras mutations (e.g., see FIGS. 11A-11C and 12). These results demonstrated successful applications of a tetramer based screening platform for early cancer screening.

SEQUENCE TABLE

| SEQ ID | | Nucleotide or Amino |
|---|---|---|
| NO. | Description | Acid Sequence |
| 1. | Exemplary NSCLC | VYCEEYYLF |
| | neoantigenic peptide | |
| 2. | Exemplary NSCLC | YQANVVWKV |
| | neoantigenic peptide | |
| 3. | Exemplary NSCLC | ALYFNSQWK |
| | neoantigenic peptide | |
| 4. | Exemplary NSCLC | KLLSFHSV |
| | neoantigenic peptide | |
| 5. | Exemplary NSCLC | YLNEAVFNFV |
| | neoantigenic peptide | |
| 6. | Exemplary NSCLC | FYMHEYPEGW |
| | neoantigenic peptide | |
| 7. | Exemplary NSCLC | EIDLPRELEY |
| | neoantigenic peptide | |
| 8. | Exemplary NSCLC | KQDGYDSV |
| | neoantigenic peptide | |
| 9. | Exemplary NSCLC | LLQHYLLYR |
| | neoantigenic peptide | |
| 10. | Exemplary NSCLC | TTARMRTMR |
| | neoantigenic peptide | |
| 11. | Exemplary NSCLC | HTFHLQDHH |
| | neoantigenic peptide | |
| 12. | Exemplary NSCLC | STTGATDLK |
| | neoantigenic peptide | |
| 13. | Exemplary NSCLC | MTFAETYPA |
| | neoantigenic peptide | |
| 14. | Exemplary NSCLC | NLLLIRGFK |
| | neoantigenic peptide | |
| 15. | Exemplary NSCLC | HGYFWFMGR |
| | neoantigenic peptide | |
| 16. | Exemplary NSCLC | FVLAALMEY |
| | neoantigenic peptide | |
| 17. | Exemplary NSCLC | RLRRLPVPR |
| | neoantigenic peptide | |
| 18. | Exemplary NSCLC | QSLVPAHPK |
| | neoantigenic peptide | |
| 19. | Exemplary NSCLC | RSFLSWDSR |
| | neoantigenic peptide | |
| 20. | Exemplary NSCLC | RIQGYIIEK |
| | neoantigenic peptide | |
| 21. | Exemplary NSCLC | ALNGMPLLK |
| | neoantigenic peptide | |
| 22. | Exemplary NSCLC | GTSESRETR |
| | neoantigenic peptide | |
| 23. | Exemplary NSCLC | IFSHPLYNI |
| | neoantigenic peptide | |
| 24. | Exemplary NSCLC | AAYLLFYQR |
| | neoantigenic peptide | |
| 25. | Exemplary NSCLC | SYVNILRAI |
| | neoantigenic peptide | |
| 26. | Exemplary NSCLC | RVEPVNYPK |
| | neoantigenic peptide | |
| 27. | Exemplary NSCLC | GYMGQQNEL |
| | neoantigenic peptide | |
| 28. | Exemplary NSCLC | RTQEARPPR |
| | neoantigenic peptide | |
| 29. | Exemplary NSCLC | RTASEDHPR |
| | neoantigenic peptide | |
| 30. | Exemplary NSCLC | VTIFVYDVK |
| | neoantigenic peptide | |
| 31. | Exemplary NSCLC | VVCIDAFLK |
| | neoantigenic peptide | |
| 32. | Exemplary NSCLC | STDQPVIPK |
| | neoantigenic peptide | |
| 33. | Exemplary NSCLC | SVAWPQDRR |
| | neoantigenic peptide | |
| 34. | Exemplary NSCLC | PTFGLSILK |
| | neoantigenic peptide | |
| 35. | Exemplary NSCLC | SYGLILLAF |
| | neoantigenic peptide | |
| 36. | Exemplary NSCLC | VFPLLFGTF |
| | neoantigenic peptide | |
| 37. | Exemplary NSCLC | YYLCLRHRL |
| | neoantigenic peptide | |
| 38. | Exemplary NSCLC | TSFPLDANK |
| | neoantigenic peptide | |
| 39. | Exemplary NSCLC | SLMVCNHDK |
| | neoantigenic peptide | |
| 40. | Exemplary NSCLC | SYIYILIII |
| | neoantigenic peptide | |
| 41. | Exemplary NSCLC | GVFRRCWEK |
| | neoantigenic peptide | |
| 42. | Exemplary NSCLC | RSAAIASEK |
| | neoantigenic peptide | |
| 43. | Exemplary NSCLC | SSTHPHFVR |
| | neoantigenic peptide | |
| 44. | Exemplary NSCLC | NVLEINFIK |
| | neoantigenic peptide | |
| 45. | Exemplary NSCLC | SMVPVMYQK |
| | neoantigenic peptide | |
| 46. | Exemplary NSCLC | SVYCIGQRR |
| | neoantigenic peptide | |
| 47. | Exemplary NSCLC | YQMLSFVHK |
| | neoantigenic peptide | |
| 48. | Exemplary NSCLC | LYSFISRFI |
| | neoantigenic peptide | |
| 49. | Exemplary NSCLC | FSFRSCNFK |
| | neoantigenic peptide | |
| 50. | Exemplary NSCLC | ISYAKYFPK |
| | neoantigenic peptide | |
| 51. | Exemplary NSCLC | AEYQDMHSY |
| | neoantigenic peptide | |
| 52. | Exemplary NSCLC | VEHINISQDW |
| | neoantigenic peptide | |
| 53. | Exemplary NSCLC | RGRMQTASL |
| | neoantigenic peptide | |
| 54. | Exemplary NSCLC | VRINTARPV |
| | neoantigenic peptide | |
| 55. | Exemplary NSCLC | RRSMLFARH |
| | neoantigenic peptide | |
| 56. | Exemplary NSCLC | KTDTGVHATL |
| | neoantigenic peptide | |
| 57-76 | See FIG. 1 or sequence | |
| | listing | |

1. A method of analyzing a sample of an individual exhibiting no pathological symptom of a cancer, the method comprising:
a) contacting the sample with a bait composition comprising a display moiety comprising a cancer neoantigenic peptide under a condition sufficient for an immune cell to bind to the display moiety;
b) isolating an immune cell associated with the display moiety; and
c) analyzing the isolated immune cell.

2. The method of claim 1, wherein the method further comprises culturing the isolated immune cell prior to the analyzing step.

3. The method of claim 1, wherein the display moiety comprises two or more neoantigenic peptides.

4. The method of claim 1, wherein the display moiety comprises four neoantigenic peptides.

5. The method of any one of claims 3-4, wherein the two or more neoantigenic peptides in the display moiety are the same.

6. The method of any one of claims 1-5, wherein the neoantigenic peptide has one or more of the following characteristics:
a) having a binding affinity of about 1 nM to about 5000 nM to an MHC molecule;
b) having a binding affinity of about 1 nM to about 5000 nM to a cognate TCR molecule;
c) having a mutation relative to a wildtype peptide, optionally at the third amino acid position counting from the N-terminus;
d) is hydrophobic; and
e) has high content of aromatic residues.

7. The method of claim 1-6, wherein the neoantigenic peptide has low immunogenicity.

8. The method of any one of claims 1-7, wherein the display moiety comprises an MHC molecule complexed with the neoantigenic peptide.

9. The method of claim 8, wherein the MHC molecule is a MHC class I molecule.

10. The method of claim 9, wherein the MHC class I molecule is selected from the group consisting of HLA-A, HLA-B, and HLA-C.

11. The method of claim 9 or 10, wherein the peptide is about 8 to about 10 amino acids long.

12. The method of claim 8, wherein the MHC is a MHC class II molecule.

13. The method of claim 12, wherein the MHC class II molecule is selected from the group consisting of HLA-DQ and HLA-DR.

14. The method of claim 12 or 13, wherein the neoantigenic peptide is about 10 to about 20 amino acids long.

15. The method of any one of claims 8-14, wherein the display moiety comprises two or more different kinds of MHC class I molecules selected from the group consisting of HLA-A*24:02, HLA-A*11:01, HLA-A*02:01, and HLA-A*03:01.

16. The method of any one of claims 1-15, wherein the display moiety comprises a particle.

17. The method of claim 16, wherein the particle is selected from the group consisting of: a surface, a nanoparticle, a bead, and a polymer.

18. The method of claim 16 or 17, wherein the particle is a dextran particle.

19. The method of claim 16 or 17, wherein the particle is a magnetic nanoparticle or polystyrene nanoparticle.

20. The method of claim 16 or 17, wherein the particle is an agarose bead or a sepharose bead.

21. The method of any one of claims 16-20, wherein the neoantigenic peptide or MHC is directly attached to the particle.

22. The method of any one of claims 16-20, wherein the neoantigenic peptide or MHC is attached to the particle via a binding pair comprising a first binding component attached to the neoantigenic peptide and a second binding component bound to the particle.

23. The method of any one of claims 1-15, wherein the display moiety comprises a cell.

24. The method of claim 23, wherein the cell comprises a polynucleotide encoding the neoantigenic peptide.

25. The method of claim 24, wherein the polynucleotide encodes a plurality of neoantigenic peptides.

26. The method of any one of claims 1-25, wherein the display moiety further comprises a detectable label.

27. The method of claim 26, wherein the detectable label is a fluorophore.

28. The method of claim 27, wherein the isolating step comprises using fluorescence-activated cell sorting (FACS).

29. The method of any one of claims 1-28, wherein the isolating step comprises separating immune cells associated with the display moiety from the rest of the sample.

30. The method of any one of claims 1-29, wherein the isolated immune cell is selected from the group consisting of: a cytotoxic T cell, a memory T cell, and a tumor infiltrating T cell.

31. The method of any one of claims 1-29, wherein the isolated immune cell is a B cell.

32. The method of any one of claims 1-31, wherein the isolated immune cell is an isolated single immune cell.

33. The method of any one of claims 1-31, wherein the isolated immune cell is in a mixture of immune cells.

34. The method of claim 33, wherein the mixture of immune cells is a mixture comprising T cells, memory T cells, macrophage cells, or dendritic cells, or combinations thereof.

35. The method of any one of claims 1-34, wherein analyzing the isolated immune cell comprises detecting the isolated immune cell.

36. The method of any one of claims 1-35, wherein analyzing the isolated immune cell comprises quantifying the isolated immune cell.

37. The method of any one of claims 1-36, wherein analyzing the isolated immune cell comprises sequencing one or more nucleic acids in the isolated immune cell.

38. The method of claim 37, wherein analyzing the isolated immune cell further comprises analyzing the sequences of the one or more nucleic acids.

39. The method of claim 37 or claim 38, wherein the one or more nucleic acids is a TCR sequence.

40. The method of any one of claims 37-39, wherein analyzing the sequences of the one or more nucleic acids comprises whole genome sequencing.

41. The method of any one of claims 37-40, wherein analyzing the sequences of the one or more nucleic acids comprises RNAseq sequencing.

42. The method of any one of claims 37-39, wherein analyzing the sequences of the one or more nucleic acids comprises:
a) obtaining an enriched sample from the isolated immune cell, wherein the enriched sample is enriched for the one or more nucleic acids; and
b) sequencing the one or more nucleic acids in the enriched sample.

43. The method of any one of claims 1-42, wherein analyzing the isolated immune cell further comprises subjecting the isolated immune cell to mass spectrometry analysis.

44. The method of any one of claims 1-43, wherein analyzing the isolated immune cell further comprises identifying one or more epigenetic modifications in the isolated immune cell.

45. The method of claim 44, wherein the more or more epigenetic modifications comprises DNA methylation and/or histone glycosylation.

46. The method of any one of claims 1-45, wherein the individual has not previously been diagnosed as having a cancer.

47. The method of claim 46, wherein the individual is at risk of having cancer.

48. The method of any one of claims 1-45, wherein the individual has been previously treated for cancer and exhibits no pathological symptom of a cancer after the treatment.

49. The method of any one of claims 1-48, wherein the individual is a human.

50. the method of claim 49, wherein the individual is at least about 50 years old.

51. The method of any one of claims 1-50, wherein the sample is selected from the group consisting of: blood, plasma, and a peripheral blood mononuclear cell (PMBC) sample.

52. The method of any one of claims 1-51, further comprising generating a report comprising information about the cancer status in the individual.

53. The method of claim 52, wherein the information about cancer status comprises: classification of cancer; type of cancer; nature of cancer; origin of cancer; stage of cancer; likelihood of cancer progression; likelihood of developing one or more cancer symptoms; molecular diagnosis; NGS pathology; and/or treatment options for the individual.

54. The method of any one of claims 1-53, wherein the bait composition comprises a plurality of different display moieties.

55. The method of claim 54, wherein the each of the plurality of different display moieties in the bait composition comprises a different neoantigenic peptide.

56. The method of claim 54 or 55, wherein the plurality of different display moieties in the bait composition comprises at least two different display moieties, each comprising a different MHC molecule.

57. The method of claim 56, wherein the plurality of different display moieties in the bait composition comprises at least four different display moieties, each comprising a different MHC molecule.

58. The method of claim 56, wherein the plurality of different display moieties in the bait composition comprises at least 100 different display moieties, each comprising a different MHC molecule.

59. The method of any one of claims 56-58, wherein each of the different display moieties comprising different MHC molecules comprises a different detectable label.

60. The method of claim 59, wherein the detectable label is a fluorophore.

61. The method of claim 60, wherein the isolating step comprises using fluorescence-activated cell sorting (FACS).

62. The method of any one of claims 59-61, wherein the isolating step comprises separating immune cells associated with each of the different display moieties comprising different MHC molecules into different populations.

63. The method of any one of claims 1-62, wherein the method comprises contacting each of a plurality of different display moieties with a sample from the individual separately and isolating the immune cell associated with each of the different display moiety.

64. The method of any one of claims 48-63, wherein the method comprises:
a) analyzing a pre-treatment sample from the individual prior to anti-cancer therapy and a post-treatment sample from the individual according to any one of the methods of claims 48-56; and
b) identifying a difference in characteristics of the isolated immune cell from the pre-treatment sample and the isolated immune cell from the post-treatment sample.

65. A method of detecting cancer in an individual, comprising: analyzing a sample from the individual according to any one of claims 1-64,
wherein a predetermined characteristic of the isolated immune cell is indicative of cancer in the individual.

66. The method of claim 65, wherein the predetermined characteristic of the isolated immune cell comprises the presence of the isolated immune cell.

67. The method of claim 66, wherein the predetermined characteristic of the isolated immune cell comprises a quantity of the isolated immune cell above a threshold level.

68. The method of any one of claims 65-67, wherein the predetermined characteristic of the isolated immune cell comprises a gene expression profile signature, a gene mutation profile signature, and/or an epigenetic modification signature.

69. The method of claim 68, wherein the signature epigenetic modification comprises a DNA or RNA methylation, hydroxylation signature and a histone acetylation, methylation and/or glycosylation signature.

70. A method of detecting residual cancer in an individual, wherein the individual has been previously treated with an anti-cancer therapy and exhibits no pathological symptom of cancer after treatment, the method comprising analyzing a post-treatment sample from the individual according to the method of any one of claims 48-56,
wherein a predetermined characteristic of the isolated immune cell from the post-treatment sample is indicative of residual cancer in the individual.

71. The method of claim 70, wherein the method comprises:
a) analyzing a pre-treatment sample from the individual prior to anti-cancer therapy and a post-treatment sample from the individual according to the method of any one of claims 48-56, and
b) comparing the characteristics of the isolated immune cells from the pre-treatment sample and isolated immune cells from the post-treatment sample;
wherein a predetermined difference in characteristics of the isolated immune cell from the pre-treatment sample and the isolated immune cell from the post-treatment sample is indicative of residual cancer in the individual.

72. A method of treating a cancer in an individual, comprising
a) diagnosing the individual as having cancer according to the method of any one of claims 65-71; and
b) subjecting the individual to an anti-cancer therapy.

73. The method of claim 72, wherein the anti-cancer therapy is not an immunotherapy.

74. The method of any one of claims 1-73, wherein the cancer is a solid tumor.

75. The method of any one of claims 1-74, wherein the cancer is a carcinoma, a sarcoma, a myeloma, a leukemia, a lymphoma, a blastoma, a germ cell tumor, or any combination thereof.

76. The method of any one of claims 1-75, wherein the cancer is a squamous cell carcinoma or an adenocarcinoma.

77. The method according to any one of claims 1-76, wherein the cancer is selected from the group consisting of: small cell lung cancer, non-small-cell lung cancer, nasopharyngeal cancer, colorectal cancer, anal cancer, liver cancer, bladder cancer, testicular cancer, cervical cancer, ovarian cancer, gastric cancer, esophageal cancer, head-and-neck cancer, pancreatic cancer, prostate cancer, renal cancer, thyroid cancer, melanoma cancer, and breast cancer.


SEQUENCE LISTING (76 sequences)

SEQ ID NO: 1 - protein, 9 aa, synthetic construct
VYCEEYYLF

SEQ ID NO: 2 - protein, 9 aa, synthetic construct
YQANVVWKV

SEQ ID NO: 3 - protein, 9 aa, synthetic construct
ALYFNSQWK

SEQ ID NO: 4 - protein, 8 aa, synthetic construct
KLLSFHSV

SEQ ID NO: 5 - protein, 10 aa, synthetic construct
YLNEAVFNFV

SEQ ID NO: 6 - protein, 10 aa, synthetic construct
FYMHEYPEGW

SEQ ID NO: 7 - protein, 10 aa, synthetic construct
EIDLPRELEY

SEQ ID NO: 8 - protein, 8 aa, synthetic construct
KQDGYDSV

SEQ ID NO: 9 - protein, 9 aa, synthetic construct
LLQHYLLYR

SEQ ID NO: 10 - protein, 9 aa, synthetic construct
TTARMRTMR

SEQ ID NO: 11 - protein, 9 aa, synthetic construct
HTFHLQDHH

SEQ ID NO: 12 - protein, 9 aa, synthetic construct
STTGATDLK

SEQ ID NO: 13 - protein, 9 aa, synthetic construct
MTFAETYPA

SEQ ID NO: 14 - protein, 9 aa, synthetic construct
NLLLIRGFK

SEQ ID NO: 15 - protein, 9 aa, synthetic construct
HGYFWFMGR

SEQ ID NO: 16 - protein, 9 aa, synthetic construct
FVLAALMEY

SEQ ID NO: 17 - protein, 9 aa, synthetic construct
RLRRLPVPR

SEQ ID NO: 18 - protein, 9 aa, synthetic construct
QSLVPAHPK

SEQ ID NO: 19 - protein, 9 aa, synthetic construct
RSFLSWDSR

SEQ ID NO: 20 - protein, 9 aa, synthetic construct
RIQGYIIEK

SEQ ID NO: 21 - protein, 9 aa, synthetic construct
ALNGMPLLK

SEQ ID NO: 22 - protein, 9 aa, synthetic construct
GTSESRETR

SEQ ID NO: 23 - protein, 9 aa, synthetic construct
IFSHPLYNI

SEQ ID NO: 24 - protein, 9 aa, synthetic construct
AAYLLFYQR

SEQ ID NO: 25 - protein, 9 aa, synthetic construct
SYVNILRAI

SEQ ID NO: 26 - protein, 9 aa, synthetic construct
RVEPVNYPK

SEQ ID NO: 27 - protein, 9 aa, synthetic construct
GYMGQQNEL

SEQ ID NO: 28 - protein, 9 aa, synthetic construct
RTQEARPPR

SEQ ID NO: 29 - protein, 9 aa, synthetic construct
RTASEDHPR

SEQ ID NO: 30 - protein, 9 aa, synthetic construct
VTIFVYDVK

SEQ ID NO: 31 - protein, 9 aa, synthetic construct
VVCIDAFLK

SEQ ID NO: 32 - protein, 9 aa, synthetic construct
STDQPVIPK

SEQ ID NO: 33 - protein, 9 aa, synthetic construct
SVAWPQDRR

SEQ ID NO: 34 - protein, 9 aa, synthetic construct
PTFGLSILK

SEQ ID NO: 35 - protein, 9 aa, synthetic construct
SYGLILLAF

SEQ ID NO: 36 - protein, 9 aa, synthetic construct
VFPLLFGTF

SEQ ID NO: 37 - protein, 9 aa, synthetic construct
YYLCLRHRL

SEQ ID NO: 38 - protein, 9 aa, synthetic construct
TSFPLDANK

SEQ ID NO: 39 - protein, 9 aa, synthetic construct
SLMVCNHDK

SEQ ID NO: 40 - protein, 9 aa, synthetic construct
SYIYILIII

SEQ ID NO: 41 - protein, 9 aa, synthetic construct
GVFRRCWEK

SEQ ID NO: 42 - protein, 9 aa, synthetic construct
RSAAIASEK

SEQ ID NO: 43 - protein, 9 aa, synthetic construct
SSTHPHFVR

SEQ ID NO: 44 - protein, 9 aa, synthetic construct
NVLEINFIK

SEQ ID NO: 45 - protein, 9 aa, synthetic construct
SMVPVMYQK

SEQ ID NO: 46 - protein, 9 aa, synthetic construct
SVYCIGQRR

SEQ ID NO: 47 - protein, 9 aa, synthetic construct
YQMLSFVHK

SEQ ID NO: 48 - protein, 9 aa, synthetic construct
LYSFISRFI

SEQ ID NO: 49 - protein, 9 aa, synthetic construct
FSFRSCNFK

SEQ ID NO: 50 - protein, 9 aa, synthetic construct
ISYAKYFPK

SEQ ID NO: 51 - protein, 9 aa, synthetic construct
AEYQDMHSY

SEQ ID NO: 52 - protein, 10 aa, synthetic construct
VEHINISQDW

SEQ ID NO: 53 - protein, 9 aa, synthetic construct
RGRMQTASL

SEQ ID NO: 54 - protein, 9 aa, synthetic construct
VRINTARPV

SEQ ID NO: 55 - protein, 9 aa, synthetic construct
RRSMLFARH

SEQ ID NO: 56 - protein, 10 aa, synthetic construct
KTDTGVHATL

SEQ ID NO: 57 - protein, 8 aa, synthetic construct
  VARIANT 8: note = X = V, D, R, C, I, or A
KLVVVGAX

SEQ ID NO: 58 - protein, 9 aa, synthetic construct
  VARIANT 8: note = X = V, D, R, C, I, or A
KLVVVGAXG

SEQ ID NO: 59 - protein, 10 aa, synthetic construct
  VARIANT 8: note = X = V, D, R, C, I, or A
KLVVVGAXGV

SEQ ID NO: 60 - protein, 11 aa, synthetic construct
  VARIANT 8: note = X = V, D, R, C, I, or A
KLVVVGAXGVG

SEQ ID NO: 61 - protein, 8 aa, synthetic construct
  VARIANT 7: note = X = V, D, R, C, I, or A
LVVVGAXG

SEQ ID NO: 62 - protein, 9 aa, synthetic construct
  VARIANT 7: note = X = V, D, R, C, I, or A
LVVVGAXGV

SEQ ID NO: 63 - protein, 10 aa, synthetic construct
  VARIANT 7: note = X = V, D, R, C, I, or A
LVVVGAXGVG

SEQ ID NO: 64 - protein, 11 aa, synthetic construct
  VARIANT 7: note = X = V, D, R, C, I, or A
LVVVGAXGVGK

SEQ ID NO: 65 - protein, 8 aa, synthetic construct
  VARIANT 6: note = X = V, D, R, C, I, or A
VVVGAXGV

SEQ ID NO: 66 - protein, 9 aa, synthetic construct
  VARIANT 6: note = X = V, D, R, C, I, or A
VVVGAXGVG

SEQ ID NO: 67 - protein, 10 aa, synthetic construct
  VARIANT 6: note = X = V, D, R, C, I, or A
VVVGAXGVGK

SEQ ID NO: 68 - protein, 11 aa, synthetic construct
  VARIANT 6: note = X = V, D, R, C, I, or A
VVVGAXGVGKS

SEQ ID NO: 69 - protein, 8 aa, synthetic construct
  VARIANT 5: note = X = V, D, R, C, I, or A
VVGAXGVG

SEQ ID NO: 70 - protein, 9 aa, synthetic construct
  VARIANT 5: note = X = V, D, R, C, I, or A
VVGAXGVGK

SEQ ID NO: 71 - protein, 10 aa, synthetic construct
  VARIANT 5: note = X = V, D, R, C, I, or A
VVGAXGVGKS

SEQ ID NO: 72 - protein, 11 aa, synthetic construct
  VARIANT 5: note = X = V, D, R, C, I, or A
VVGAXGVGKSA

SEQ ID NO: 73 - protein, 8 aa, synthetic construct
  VARIANT 4: note = X = V, D, R, C, I, or A
VGAXGVGK

SEQ ID NO: 74 - protein, 9 aa, synthetic construct
  VARIANT 4: note = X = V, D, R, C, I, or A
VGAXGVGKS

SEQ ID NO: 75 - protein, 10 aa, synthetic construct
  VARIANT 4: note = X = V, D, R, C, I, or A
VGAXGVGKSA

SEQ ID NO: 76 - protein, 11 aa, synthetic construct
  VARIANT 4: note = X = V, D, R, C, I, or A
VGAXGVGKSAL